SEQUENCE LISTING (196 sequences)

SEQ ID NO: 1 - protein, 17 aa, synthetic construct
KSSQSLLNSGNQKNYLT

SEQ ID NO: 2 - protein, 7 aa, synthetic construct
WAATRES

SEQ ID NO: 3 - protein, 9 aa, synthetic construct
QNDYTYPLT

SEQ ID NO: 4 - protein, 5 aa, synthetic construct
NYWIE

SEQ ID NO: 5 - protein, 17 aa, synthetic construct
EIFPGSGTTKYNEKFKG

SEQ ID NO: 6 - protein, 12 aa, synthetic construct
GRFISTTRGFAY

SEQ ID NO: 7 - protein, 113 aa, synthetic construct
DIVMTQSPSSLTVTPGEKVTMSCKSSQSLLNSGNQKNYLTWYQQKPGQPPKLLIYWAATRESGVPDRFTGSGSGTDFTLTISSVQAEDLAVYYCQNDYTYPLTFGAGTKLELK

SEQ ID NO: 8 - protein, 121 aa, synthetic construct
QVQLQQSGAELTKPGASVKISCKATGYTFSNYWIEWVIQRPGHGLEWIGEIFPGSGTTKYNEKFKGKATFTADTSSNTAYMQLNSLTSEDSAVYYCARGRFISTTRGFAYWGQGTLVTVSA

SEQ ID NO: 9 - protein, 11 aa, synthetic construct
KASQDVSTAVA

SEQ ID NO: 10 - protein, 7 aa, synthetic construct
SASYRYT

SEQ ID NO: 11 - protein, 9 aa, synthetic construct
QQHFNTPRT

SEQ ID NO: 12 - protein, 5 aa, synthetic construct
SYAMS

SEQ ID NO: 13 - protein, 17 aa, synthetic construct
SINSGGFTYYPDSVKGR

SEQ ID NO: 14 - protein, 8 aa, synthetic construct
GGTTVFDY

SEQ ID NO: 15 - protein, 107 aa, synthetic construct
DIVMTQSHKFMSTSVGDRVSITCKASQDVSTAVAWYQQKPGHSPKLLIYSASYRYTGVPDRFTGSGSGADFTFTISSVQAEDLAVYFCQQHFNTPRTFGGGTKLEIK

SEQ ID NO: 16 - protein, 116 aa, synthetic construct
EVKLVESGGGLVKPGGSLKLSCAASGFTFSSYAMSWVRQTPEKRLEWVASINSGGFTYYPDSVKGRFTISRDNTRNILYLQLSSLRSEDTAMYYCARGGTTVFDYWGQGTTLTVSS

SEQ ID NO: 17 - protein, 11 aa, synthetic construct
KASQDVSTAVA

SEQ ID NO: 18 - protein, 7 aa, synthetic construct
SASYRYT

SEQ ID NO: 19 - protein, 9 aa, synthetic construct
QQHFNTPRT

SEQ ID NO: 20 - protein, 5 aa, synthetic construct
SYAMS

SEQ ID NO: 21 - protein, 16 aa, unidentified
SINSGGFTYYPDSVKG

SEQ ID NO: 22 - protein, 8 aa, synthetic construct
GGTTVFDY

SEQ ID NO: 23 - protein, 107 aa, synthetic construct
DIQMTQSPSSLSASVGDRVTITCKASQDVSTAVAWYQQKPGKAPKLLIYSASYRYTGVPSRFSGSGSGADFTFTISSLQPEDIATYYCQQHFNTPRTFGGGTKVEIK

SEQ ID NO: 24 - protein, 116 aa, synthetic construct
EVQLVESGGGLVQPGGSLRLSCAASGFTFSSYAMSWVRQAPGKGLEWVASINSGGFTYYPDSVKGRFTISRDNSKNTLYLQMNSLRAEDTAVYYCARGGTTVFDYWGQGTLVTVSS

SEQ ID NO: 25 - protein, 11 aa, synthetic construct
KASQDVSTAVA

SEQ ID NO: 26 - protein, 7 aa, synthetic construct
SASYRYT

SEQ ID NO: 27 - protein, 9 aa, synthetic construct
QQHFNTPRT

SEQ ID NO: 28 - protein, 5 aa, synthetic construct
SYAMS

SEQ ID NO: 29 - protein, 16 aa, synthetic construct
SINSGGFTYYPQSVQG

SEQ ID NO: 30 - protein, 8 aa, synthetic construct
GGTTVFDY

SEQ ID NO: 31 - protein, 107 aa, synthetic construct; identical to SEQ ID NO: 23

SEQ ID NO: 32 - protein, 116 aa, synthetic construct
QVKLVQSGAEVKKPGASLKVSCAASGFTFSSYAMSWVRQTPEQRLEWVASINSGGFTYYPQSVQGRFTISRDNTRNILYLELSSLRSEDTAMYYCARGGTTVFDYWGQGTTLTVSS

SEQ ID NO: 33 - protein, 11 aa, synthetic construct
RASQDVSTAVN

SEQ ID NO: 34 - protein, 7 aa, synthetic construct
SASYLYT

SEQ ID NO: 35 - protein, 9 aa, synthetic construct
QQHFNTPRT

SEQ ID NO: 36 - protein, 5 aa, synthetic construct
SYAMS

SEQ ID NO: 37 - protein, 16 aa, synthetic construct
SINSGGFTYYPDSVKG

SEQ ID NO: 38 - protein, 8 aa, synthetic construct
GGTTVFDY

SEQ ID NO: 39 - protein, 107 aa, synthetic construct
DIQMTQSHSFMSASVGDRVSITCRASQDVSTAVNWYQQKPGHAPKLLIYSASYLYTGVPDRFTGSGSGADFTFTISSLQAEDLAVYFCQQHFNTPRTFGGGTKLEIK

SEQ ID NO: 40 - protein, 116 aa, synthetic construct; identical to SEQ ID NO: 24

SEQ ID NO: 41 - protein, 11 aa, synthetic construct
RASQDVSTAVN

SEQ ID NO: 42 - protein, 7 aa, synthetic construct
SASYLYT

SEQ ID NO: 43 - protein, 9 aa, synthetic construct
QQHFNTPRT

SEQ ID NO: 44 - protein, 5 aa, synthetic construct
SYAMS

SEQ ID NO: 45 - protein, 16 aa, synthetic construct
SINSGGFTYYPQSVQG

SEQ ID NO: 46 - protein, 8 aa, synthetic construct
GGTTVFDY

SEQ ID NO: 47 - protein, 107 aa, synthetic construct; identical to SEQ ID NO: 39

SEQ ID NO: 48 - protein, 116 aa, synthetic construct; identical to SEQ ID NO: 32

SEQ ID NO: 49 - protein, 11 aa, synthetic construct
KASQNVGTNVA

SEQ ID NO: 50 - protein, 7 aa, synthetic construct
SASYRYS

SEQ ID NO: 51 - protein, 9 aa, synthetic construct
QQYNSYPLT

SEQ ID NO: 52 - protein, 5 aa, synthetic construct
SYAMS

SEQ ID NO: 53 - protein, 17 aa, synthetic construct
TISSGGSYTYYLDSVKG

SEQ ID NO: 54 - protein, 14 aa, synthetic construct
PPARAGIYYYVMDY

SEQ ID NO: 55 - protein, 107 aa, synthetic construct
DIVMTQSQKFMSTSVGDRVSVTCKASQNVGTNVAWYQQKPGQSPKPLIYSASYRYSGVPDRFTGSGSGTDFTLTISNVQSEDLAEYFCQQYNSYPLTFGAGTKLELK

SEQ ID NO: 56 - protein, 123 aa, synthetic construct
EVQLVESGGGLVKPGGSLKLSCAASGFTFSSYAMSWVRQTPEKRLEWVATISSGGSYTYYLDSVKGRFTISRDNAKNTLYLQMSSLRSEDTAMYYCARPPARAGIYYYVMDYWGQGTSVTVSS

SEQ ID NO: 57 - protein, 11 aa, synthetic construct
RASQDINNYLH

SEQ ID NO: 58 - protein, 7 aa, synthetic construct
YTSRLHS

SEQ ID NO: 59 - protein, 9 aa, synthetic construct
QQGKTLPWT

SEQ ID NO: 60 - protein, 5 aa, synthetic construct
DYGMH

SEQ ID NO: 61 - protein, 17 aa, synthetic construct
YISTGSGTIYYADTVKG

SEQ ID NO: 62 - protein, 9 aa, synthetic construct
QGYSYTMDY

SEQ ID NO: 63 - protein, 107 aa, synthetic construct
DVQMTQTTSSLSASLGDRVTISCRASQDINNYLHWYQQKPDGTVKLLIYYTSRLHSGVPSRFSGSGSGTDYSLTISNLDQEDIATYFCQQGKTLPWTFGGGTKLEIK

SEQ ID NO: 64 - protein, 118 aa, synthetic construct
DVQLVESGGGLVKPGGSRKLSCAASGFTFSDYGMHWVRQAPEKGLEWIAYISTGSGTIYYADTVKGRFTISRDNAKSTLFLQMTGLRSEDTAMYYCARQGYSYTMDYWGQGTSVTVSS

SEQ ID NO: 65 - protein, 11 aa, synthetic construct
KASQDVSTAVA

SEQ ID NO: 66 - protein, 7 aa, synthetic construct
SASYRST

SEQ ID NO: 67 - protein, 9 aa, synthetic construct
QQHFNTPRT

SEQ ID NO: 68 - protein, 5 aa, synthetic construct
SYAMS

SEQ ID NO: 69 - protein, 16 aa, synthetic construct
SINSGGFTYYPDSVKG

SEQ ID NO: 70 - protein, 8 aa, synthetic construct
GGTTVFDY

SEQ ID NO: 71 - protein, 107 aa, synthetic construct
DIVMTQSHKFMSTSVGDRVSITCKASQDVSTAVAWYQQRPGHSPKLLIYSASYRSTGVPDRFTGSGSGTDFTFTISSVQAEDLAVYYCQQHFNTPRTFGGGTKLEIK

SEQ ID NO: 72 - protein, 116 aa, synthetic construct
EVKLVESGGGLVKPGGSLKLSCAASGFTFSSYAMSWVRQTPEKRLEWVASINSGGFTYYPDSVKGRFTISRDNARNILYLQMSSLRSEDTAMYYCARGGTTVFDYWGQGTTLTVSS

SEQ ID NO: 73 - protein, 15 aa, synthetic construct
RASKSVSTSGYSYMH

SEQ ID NO: 74 - protein, 7 aa, synthetic construct
LVSNLES

SEQ ID NO: 75 - protein, 9 aa, synthetic construct
QQHFNTPRT

SEQ ID NO: 76 - protein, 5 aa, synthetic construct
SYAMS

SEQ ID NO: 77 - protein, 16 aa, synthetic construct
SINSGGFTYYPDSVKG

SEQ ID NO: 78 - protein, 8 aa, synthetic construct
GGTTVFDY

SEQ ID NO: 79 - protein, 111 aa, synthetic construct
DIVLTQSPASLAVSLGQRATISYRASKSVSTSGYSYMHWNQQKPGQPPRLLIYLVSNLESGVPARFSGSGSGTDFTLNIHPVEEEDAATYYCQQHFNTPRTFGGGTKLEIK

SEQ ID NO: 80 - protein, 116 aa, synthetic construct
EVQLQESGGGLVKPGGSLKLSCAASGFTFSSYAMSWVRQTPEKRLEWVASINSGGFTYYPDSVKGRFTISRDNARNILYLQMSSLRSEDTAMYYCARGGTTVFDYWGQGTTVTVSS

SEQ ID NO: 81 - protein, 17 aa, synthetic construct
KSSQSLLNSGNQKNYLT

SEQ ID NO: 82 - protein, 7 aa, synthetic construct
WTSTRES

SEQ ID NO: 83 - protein, 9 aa, synthetic construct
QNDYSYPFT

SEQ ID NO: 84 - protein, 5 aa, synthetic construct
SNWIE

SEQ ID NO: 85 - protein, 17 aa, synthetic construct
EILPGSGRANNNEKFKG

SEQ ID NO: 86 - protein, 10 aa, synthetic construct
PNLFGGAMDY

SEQ ID NO: 87 - protein, 113 aa, synthetic construct
DIVMTQSPSSLTVTAGEKVTMSCKSSQSLLNSGNQKNYLTWYQQKPGQPPKLLIYWTSTRESGVPARFTGSGSGTDFTLTISSVQAEDLAVYYCQNDYSYPFTFGSGTKLEIK

SEQ ID NO: 88 - protein, 119 aa, synthetic construct
QVQLQQSGAELMKPGASVKISCKATGYTISSNWIEWIKQRPGHGLEWIGEILPGSGRANNNEKFKGKASFTADTSSNTAYIQLSSLTSEDSAVYYCARPNLFGGAMDYWGQGTSVTVSS

SEQ ID NO: 89 - protein, 15 aa, synthetic construct
RASESVDTYGNSFMH

SEQ ID NO: 90 - protein, 7 aa, synthetic construct
RASNLES

SEQ ID NO: 91 - protein, 9 aa, synthetic construct
QQSNKDPWT

SEQ ID NO: 92 - protein, 5 aa, synthetic construct
SNWIE

SEQ ID NO: 93 - protein, 17 aa, synthetic construct
EILPGSGRANNNEKFKG

SEQ ID NO: 94 - protein, 10 aa, synthetic construct
PNLFGGAMDY

SEQ ID NO: 95 - protein, 111 aa, synthetic construct
DIVLTQSPASLAVSLGQRATISCRASESVDTYGNSFMHWYQQKPGQPPKLLIYRASNLESGIPARFSGSGSRTDFTLTINPVEADDVATYYCQQSNKDPWTFGGGTKLEIK

SEQ ID NO: 96 - protein, 119 aa, synthetic construct; identical to SEQ ID NO: 88

SEQ ID NO: 97 - protein, 11 aa, synthetic construct
KASQDVYTAVA

SEQ ID NO: 98 - protein, 7 aa, synthetic construct
SASYRYT

SEQ ID NO: 99 - protein, 9 aa, synthetic construct
QQHYSAPRT

SEQ ID NO: 100 - protein, 5 aa, synthetic construct
SYAMS

SEQ ID NO: 101 - protein, 16 aa, synthetic construct
SINSGGITYSPDSVKG

SEQ ID NO: 102 - protein, 8 aa, synthetic construct
GGTTFFDY

SEQ ID NO: 103 - protein, 107 aa, unidentified
DIVMTQSHKFMSTSVGDRVSISCKASQDVYTAVAWYQQKPGQSPKLLIYSASYRYTGVPDRFTGSGSGTDFTFTISTVQAEDLAVYYCQQHYSAPRTFGGGTKLEIK

SEQ ID NO: 104 - protein, 116 aa, synthetic construct
EVKLMESGGGLVKPGGSLKLSCAASGFTFSSYAMSWVRQTPEKRLEWVASINSGGITYSPDSVKGRFTISRDNARNILYLQMSSLRSEDAAIYFCARGGTTFFDYWGQGTTLTVSS

SEQ ID NO: 105 - protein, 11 aa, synthetic construct
KASQDVSTTVA

SEQ ID NO: 106 - protein, 7 aa, synthetic construct
WASTRHT

SEQ ID NO: 107 - protein, 9 aa, synthetic construct
QQHYSTPRT

SEQ ID NO: 108 - protein, 5 aa, synthetic construct
SYAMS

SEQ ID NO: 109 - protein, 16 aa, synthetic construct
SINSGGITYYPDSVKG

SEQ ID NO: 110 - protein, 8 aa, synthetic construct
GGTTVFDY

SEQ ID NO: 111 - protein, 107 aa, synthetic construct
DIVMTQSHKFMSTSVGDRVSITCKASQDVSTTVAWYQQKPGQSPKLLIYWASTRHTGVPDRFTGSGSGTDYTLTISPVQAEDLALYYCQQHYSTPRTFGGGTKLEIK

SEQ ID NO: 112 - protein, 116 aa, synthetic construct
EMKLVESGGGLIKPGGSLKLSCAASGFTFSSYAMSWVRQTPEKRLEWVASINSGGITYYPDSVKGRFTISRDNARNILYLQMSSLRSEDTAMYYCARGGTTVFDYWGQGTTLTVSS

SEQ ID NO: 113 - protein, 11 aa, synthetic construct
KASQDVSSAVA

SEQ ID NO: 114 - protein, 7 aa, synthetic construct
WASTRHT

SEQ ID NO: 115 - protein, 9 aa, synthetic construct
QQHFSSPRT

SEQ ID NO: 116 - protein, 5 aa, synthetic construct
SYAMS

SEQ ID NO: 117 - protein, 16 aa, synthetic construct
SLNSGGITYYPDSVRG

SEQ ID NO: 118 - protein, 8 aa, synthetic construct
GGTTVFDY

SEQ ID NO: 119 - protein, 107 aa, synthetic construct
DIVLTQSHKFMSTSVGDRVNITCKASQDVSSAVAWYQQKPGQSPKLLIYWASTRHTGVPDRFTGSGSGTDYTLTISTVQAEDLALYYCQQHFSSPRTFGGGTKLEIK

SEQ ID NO: 120 - protein, 116 aa, synthetic construct
EVKLVESGGGLVRPGGSLKLSCAASGFSFSSYAMSWVRQTPEKRLDWVASLNSGGITYYPDSVRGRFTISRDIVRHTLYLQMSSLRSEDTAMYFCARGGTTVFDYWGQGTTLTVSS

SEQ ID NO: 121 - protein, 10 aa, synthetic construct
SASSSISYIY

SEQ ID NO: 122 - protein, 7 aa, synthetic construct
DTSNLAS

SEQ ID NO: 123 - protein, 10 aa, synthetic construct
QQWSSYPPIT

SEQ ID NO: 124 - protein, 5 aa, synthetic construct
AYTMN

SEQ ID NO: 125 - protein, 17 aa, synthetic construct
LINPYNGDTVFNQKFKD

SEQ ID NO: 126 - protein, 10 aa, synthetic construct
GLRTYWYFDV

SEQ ID NO: 127 - protein, 107 aa, synthetic construct
QIVLTQSPAIMSASPGEKVTMTCSASSSISYIYWYQQKPGSSPRLLIYDTSNLASGVPVRFSGSGSGTSYSLTISRVEAEDAATYYCQQWSSYPPITFGGGTKLEIK

SEQ ID NO: 128 - protein, 117 aa, synthetic construct
QLQQSGPEPVKPGTSMKISCKASGYSFTAYTMNWVKQSHGKTLEWIGLINPYNGDTVFNQKFKDKATLTVDRSSSTAFMELLSLTSDDSAVYYCARGLRTYWYFDVWGAGTTITVSS

SEQ ID NO: 129 - DNA, 51 bp, synthetic construct
  source 1..51: mol_type = other DNA
aaaagctctcaatccttgctgaacagcggaaaccaaaagaagtacttgaca

SEQ ID NO: 130 - DNA, 21 bp, synthetic construct
  source 1..21: mol_type = other DNA
tgggctgcaacacgggagagc

SEQ ID NO: 131 - DNA, 27 bp, synthetic construct
  source 1..27: mol_type = other DNA
cagaacgactacacctatcctctgaca

SEQ ID NO: 132 - DNA, 15 bp, synthetic construct
  source 1..15: mol_type = other DNA
aattactggattgaa

SEQ ID NO: 133 - DNA, 51 bp, synthetic construct
  source 1..51: mol_type = other DNA
gaaatatttccagggagcgggacgacaaagtataacgaaaagttcaaaggc

SEQ ID NO: 134 - DNA, 36 bp, synthetic construct
  source 1..36: mol_type = other DNA
ggcaggtttatcagtaccacaaggggattcgcctat

SEQ ID NO: 135 - DNA, 339 bp, synthetic construct
  source 1..339: mol_type = other DNA
gacatcgtgatgacccaatcaccaagtagcctgactgtgacgcccggggaaaaggtcaccatgtcctgtaaaagctctcaatccttgctgaacagcggaaaccaaaagaagtacttgacatggtaccaacagaaacccggtcaaccaccaaagctcttgatatattgggctgcaacacgggagagcggtgttccagacaggttcaccggcagtggatctggcaccgattttacccttacgatcagctccgttcaggctgaagacttagctgtttactattgtcagaacgactacacctatcctctgacatttggggccgggacaaaattagagcttaag

SEQ ID NO: 136 - DNA, 363 bp, synthetic construct
  source 1..363: mol_type = other DNA
caagttcaactgcaacaatccggcgcagaactgacaaaacctggggcatccgtcaaaattagctgtaaagcgactgggtatactttctcaaattactggattgaatgggttattcagcggccaggtcatggccttgaatggattggcgaaatatttccagggagcgggacgacaaagtataacgaaaagttcaaaggcaaagccacattcacagccgacacgtctagtaatactgcgtatatgcaattaaatagtttaacctcagaagattccgcggtgtattattgcgcacggggcaggtttatcagtaccacaaggggattcgcctattggggccaagggacattggtaacagtaagtgcc

SEQ ID NO: 137 - DNA, 33 bp, synthetic construct
  source 1..33: mol_type = other DNA
aaagcttctcaggatgtaagtacagccgtcgcg

SEQ ID NO: 138 - DNA, 21 bp, synthetic construct
  source 1..21: mol_type = other DNA
tccgcgagctatcgatatact

SEQ ID NO: 139 - DNA, 27 bp, synthetic construct
  source 1..27: mol_type = other DNA
caacaacactttaatacaccacgaacc

SEQ ID NO: 140 - DNA, 15 bp, synthetic construct
  source 1..15: mol_type = other DNA
agttatgcgatgtca

SEQ ID NO: 141 - DNA, 48 bp, synthetic construct
  source 1..48: mol_type = other DNA
tctattaatagtggcggttttacctactatcctgacagtgtcaaaggc

SEQ ID NO: 142 - DNA, 24 bp, synthetic construct
  source 1..24: mol_type = other DNA
ggcgggacaaccgtcttcgactac

SEQ ID NO: 143 - DNA, 321 bp, synthetic construct
  source 1..321: mol_type = other DNA
gatattgttatgactcaatcccacaaatttatgtctacaagtgtaggcgacagggtcagcatcacctgcaaagcttctcaggatgtaagtacagccgtcgcgtggtatcaacagaaacctggacacagcccaaagttgctgatatactccgcgagctatcgatatactggtgtgcccgacaggtttacagggtcaggtagcggcgcggatttcacgtttacaatcagctctgtacaggcagaagacttagccgtgtacttttgtcaacaacactttaatacaccacgaacctttggcggcggtaccaaactggagataaag

SEQ ID NO: 144 - DNA, 348 bp, synthetic construct
  source 1..348: mol_type = other DNA
gaagtgaaacttgtggagtctggtggtggattggttaagcccggtggtagcttgaaactgtcctgcgcagcgtctggctttactttctccagttatgcgatgtcatgggtgcgccaaacacctgagaagaggctggagtgggtagcctctattaatagtggcggttttacctactatcctgacagtgtcaaaggcaggtttacaatttctcgcgacaacaccagaaatatcttgtatcttcaactcagtagcttgaggtctgaagacacggcaatgtattactgtgcccgaggcgggacaaccgtcttcgactactggggacaagggacgaccttgacggtctcatca

SEQ ID NO: 145 - DNA, 33 bp, synthetic construct
  source 1..33: mol_type = other DNA
aaagccagtcagaacgtaggaaccaatgtcgcc

SEQ ID NO: 146 - DNA, 21 bp, synthetic construct
  source 1..21: mol_type = other DNA
tcagccagctatcgatattcc

SEQ ID NO: 147 - DNA, 27 bp, synthetic construct
  source 1..27: mol_type = other DNA
caacaatataacagctaccccctcaca

SEQ ID NO: 148 - DNA, 15 bp, synthetic construct
  source 1..15: mol_type = other DNA
tcttatgcaatgagt

SEQ ID NO: 149 - DNA, 51 bp, synthetic construct
  source 1..51: mol_type = other DNA
accatatcaagcggtggaagttacacatattacttagacagtgtgaaagga

SEQ ID NO: 150 - DNA, 42 bp, synthetic construct
  source 1..42: mol_type = other DNA
ccacctgcacgggccggtatctattactacgtgatggactat

SEQ ID NO: 151 - DNA, 321 bp, synthetic construct
  source 1..321: mol_type = other DNA
gacatcgtcatgacacagtcacagaaatttatgtctacatcagtgggcgatcgggttagcgtcacctgtaaagccagtcagaacgtaggaaccaatgtcgcctggtatcaacagaaacctggccaaagtcccaaacctcttatttactcagccagctatcgatattccggagtcccagatcggtttactggaagtggaagcggaactgactttaccctcactattagtaacgttcaatcagaagatttagccgagtatttctgccaacaatataacagctaccccctcacatttggggcaggcactaagttagagttgaag

SEQ ID NO: 152 - DNA, 369 bp, synthetic construct
  source 1..369: mol_type = other DNA
gaagtacaacttgttgagtctggcggcggtctggttaaacctggcggatcccttaaattgagttgtgccgcttccgggttcacgttcagctcttatgcaatgagttgggtccgtcagaccccggaaaagcgcctcgaatgggtcgccaccatatcaagcggtggaagttacacatattacttagacagtgtgaaaggacgattcaccatttcaagagacaacgctaagaacactttgtatttacaaatgtctagcttaaggtcagaagatactgccatgtattattgtgcaagaccacctgcacgggccggtatctattactacgtgatggactattggggccaaggaacgagcgtcacagtgtcatca

SEQ ID NO: 153 - DNA, 33 bp, synthetic construct
  source 1..33: mol_type = other DNA
cgcgcaagtcaggacatcaataattatctgcac

SEQ ID NO: 154 - DNA, 21 bp, synthetic construct
  source 1..21: mol_type = other DNA
tacacatcaaggctgcatagc

SEQ ID NO: 155 - DNA, 27 bp, synthetic construct
  source 1..27: mol_type = other DNA
caacaaggaaagacactgccgtggacc

SEQ ID NO: 156 - DNA, 15 bp, synthetic construct
  source 1..15: mol_type = other DNA
gattacgggatgcat

SEQ ID NO: 157 - DNA, 51 bp, synthetic construct
  source 1..51: mol_type = other DNA
tatatttccactggaagtggtacgatttattatgcggatacagtcaagggt

SEQ ID NO: 158 - DNA, 27 bp, synthetic construct
  source 1..27: mol_type = other DNA
cagggatacagctatacaatggactat

SEQ ID NO: 159 - DNA, 321 bp, synthetic construct
  source 1..321: mol_type = other DNA
gacgttcaaatgacccaaaccacttccagcttgtccgcctctctgggtgatagagtaacgatctcctgccgcgcaagtcaggacatcaataattatctgcactggtatcaacaaaagccggatggaacagtaaagctgcttatatactacacatcaaggctgcatagcggcgtccccagcaggttctcaggatcagggtccgggacagactactctcttaccatatcaaacctcgaccaagaagatatcgcaacatacttctgccaacaaggaaagacactgccgtggaccttcggaggaggaacaaaactcgaaatcaaa

SEQ ID NO: 160 - DNA, 354 bp, synthetic construct
  source 1..354: mol_type = other DNA
gatgtacaattggtggaaagtggaggaggcttggtcaaaccgggtggaagtaggaaactcagctgcgccgcttcaggatttacattctccgattacgggatgcattgggttcgccaggccccagaaaagggccttgagtggatcgcgtatatttccactggaagtggtacgatttattatgcggatacagtcaagggtaggttcaccatttcccgcgacaacgcaaagagcaccctctttcttcaaatgaccggcttgagaagcgaggacaccgccatgtattattgcgccaggcagggatacagctatacaatggactattggggacaaggaacgagtgttacagttagtagt

SEQ ID NO: 161 - DNA, 33 bp, synthetic construct
  source 1..33: mol_type = other DNA
aaagcatctcaagacgtctccacagctgtagcc

SEQ ID NO: 162 - DNA, 21 bp, synthetic construct
  source 1..21: mol_type = other DNA
agtgctagttacaggtccaca

SEQ ID NO: 163 - DNA, 27 bp, synthetic construct
  source 1..27: mol_type = other DNA
cagcagcactttaacacccctcggacg

SEQ ID NO: 164 - DNA, 15 bp, synthetic construct
  source 1..15: mol_type = other DNA
agctatgcgatgtca

SEQ ID NO: 165 - DNA, 48 bp, synthetic construct
  source 1..48: mol_type = other DNA
tctataaactcaggtgggtttacctattacccagattcagttaaaggt

SEQ ID NO: 166 - DNA, 24 bp, synthetic construct
  source 1..24: mol_type = other DNA
ggtgggaccaccgttttcgactac

SEQ ID NO: 167 - DNA, 321 bp, synthetic construct
  source 1..321: mol_type = other DNA
gatatcgtcatgacccaaagccacaaatttatgtccaccagtgtcggggacagagtgagtattacttgtaaagcatctcaagacgtctccacagctgtagcctggtatcaacaacggcccggtcatagccccaaactgctcatttacagtgctagttacaggtccacaggagtacctgatcgctttacgggcagtgggtcaggcactgatttcacttttactatctcatccgtgcaggctgaggacttggcggtatattactgccagcagcactttaacacccctcggacgttcggcggtgggaccaaactggagatcaaa

SEQ ID NO: 168 - DNA, 348 bp, synthetic construct
  source 1..348: mol_type = other DNA
gaagtcaaactcgtagaaagcggcggcggcctcgtaaaacccggcgggtcactgaagctcagttgtgcggcgtcaggattcacattctcaagctatgcgatgtcatgggtcaggcaaactccagagaaaagattggaatgggtggcatctataaactcaggtgggtttacctattacccagattcagttaaaggtcggtttacaatttccagggacaatgcgcggaatatcctgtacctgcaaatgagcagcctgcgcagcgaagacacggcaatgtactactgtgctcgcggtgggaccaccgttttcgactactggggacaagggactacactgacagtttctagt

SEQ ID NO: 169 - DNA, 45 bp, synthetic construct
  source 1..45: mol_type = other DNA
cgagcctctaaaagcgtgagcacgagcggctatagctacatgcac

SEQ ID NO: 170 - DNA, 21 bp, synthetic construct
  source 1..21: mol_type = other DNA
ctggtctccaatttggagtcc

SEQ ID NO: 171 - DNA, 27 bp, synthetic construct
  source 1..27: mol_type = other DNA
caacaacacttcaataccccgcgcacc

SEQ ID NO: 172 - DNA, 15 bp, synthetic construct
  source 1..15: mol_type = other DNA
tcatatgcaatgtca

SEQ ID NO: 173 - DNA, 48 bp, synthetic construct
  source 1..48: mol_type = other DNA
agtatcaattccggcggtttcacttattaccctgattctgtgaagggt

SEQ ID NO: 174 - DNA, 24 bp, synthetic construct
  source 1..24: mol_type = other DNA
ggtggaactaccgtcttcgattac

SEQ ID NO: 175 - DNA, 333 bp, synthetic construct
  source 1..333: mol_type = other DNA
gatatcgtgctgactcaatctcctgcctctttagcggttagcctgggccaaagggcaactatctcttaccgagcctctaaaagcgtgagcacgagcggctatagctacatgcactggaatcaacagaaacctggccaaccgcccagactcttgatttatctggtctccaatttggagtccggtgtcccggcccgattctcaggatccggtagcgggactgattttacgctcaacatacatcctgttgaagaggaagacgctgccacttattactgtcaacaacacttcaataccccgcgcaccttcggcggcggcactaaactcgagatcaag

SEQ ID NO: 176 - DNA, 348 bp, synthetic construct
  source 1..348: mol_type = other DNA
gaggtccaattacaagagagtggtggcggattggttaaaccaggtggatctttaaagcttagctgtgccgcaagcggctttaccttttcatcatatgcaatgtcatgggtgcgacaaacccccgagaaacgtttggaatgggttgccagtatcaattccggcggtttcacttattaccctgattctgtgaagggtcgctttaccatctctagagataacgctagaaacatcttgtacctgcaaatgagtagcctgcgtagtgaagacactgcaatgtactattgcgcccgcggtggaactaccgtcttcgattactggggtcaaggtaccactgtgacagtatcatca

SEQ ID NO: 177 - DNA, 51 bp, synthetic construct
  source 1..51: mol_type = other DNA
aaatctagccaatctcttttaaattctggaaaccagaagaactatctgaca

SEQ ID NO: 178 - DNA, 15 bp, synthetic construct
  source 1..15: mol_type = other DNA
tggacaagcacaagg

SEQ ID NO: 179 - DNA, 27 bp, synthetic construct
  source 1..27: mol_type = other DNA
caaaacgattactcttaccctttcacg

SEQ ID NO: 180 - DNA, 15 bp, synthetic construct
  source 1..15: mol_type = other DNA
tcaaactggattgag

SEQ ID NO: 181 - DNA, 51 bp, synthetic construct
  source 1..51: mol_type = other DNA
gagattcttccaggaagtgggcgggcaaacaacaatgagaaatttaagggc

SEQ ID NO: 182 - DNA, 30 bp, synthetic construct
  source 1..30: mol_type = other DNA
cccaacctctttggtggagctatggattat

SEQ ID NO: 183 - DNA, 339 bp, synthetic construct
  source 1..339: mol_type = other DNA
gatatcgtgatgacacaaagtccttcatctctgacagttacagctggggagaaagtcaccatgtcctgtaaatctagccaatctcttttaaattctggaaaccagaagaactatctgacatggtaccaacagaaaccgggccaacccccaaagctgctcatttattggacaagcacaagggagagcggggttccagcgagatttacagggtctggctcaggaacggatttcacactgaccatctcatccgtgcaagctgaagacctggcagtttactactgtcaaaacgattactcttaccctttcacgttcggcagcggaaccaagttggaaatcaaa

SEQ ID NO: 184 - DNA, 357 bp, synthetic construct
  source 1..357: mol_type = other DNA
caagtacagctgcaacaatcaggcgcggagctgatgaaacccggtgccagcgtcaagatttcttgcaaagccaccggctatacaattagctcaaactggattgagtggattaaacaacggcccgggcacggactcgaatggataggagagattcttccaggaagtgggcgggcaaacaacaatgagaaatttaagggcaaggcatccttcaccgcagatacgtcttccaacacggcttacattcaattgagctctctcacatctgaagactccgccgtatattattgcgcccggcccaacctctttggtggagctatggattattggggccaggggaccagcgtgaccgttagcagc

SEQ ID NO: 185 - DNA, 45 bp, synthetic construct
  source 1..45: mol_type = other DNA
cgggcctctgaatcagtagacacttacgggaacagttttatgcac

SEQ ID NO: 186 - DNA, 21 bp, synthetic construct
  source 1..21: mol_type = other DNA
agagcttccaacctcgaatcc

SEQ ID NO: 187 - DNA, 27 bp, synthetic construct
  source 1..27: mol_type = other DNA
cagcaaagcaacaaagacccgtggaca

SEQ ID NO: 188 - DNA, 15 bp, synthetic construct
  source 1..15: mol_type = other DNA
tcaaactggattgag

SEQ ID NO: 189 - DNA, 51 bp, synthetic construct; identical to SEQ ID NO: 181
  source 1..51: mol_type = other DNA

SEQ ID NO: 190 - DNA, 30 bp, synthetic construct; identical to SEQ ID NO: 182
  source 1..30: mol_type = other DNA

SEQ ID NO: 191 - DNA, 333 bp, synthetic construct
  source 1..333: mol_type = other DNA
gatatcgttttgacacaaagtccagccagtctcgccgtttccttgggccaacgggctactattagttgccgggcctctgaatcagtagacacttacgggaacagttttatgcactggtatcaacagaaacccggccagcccccaaaactcttgatatacagagcttccaacctcgaatccgggatcccggctcggtttagtgggtccggatccagaacagatttcactcttaccatcaatcctgttgaggccgatgatgttgccacctactactgccagcaaagcaacaaagacccgtggacattcggcggtgggaccaaactggaaataaag

SEQ ID NO: 192 - DNA, 357 bp, synthetic construct; identical to SEQ ID NO: 184
  source 1..357: mol_type = other DNA

SEQ ID NO: 193 - protein, 238 aa, synthetic construct
MGWSCIILFLVATATGVHSDVVMTQSPLSLPVTPGEPASISCRSSQSLLHSNGNNYLDWYLQKPGQSPQLLIYLGSNRASGVPDRFSGSGSDTDFTLQISRVEAEDVGVYYCMQGTHPAISFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 194 - protein, 468 aa, synthetic construct
MGWSCIILFLVATATGVHSEVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGVGAHDAFDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - DNA, 717 bp, synthetic construct
  source 1..717: mol_type = other DNA
atgggatggtcatgtatcatcctttttctagtagcaactgcaactggagtacattcagatgttgtgatgactcagtctccactctccctgcccgtcacccctggagagccggcctccatctcctgcaggtctagtcagagcctcctgcatagtaatggaaacaactatttggattggtacctgcagaagccagggcagtctccacagctcctgatctatttgggttctaatcgggcctctggggtcccagacagattcagcggcagtgggtcagacactgatttcacactgcaaatcagtagggtggaggctgaggatgttggggtttattactgcatgcaaggtacacaccccgccatctccttcggccaagggacacgactggagattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 196 - DNA, 1411 bp, synthetic construct
  source 1..1411: mol_type = other DNA
atgggatggtcatggtatcatcctttttctagtagcaactgcaactggagtacattcagaggtccagctggtgcagtctgggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgaggggagtgggagcgcatgatgcttttgatatctggggccaagggaccacggtcaccgtctcaagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacagcacgtaccgtgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaatgagg

(19) United States
(12) Patent Application Publication
(10) Pub. No.: US 2026/0159608 A1
(43) Pub. Date: Jun. 11, 2026

(54) FLT3-BINDING ANTIBODIES AND METHODS OF USE THEREOF

(71) Applicant: Antharis Therapeutics Inc., San Diego, CA (US)

(72) Inventors: Raphael RIBEIRO-PINAUD, San Diego, CA (US); Jinming XIA, San Diego, CA (US); Eric SJOBERG, San Diego, CA (US); Robert HOFF, San Diego, CA (US); Seyedehsareh SEYEDI, San Diego, CA (US); Jerome HAN, San Diego, CA (US); Sai SURAPARAJU, San Diego, CA (US); Dominic KOWALSKI, San Diego, CA (US); Mary DWYER, San Diego, CA (US); Daniel YAZBECK, San Diego, CA (US)

(21) Appl. No.: 19/369,013

(22) Filed: Oct. 24, 2025

Related U.S. Application Data
(60) Provisional application No. 63/712,327, filed on Oct. 25, 2024.
(60) Provisional application No. 63/826,296, filed on Jun. 18, 2025.

(51) Int. Cl. C07K 16/40; A61K 31/4745; A61K 38/08; A61K 45/06; A61P 35/02; C12N 15/11

(52) U.S. Cl. CPC C07K 16/40; A61K 31/4745; A61K 38/08; A61K 45/06; A61P 35/02; C12N 15/11; C07K 2317/24; C07K 2317/34; C07K 2317/55; C07K 2317/565; C07K 2317/622

(57) ABSTRACT

Provided herein are, inter alia, novel antibodies that bind to fms-like tyrosine kinase 3 (FLT3) thereby effectively targeting cells expressing FLT3. The antibodies provided herein may be used, inter alia, for therapeutic cancer applications, including, in some embodiments, treatment of multiple cancer types, which may include acute myeloid leukemia (AML), lymphoblastic leukemia (ALL), lung cancer, breast cancer, pancreatic cancer, ovarian cancer, colorectal cancer, renal cancer, or glioblastoma.

### CROSS-REFERENCE TO RELATED APPLICATIONS

This application claims the benefit of priority to U.S. Provisional Patent Application Ser. No. 63/712,327, filed Oct. 25, 2024 and U.S. Provisional Patent Application Ser. No. 63/826,296, filed Jun. 18, 2025, which are hereby incorporated by reference in their entireties.

### REFERENCE TO A “SEQUENCE LISTING,” A TABLE, OR A COMPUTER PROGRAM LISTING APPENDIX SUBMITTED ON A COMPACT DISK

The Sequence Listing written in file 057868-510P02US_SequenceListing_ST26.xml, created on May 23, 2025, 189,820 bytes, machine format IBM-PC, MS-Windows operating system, is hereby incorporated by reference in its entirety for all purposes.

### BACKGROUND

Flt3 is a receptor tyrosine kinase with expression and function restricted to hematopoietic precursors where it promotes proliferation, survival, and the stem cell phenotype. Flt3 has also been found to be overexpressed in acute myeloid leukemia (AML).

ADCs are a growing class of biologics that have found growing therapeutics relevance in oncology indications. In the ADC modality a cytotoxic drug (payload) is conjugated to a monoclonal antibody via a chemical linker. This mechanism necessitates the antibody is internalized into the target cell following binding to the antigen. The ADC is co-trafficked with the target antigen into the cell to elicit its cytotoxic effect. Critical to the success of the ADC is the specificity and expression of the tumor associated antigen (TAA) targeted by the monoclonal antibody. Development of antibodies useful in treating AML and ALL would be beneficial to clinical outcomes.

### BRIEF SUMMARY

Disclosed herein are anti-fms-like tyrosine kinase 3 (FLT3) antibodies comprising a light chain variable domain and a heavy chain variable domain, wherein the light chain variable domain comprises: a light chain CDR1 (CDR L1) having at least 90% sequence identity to a light chain CDR1 amino acid sequence selected from Table 2, a light chain CDR2 (CDR L2) having at least 90% sequence identity to a light chain CDR2 amino acid sequence selected from Table 2, and a light chain CDR3 (CDR L3) having at least 90% sequence identity to a light chain CDR3 amino acid sequence selected from Table 2; and wherein the heavy chain variable domain comprises: a heavy chain CDR1 having at least 90% sequence identity to a heavy chain CDR1 (CDR H1) amino acid sequence selected from Table 2, a heavy chain CDR2 (CDR H2) having at least 90% sequence identity to a heavy chain CDR2 amino acid sequence selected from Table 2, and a heavy chain CDR3 (CDR H3) having at least 90% sequence identity to a heavy chain CDR3 amino acid sequence selected from Table 2. Disclosed herein are an anti-fms-like tyrosine kinase 3 (FLT3) antibodies comprising a light chain variable domain and a heavy chain variable domain, e.g., wherein the light chain variable domain comprises: a light chain CDR1 as set forth in SEQ ID NO:9, a light chain CDR2 as set forth in SEQ ID NO: 10 and a light chain CDR3 as set forth in SEQ ID NO: 11; and wherein the heavy chain variable domain comprises: a heavy chain CDR1 as set forth in SEQ ID NO:12, a heavy chain CDR2 as set forth in SEQ ID NO:13, and a heavy chain CDR3 as set forth in SEQ ID NO: 14, wherein the light chain variable domain comprises: a light chain CDR1 as set forth in SEQ ID NO:17, a light chain CDR2 as set forth in SEQ ID NO:18 and a light chain CDR3 as set forth in SEQ ID NO:19; and wherein the heavy chain variable domain comprises: a heavy chain CDR1 as set forth in SEQ ID NO:20, a heavy chain CDR2 as set forth in SEQ ID NO:21, and a heavy chain CDR3 as set forth in SEQ ID NO:22, wherein the light chain variable domain comprises: a light chain CDR1 as set forth in SEQ ID NO:25, a light chain CDR2 as set forth in SEQ ID NO:26 and a light chain CDR3 as set forth in SEQ ID NO:27; and wherein the heavy chain variable domain comprises: a heavy chain CDR1 as set forth in SEQ ID NO:28, a heavy chain CDR2 as set forth in SEQ ID NO:29, and a heavy chain CDR3 as set forth in SEQ ID NO:30, wherein the light chain variable domain comprises: a light chain CDR1 as set forth in SEQ ID NO:33, a light chain CDR2 as set forth in SEQ ID NO:34 and a light chain CDR3 as set forth in SEQ ID NO:35; and wherein the heavy chain variable domain comprises: a heavy chain CDR1 as set forth in SEQ ID NO:36, a heavy chain CDR2 as set forth in SEQ ID NO:37, and a heavy chain CDR3 as set forth in SEQ ID NO:38, wherein the light chain variable domain comprises: a light chain CDR1 as set forth in SEQ ID NO:41, a light chain CDR2 as set forth in SEQ ID NO:42 and a light chain CDR3 as set forth in SEQ ID NO:43; and wherein the heavy chain variable domain comprises: a heavy chain CDR1 as set forth in SEQ ID NO:44, a heavy chain CDR2 as set forth in SEQ ID NO:45, and a heavy chain CDR3 as set forth in SEQ ID NO:46, or wherein the light chain variable domain as set forth in SEQ ID NO:193 and a heavy chain variable domain as set forth in SEQ ID NO:194. In some embodiments, the light chain variable domain comprises an amino acid sequence with at least 80%, 85%, 90%, 95%, 98%, 99%, or 100% sequence identity with a light chain variable domain sequence selected from Table 2. In some embodiments, the light chain variable domain comprises an amino acid sequence with at least 80%, 85%, 90%, 95%, 98%, 99%, or 100% sequence identity with a heavy chain variable domain sequence selected from Table 2. In some embodiments, the antibody is a humanized antibody. In some embodiments, the antibody is a chimeric antibody. In some embodiments, the antibody is an IgG. In some embodiments, the antibody is an IgG1. In some embodiments, the antibody is a Fab′ fragment. In some embodiments, the antibody is a single chain antibody (scFv). In some embodiments, the light chain variable domain and the heavy chain variable domain form part of a scFv. In some embodiments, the antibody is capable of binding FLT3. In some embodiments, the antibody is bound to FLT3. In some embodiments, the FLT3 forms part of a cell. In embodiments, the cell is a cancer cell. In embodiments, the cancer cell is a leukemia cell (an acute myeloid leukemia (AML) cell or an acute lymphoblastic leukemia (ALL) cell), a gastrointestinal cancer cell, a colorectal cancer cell, a stomach cancer cell, a pancreas cancer cell, a neuroendocrine cancer cell, or an ovarian cancer cell. In embodiments, the cancer cell is a gastrointestinal cancer cell. In embodiments, the cancer cell is a colorectal cancer cell. In embodiments, the cancer cell is a stomach cancer cell. In embodiments, the cancer cell is a pancreas cancer cell. In embodiments, the cancer cell is a neuroendocrine cancer cell. In embodiments, the cancer cell is an ovarian cancer cell. In some embodiments, the cell is selected from an acute myeloid leukemia cell, a gastrointestinal cancer cell, a colorectal cancer cell, a stomach cancer cell, a pancreas cancer cell, a neuroendocrine cancer cell, or an ovarian cancer cell. In some embodiments, the myeloid cell is a macrophage. In some embodiments, the anti-FLT3 antibody binds the same epitope as an antibody comprising: a heavy chain variable region domain comprising a CDR1 (CDR H1) as set forth in Table 2, a CDR2 (CDR H2) as set forth in Table 2, and a CDR3 (CDR H3) as set forth in Table 2, and a light chain variable domain comprising a CDR1 (CDR L1) as set forth in Table 2, a CDR2 (CDR L2) as set forth in Table 2, and a CDR3 (CDR L3) as set forth in Table 2. In some embodiments, the light chain variable domain comprises an amino acid sequence with at least 80%, 85%, 90%, 95%, 98%, 99%, or 100% sequence identity with a light chain variable domain amino acid sequence as set forth in Table 2. In some embodiments, the heavy chain variable domain comprises an amino acid sequence with at least 80%, 85%, 90%, 95%, 98%, 99%, or 100% sequence identity with a heavy chain variable domain amino acid sequence as set forth in Table 2.

In various embodiments, an isolated nucleic acid encodes an antibody described herein. In various embodiments, an isolated nucleic acid encoding a light chain variable domain and a heavy chain variable domain, wherein the portion of the nucleic acid encoding the light chain variable domain comprises: a CDR L1 nucleotide sequence having at least 90% sequence identity to a light chain CDR L1 nucleotide sequence selected from Table 2, a CDR L2 nucleotide sequence having at least 90% sequence identity to a light chain CDR L2 nucleotide sequence selected from Table 2, and a light chain CDR L3 nucleotide sequence having at least 90% sequence identity to a light chain CDR L3 nucleotide sequence selected from Table 2; and wherein the portion of the nucleic acid encoding the heavy chain variable domain comprises: a CDR H1 nucleotide sequence having at least 90% sequence identity to a heavy chain CDR H1 nucleotide sequence selected from Table 2, a CDR H2 nucleotide sequence having at least 90% sequence identity to a heavy chain CDR H2 nucleotide sequence selected from Table 2, and a CDR H3 nucleotide sequence having at least 90% sequence identity to a heavy chain CDR H3 nucleotide sequence selected from Table 2. In some embodiments, the isolated nucleic acid comprises a light chain variable domain sequence and a heavy chain variable domain sequence, wherein the light chain variable domain sequence comprises: a CDR1 as set forth in SEQ ID NO:153, a CDR2 as set forth in SEQ ID NO: 154 and a CDR3 as set forth in SEQ ID NO: 155; or wherein the heavy chain variable domain sequence comprises: a CDR1 as set forth in SEQ ID NO:156, a CDR2 as set forth in SEQ ID NO: 157, and a CDR3 as set forth in SEQ ID NO:158. In some embodiments, the light chain variable domain sequence comprises a nucleic acid sequence with at least 80%, 85%, 90%, 95%, 98%, 99%, or 100% sequence identity with a light chain variable domain nucleic acid sequence selected from Table 2. In some embodiments, the heavy chain variable domain comprises a nucleic acid sequence with at least 80%, 85%, 90%, 95%, 98%, 99%, or 100% sequence identity with a heavy chain variable domain nucleic acid sequence selected from Table 2. In various embodiments, a cell comprises an isolated nucleic acid described herein.

In various embodiments, a pharmaceutical composition comprises a therapeutically effective amount of an antibody described herein and a pharmaceutically acceptable excipient. In some embodiments, the pharmaceutical composition is formulated in an aqueous solution at a concentration of 0.200 mg/mL to 2.00 mg/mL, 0.500 mg/mL to 1.50 mg/mL, 0.700 mg/mL to 0.875 mg/mL, or 0.875 mg/mL to 1.25 mg/mL. In some embodiments, the pharmaceutical composition is formulated in an aqueous solution at a concentration of 1.25 mg/mL to 2.50 mg/mL, 2.50 mg/mL to 5.00 mg/mL, 5.00 mg/mL to 10.0 mg/mL, 10.0 mg/mL to 25.0 mg/mL, 25.0 mg/mL to 50.0 mg/mL, 50.0 mg/mL to 100 mg/mL, 100 mg/mL to 150 mg/mL, or 150 mg/mL to 200 mg/mL. In some embodiments, the pharmaceutical composition is formulated for intravenous delivery. In some embodiments, the pharmaceutical composition further comprises a therapeutically effective amount of an antineoplastic agent.

In various embodiments, a method of treating a subject in need thereof comprises administering to a subject a therapeutically effective amount of an antibody described herein. In some embodiments, the method further comprises administering a therapeutically effective amount of an antineoplastic agent or a pharmaceutical composition. In some embodiments, the effective amount of an antibody and the effective amount of the antineoplastic agent are a combined synergistic amount. In some embodiments, the subject has a tumor or a cancer, is at risk of developing the tumor or the cancer, or is suspected of having the tumor or the cancer. In some embodiments, the cancer is selected from colorectal cancer. In some embodiments, the antibody is administered to the subject at a concentration of 0.200 mg/mL to 2.00 mg/mL, 0.500 mg/mL to 1.50 mg/mL, 0.700 mg/mL to 0.875 mg/mL, or 0.875 mg/mL to 1.25 mg/mL. In some embodiments, the antibody is administered to the subject at a concentration of 1.25 mg/mL to 2.50 mg/mL, 2.50 mg/mL to 5.00 mg/mL, 5.00 mg/mL to 10.0 mg/mL, 10.0 mg/mL to 25.0 mg/mL, 25.0 mg/mL to 50.0 mg/mL, 50.0 mg/mL to 100 mg/mL, 100 mg/mL to 150 mg/mL, or 150 mg/mL to 200 mg/mL.

Also disclosed herein are uses of any antibody described herein in the manufacture of a medicament for the treatment of a tumor or cancer.

### BRIEF DESCRIPTION OF THE DRAWINGS

FIG. 1 presents illustrative data showing the effects on cancer cell viability of various antibody constructs bound to DX8951-conjugated secondary antibodies, in accordance with embodiments.

FIG. 2A presents illustrative data showing affinity binding of chimeric AT-19 family antibodies and AT-19 E10 antibody to human FLT-3-expressing HEK293 cells, in accordance with embodiments. FIG. 2B presents illustrative data showing affinity binding of AT-19 family antibodies and AT-19 E10 antibody to cynomolgus FLT-3 protein, in accordance with embodiments.

FIG. 3A presents illustrative data showing the effects on monocyte viability of AT-19 and AT-19 E10 antibodies bound to DX8951-conjugated secondary antibodies, in accordance with embodiments. FIG. 3B presents illustrative data showing the effects on monocyte viability of AT-19 and AT-19 E10 antibodies bound to MMAE-conjugated secondary antibodies, in accordance with embodiments.

FIG. 4A presents illustrative data showing affinity binding of humanized and chimeric AT-19.20 antibodies and AT-19 E10 antibody to FLT-3 protein on MM6 cell lines, in accordance with embodiments. FIG. 4B presents illustrative data showing the effects on MM6 monocyte viability of AT-19.20 chimeric (M20c) anti-FLT-3 antibody bound to a DX8951-coupled secondary antibody, humanized AT-19.20 (M20, e.g., “m20 h”) anti-FLT3 antibody bound to DX8951-coupled secondary antibody, chimeric 16F7 anti-FLT3 antibody bound to DX8951-coupled secondary antibody, AT-19 E10 anti-FLT3 antibody bound to a DX8951-coupled secondary antibody, in accordance with embodiments.

FIG. 5A presents illustrative data showing affinity binding of humanized, chimeric, and antibody drug conjugate (M20ADC) AT-19.20 antibodies and AT-19 E10 antibody to MM6 human monocytic cells, in accordance with embodiments. FIG. 5B presents illustrative data showing binding of humanized AT19.M20 and AT19.E10 antibodies to MOLM-13 human acute myeloid leukemia cells and MV-4-11 human myelogenous leukemia cells.

FIGS. 5C and 5D present illustrative data showing the effects on acute myeloid leukemia (AML) cell viability of chimeric AT-19 and humanized AT-19 antibodies bound to drug payloads, in accordance with embodiments.

FIG. 6A presents illustrative data showing effects on MV4-11 AML cell viability of humanized AT-19 anti-FLT3 antibody bound to DX8951-coupled secondary antibody and AT-19 E10 anti-FLT3 antibody bound to DX8951-coupled secondary antibody, in accordance with embodiments. FIG. 6B presents illustrative data showing effects on MV4-11 AML cell viability of humanized AT-19 anti-FLT3 antibody directly conjugated to DXd via a GGFG linker (M20-ADC), chimeric AT-19 anti-FLT3 antibody bound to a DX8951-conjugated secondary antibody (M20c+Payload A), humanized AT-19 anti-FLT3 antibody bound to a DX8951-conjugated secondary antibody (M20 (e.g., “m20hv2”)+Payload A), and AT-19 E10 anti-FLT3 antibody bound to a DX8951-conjugated secondary antibody (AT-19 E10+Payload A), in accordance with embodiments.

FIG. 7 presents illustrative data showing affinity binding of humanized, chimeric and antibody drug conjugate (M20ADC) AT-19.20 antibodies and AT-19 E10 antibody to SEM acute lymphoblastic leukemia (ALL) cells, in accordance with embodiments.

FIGS. 8A and 8B present illustrative data showing the effects on acute lymphoblastic leukemia (ALL) cell viability of chimeric AT-19 and humanized AT-19 antibodies bound to drug-conjugated secondary antibodies, in accordance with embodiments.

FIG. 9 presents illustrative data showing effects on acute myelogenous leukemia (AML; MV4-11) cell viability of humanized AT-19.M20 anti-FLT3 antibody directly conjugated to MMAE and AT-19. E10 anti-FLT3 antibody directly conjugated to MMAE, in accordance with embodiments.

FIG. 10 presents illustrative data showing effects on acute myelogenous leukemia (AML; MOLM-13) cell viability of humanized AT-19.M20 anti-FLT3 antibody directly conjugated to MMAE and AT-19. E10 anti-FLT3 antibody, in accordance with embodiments.

FIG. 11 presents illustrative data showing effects on acute myelogenous leukemia (AML; MOLM-13) cell viability of humanized AT-19.M20 anti-FLT3 antibody directly conjugated to DXd and AT-19. E10 anti-FLT3 antibody, in accordance with embodiments.

FIG. 12 presents illustrative data showing effects on acute lymphoblastic leukemia (ALL; SEM) cell viability of humanized AT-19.M20 anti-FLT3 antibody directly conjugated to MMAE and AT-19. E10 anti-FLT3 antibody, in accordance with embodiments.

FIG. 13 presents illustrative data showing effects on acute lymphoblastic leukemia (ALL; SEM) cell viability of humanized AT-19.M20 anti-FLT3 antibody directly conjugated to DXd and AT-19. E10 anti-FLT3 antibody, in accordance with embodiments.

FIG. 14 presents illustrative data showing effects on acute myelogenous leukemia (AML; MV-4-11) cell viability of humanized AT-19.M20 anti-FLT3 antibody directly conjugated to DXd and AT-19. E10 anti-FLT3 antibody directly conjugated to DXd, in accordance with embodiments.

### DETAILED DESCRIPTION

Activating mutations of FLT3 are evident in 30% of AML patients where it can sustain the cancer by conferring growth and survival advantage. FLT3 can be an excellent target for the development of monoclonal antibody therapeutic.

The pivotal functional domain of FLT3, which can be essential for inhibiting its binding with FLT3L (FMS-related tyrosine kinase 3 ligand) and subsequently promoting the suppression of pro-cancer signaling can be targeted by antibody constructs described herein (e.g., AT19 asset family antibodies). The AT19 antibody constructs described herein were selected for their high-affinity cell surface binding capabilities and were subjected to a series of rigorous functional assays

In the present invention several anti-FLT3 monoclonal antibodies are presented. AT19 asset family members include antibody constructs (e.g., monoclonal antibody constructs) or nucleic acids encoding all or a portion thereof, comprising one or more amino acid or nucleotide sequences disclosed herein (e.g., selected from Table 2, which includes anti-FLT3 antibody construct clones AT-19.10, AT-19.20, AT-19.20 h.v1, AT-19.20 h.v2, AT-19.20 h.v3, AT-19.20 h.v4, AT-19.21, AT-19.38, AT-19.42, AT-19.80, AT-19.84, AT-19.88, B1, B32, 16F7, 7F10, and ALT-anti-FLT3 (also referred to herein as AT-19 E10 or E10)).

Monoclonal antibodies can exert anti-tumor effects through multiple mechanisms. Binding of the monoclonal antibody to its target antigen can block interaction with endogenous partner biomolecules and inhibit functionality. Additionally, the antibody therapeutic can recruit immune cells via effector function of the FC domain. This can then lead to the removal of the cancer cell through various mechanisms including antibody-dependent cellular cytotoxicity (ADCC), antibody-dependent cellular phagocytosis (ADCP), and complement-dependent cytotoxicity (CDC).

The AT19 assets described herein can be used to elicit antibody-dependent cellular cytotoxicity (ADCC). In this mechanism the FC region of the anti-FLT3 antibody recruits immune effector cells to kill the target cell. Furthermore, the monoclonal antibodies disclosed herein can be purposed in an antibody drug conjugate (ADC) modality. In this instance the antibody is conjugated to a cytotoxic agent which is then delivered to the cancer cell. ADCs created using anti-FLT3 monoclonal antibodies described herein offer a highly specific mechanism for targeting AML cells. In some cases, ADCs created using anti-FLT3 monoclonal antibodies described herein offer a highly specific mechanism for targeting acute lymphoblastic leukemia (ALL) cells. In some cases, an anti-FLT3 antibody described herein can comprise (e.g., be conjugated to or non-covalently associated with) a drug payload such as monomethyl auristatin E (MMAE), DXd, DX8951 (DX-8951), pyrrolobenzodiazepine dimer, and/or a calicheamicin, such as N-acetyl-gamma-calicheamicin. For instance, an anti-FLT3 antibody described herein and formulated as an ADC can comprise a drug payload, e.g., wherein the drug payload is selected from monomethyl auristatin E (MMAE), DXd, DX8951 (DX-8951), pyrrolobenzodiazepine dimer, and/or a calicheamicin, such as N-acetyl-gamma-calicheamicin. In some cases, an anti-FLT3 antibody described herein (e.g., an AT-19 family antibody comprising one or more sequences shown in Table 2, or AT-19 E10, which is also referred to herein as ALT-Anti-Flt3) can be conjugated directly to a drug payload (e.g., MMAE, or a topoisomerase inhibitor, such as DXd or DX5981) by a linker (e.g., a tetrapeptide linker, such as GGFG, or a different linker, such as MC-Val-Cit-PAB). In some cases, an anti-FLT3 antibody described herein (e.g., an AT-19 family antibody comprising one or more sequences shown in Table 2, or AT-19 E10) can be indirectly bound to a drug payload (e.g., MMAE, or a topoisomerase inhibitor, such as DXd or DX8951) by a secondary antibody that specifically binds to the anti-FLT3 primary antibody. For example, an antibody-drug conjugate (ADC) described herein can comprise an anti-FLT3 AT-19 family antibody (e.g., AT-19.10, AT-19.20, AT-19.20 h.v1, AT-19.20 h.v2, AT-19.20 h.v3, AT-19.20 h.v4, AT-19.21, AT-19.38, AT-19.42, AT-19.80, AT-19.84, AT-19.88, B1, B32, 16F7, 7F10) conjugated directly to DXd via a GGFG linker molecule. In some embodiments, an antibody-drug conjugate (ADC) described herein can comprise an anti-FLT3 AT-19 family antibody (e.g., AT-19.10, AT-19.20, AT-19.20 h.v1, AT-19.20 h.v2, AT-19.20 h.v3, AT-19.20 h.v4, AT-19.21, AT-19.38, AT-19.42, AT-19.80, AT-19.84, AT-19.88, B1, B32, 16F7, 7F10) conjugated directly to MMAE via a GGFG linker molecule. In some embodiments, an antibody-drug conjugate (ADC) described herein can comprise an anti-FLT3 AT-19 family antibody (e.g., AT-19.10, AT-19.20, AT-19.20 h.v1, AT-19.20 h.v2, AT-19.20 h.v3, AT-19.20 h.v4, AT-19.21, AT-19.38, AT-19.42, AT-19.80, AT-19.84, AT-19.88, B1, B32, 16F7, 7F10) conjugated directly to DX8951 via a GGFG linker molecule. In some embodiments, an antibody-drug conjugate (ADC) described herein can comprise an anti-FLT3 AT-19 family antibody (e.g., AT-19.10, AT-19.20, AT-19.20 h.v1, AT-19.20 h.v2, AT-19.20 h.v3, AT-19.20 h.v4, AT-19.21, AT-19.38, AT-19.42, AT-19.80, AT-19.84, AT-19.88, B1, B32, 16F7, 7F10) conjugated directly to MMAE via a MC-Val-Cit-PAB linker molecule. In some embodiments, an antibody-drug conjugate (ADC) described herein can comprise an anti-FLT3 AT-19 family antibody (e.g., AT-19.10, AT-19.20, AT-19.20 h.v1, AT-19.20 h.v2, AT-19.20 h.v3, AT-19.20 h.v4, AT-19.21, AT-19.38, AT-19.42, AT-19.80, AT-19.84, AT-19.88, B1, B32, 16F7, 7F10) conjugated directly to DXd via a MC-Val-Cit-PAB linker molecule. In some embodiments, an antibody-drug conjugate (ADC) described herein can comprise an anti-FLT3 AT-19 family antibody (e.g., AT-19.10, AT-19.20, AT-19.20 h.v1, AT-19.20 h.v2, AT-19.20 h.v3, AT-19.20 h.v4, AT-19.21, AT-19.38, AT-19.42, AT-19.80, AT-19.84, AT-19.88, B1, B32, 16F7, 7F10) conjugated directly to DX8951 via a MC-Val-Cit-PAB linker molecule. In some implementations, an anti-FLT3 antibody described herein (e.g., for use in treating a patient suspected of having, at increased risk of having, or having a tumor or cancer such as acute myeloid leukemia (AML) or acute lymphoblastic leukemia (ALL)) can comprise an AT-19 E10 antibody (for instance an antibody having a heavy chain variable region according to SEQ ID NO: 194 and a light chain variable region according to SEQ ID NO: 193) and a drug payload (e.g., a topoisomerase inhibitor or a tubulin inhibitor such as an auristatin). In some embodiments, an antibody-drug conjugate (ADC) described herein can comprise an anti-FLT3 AT-19 E10 antibody (e.g., ALT-Anti-Flt3) conjugated directly to MMAE, DXd, or DX8951 via a linker molecule. In some cases, an AT-19 E10 antibody can comprise a heavy chain variable region having an amino acid sequence as shown in SEQ ID NO: 194 and a light chain variable region having an amino acid sequence as shown in SEQ ID NO:193. In some cases, a nucleotide sequence encoding an AT-19 E10 antibody can comprise a nucleotide sequence according to SEQ ID NO:195 and a nucleotide sequence according to SEQ ID NO: 196. In some embodiments, an antibody-drug conjugate (ADC) described herein can comprise an anti-FLT3 AT-19 E10 antibody (e.g., ALT-Anti-Flt3) conjugated directly to DXd via a GGFG linker molecule. In some embodiments, an antibody-drug conjugate (ADC) described herein can comprise an anti-FLT3 AT-19 E10 antibody (e.g., ALT-Anti-Flt3) conjugated directly to DXd via a MC-Val-Cit-PAB linker molecule. In some embodiments, an antibody-drug conjugate (ADC) described herein can comprise an anti-FLT3 AT-19 E10 antibody (e.g., ALT-Anti-Flt3) conjugated directly to MMAE via a GGFG linker molecule. In some embodiments, an antibody-drug conjugate (ADC) described herein can comprise an anti-FLT3 AT-19 E10 antibody (e.g., ALT-Anti-Flt3) conjugated directly to MMAE via a MC-Val-Cit-PAB linker molecule.

The AT19 assets described herein can be used to elicit antibody-dependent cellular phagocytosis (ADCP). In this mechanism the FC region of the anti-FLT3 antibody can recruit immune effector cells called macrophages to eliminate the target cell by phagocytosis.

The AT19 assets described herein can be used to elicit complement-dependent cytotoxicity (CDC). In this mechanism the FC region of the anti-FLT3 antibody can recruit complement proteins to kill the target cell.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof, or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples of nucleosides include cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may, in embodiments, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be considered when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., FLT3) in other proteins with different numbering systems. For example, by performing a simple sequence alignment with a protein (e.g., FLT3) the identity and location of residues corresponding to specific positions of the protein are identified in other protein sequences aligning to the protein. For example, a selected residue in a selected antibody (or Fab domain) corresponds to light chain threonine at Kabat position 40, when the selected residue occupies the same essential spatial or other structural relationship as a light chain threonine at Kabat position 40. In some embodiments, where a selected protein is aligned for maximum homology with the light chain of an antibody (or Fab domain), the position in the aligned selected protein aligning with threonine 40 is said to correspond to threonine 40. Instead of a primary sequence alignment, a three dimensional structural alignment can also be used, e.g., where the structure of the selected protein is aligned for maximum correspondence with the light chain threonine at Kabat position 40, and the overall structures compared. In this case, an amino acid that occupies the same essential position as threonine 40 in the structural model is said to correspond to the threonine 40 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region, involved in binding the target antigen, and a constant (“C”) region that interacts with other components of the immune system. The light and heavy chain variable regions (also referred to herein as light chain variable (VL) domain and heavy chain variable (VH) domain, respectively) come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab₂, bispecific Fab₂, trispecific Fab₃, monovalent IgGs, scFv, bispecific antibodies, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1”, “CDR 112” and “CDR H3” as provided herein refer to the complementarity determining regions (CDR) 1, 2, and 3 of the variable heavy (H) chain of an antibody. In embodiments, the variable heavy chain provided herein includes in N-terminal to C-terminal direction a CDR H1, a CDR H2 and a CDR H3. In embodiments, the CDRs of the light chain are referred to as CDR1, CDR2, and CDR3 of VL and the CDRs of the heavy chain are referred to as CDR1, CDR2, and CDR3 of VH. See, for example the tables as provided herein.

The terms “FR L1”, “FR L2”, “FR L3” and “FR L4” as provided herein are used according to their common meaning in the art and refer to the framework regions (FR) 1, 2, 3 and 4 of the variable light (L) chain of an antibody. In embodiments, the variable light chain provided herein includes in N-terminal to C-terminal direction a FR L1, a FR L2, a FR L3 and a FR L4. Likewise, the terms “FR H1”, “FR 112”, “FR H3” and “FR 114” as provided herein are used according to their common meaning in the art and refer to the framework regions (FR) 1, 2, 3 and 4 of the variable heavy (H) chain of an antibody. In embodiments, the variable heavy chain provided herein includes in N-terminal to C-terminal direction a FR H1, a FR H2, a FR H3 and a FR H4.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL), variable light chain (VL) domain or light chain variable region and variable heavy chain (VH), variable heavy chain (VH) domain or heavy chain variable region refer to these light and heavy chain regions, respectively. The terms variable light chain (VL), variable light chain (VL) domain and light chain variable region as referred to herein may be used interchangeably. The terms variable heavy chain (VH), variable heavy chain (VH) domain and heavy chain variable region as referred to herein may be used interchangeably. The Fc (i.e. fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins. The term “light chain” is used according to its ordinary meaning in the biological arts, and refers to the polypeptide formed by a light chain variable domain (VL) and a light chain constant domain (CL). Likewise, the term “heavy chain” is used according to its ordinary meaning in the biological arts, and refers to the polypeptide formed by a heavy chain variable domain (VH) and one or more heavy chain constant domains (CH1, CH2, CH3).

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)). The term “antibody” as referred to herein further includes antibody variants such as single domain antibodies. Thus, in embodiments an antibody includes a single monomeric variable antibody domain. Thus, in embodiments, the antibody, includes a variable light chain (VL) domain or a variable heavy chain (VH) domain. In embodiments, the antibody is a variable light chain (VL) domain or a variable heavy chain (VH) domain. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies (mAb) refer to antibodies derived from a single clone. Techniques to produce single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A single-chain variable fragment (scFv) is typically a fusion protein of the variable domains of the heavy (VH) and light chain (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (e.g, variable region including domain VH and VL) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

Techniques for conjugating therapeutic agents to antibodies are well known (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)). As used herein, the term “antibody-drug conjugate” or “ADC” refers to a therapeutic agent conjugated or otherwise covalently bound to an antibody.

A “therapeutic agent” as referred to herein, is a composition useful in treating or preventing a disease such as cancer (e.g., leukemia). In embodiments, the therapeutic agent is an anti-cancer agent. “Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “ligand” refers to an agent, e.g., a polypeptide or other molecule, capable of binding to a receptor or antibody, antibody variant, antibody region or fragment thereof.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “FLT3” or “fms-like tyrosine kinase 3” as used herein refers to any recombinant or naturally-occurring forms of fms-like tyrosine kinase 3 Activation (FLT3) or variants or homologs thereof that maintain FLT3 activity (e.g. within at least 50%, 80%, 90%, 95%, 96%, 97%, 98%, 99% or 100% activity compared to FLT3). In embodiments, the variants or homologs have at least 90%, 95%, 96%, 97%, 98%, 99% or 100% amino acid sequence identity across the whole sequence or a portion of the sequence (e.g. a 10, 20, 50, 100, 150 or 200 continuous amino acid portion) compared to a naturally occurring FLT3 polypeptide. In embodiments, FLT3 is substantially identical to the protein identified by the UniProt reference number P36888 or a variant or homolog having substantial identity thereto. In embodiments, FLT3 is the protein identified by the UniProt reference number P36888 or a variant or homolog having substantial identity thereto.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetofection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

When the label or detectable moiety is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups. The chelate is normally linked to the PSMA antibody or functional antibody fragment by a group, which enables the formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antibodies and carriers described herein. Macrocyclic chelates such as NOTA, DOTA, and TETA are of use with a variety of metals and radiometals including, but not limited to, radionuclides of gallium, yttrium and copper, respectively. Other ring-type chelates such as macrocyclic polyethers, which are of interest for stably binding nuclides, such as ²²³Ra for RAIT may be used. In certain embodiments, chelating moieties may be used to attach a PET imaging agent, such as an Al—¹⁸F complex, to a targeting molecule for use in PET analysis.

“Contacting” is used in accordance with its plain ordinary meaning and refers to the process of allowing at least two distinct species (e.g. antibodies and antigens) to become sufficiently proximal to react, interact, or physically touch. It should be appreciated; however, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a pharmaceutical composition as provided herein and a cell. In embodiments contacting includes, for example, allowing a pharmaceutical composition as described herein to interact with a cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

A “stem cell” as provided herein refers to a cell characterized by the ability of self-renewal through mitotic cell division and the potential to differentiate into a tissue or an organ. Among mammalian stem cells, embryonic stem cells (ES cells) and somatic stem cells (e.g., hematopoietic stem cells (HSC)) can be distinguished. Embryonic stem cells reside in the blastocyst and give rise to embryonic tissues, whereas somatic stem cells reside in adult tissues for the purpose of tissue regeneration and repair.

As defined herein, the term “inhibition”, “inhibit”, “inhibiting” and the like in reference to cell proliferation (e.g., cancer cell proliferation) means negatively affecting (e.g., decreasing proliferation) or killing the cell. In some embodiments, inhibition refers to reduction of a disease or symptoms of disease (e.g., cancer, cancer cell proliferation). Thus, inhibition includes, at least in part, partially or totally blocking stimulation, decreasing, preventing, or delaying activation, or inactivating, desensitizing, or down-regulating signal transduction or enzymatic activity or the amount of a protein. Similarly, an “inhibitor” is a compound or protein that inhibits a receptor or another protein, e.g., by binding, partially or totally blocking, decreasing, preventing, delaying, inactivating, desensitizing, or down-regulating activity (e.g., a receptor activity or a protein activity).

As defined herein, the term “inhibition”, “inhibit”, “inhibiting” and the like in reference to a protein-inhibitor interaction means negatively affecting (e.g. decreasing) the activity or function of the protein (e.g. FLT3) relative to the activity or function of the protein in the absence of the inhibitor. In embodiments inhibition means negatively affecting (e.g. decreasing) the concentration or levels of a protein (e.g. FLT3) relative to the concentration or level of the protein in the absence of the inhibitor. In embodiments inhibition refers to reduction of a disease or symptoms of disease. In embodiments, inhibition refers to a reduction in the activity of a protein (e.g. FLT3). Thus, inhibition includes, at least in part, partially or totally blocking stimulation, decreasing, preventing, or delaying activation, or inactivating, desensitizing, or down-regulating signal transduction or enzymatic activity or the amount of a protein (e.g. FLT3). In embodiments, inhibition refers to a reduction of activity of a protein (e.g. FLT3) resulting from a direct interaction (e.g. an inhibitor binds to the protein). In embodiments, inhibition refers to a reduction of activity of a protein (e.g. FLT3) from an indirect interaction (e.g. an inhibitor binds to a protein that activates the protein, thereby preventing target protein activation).

Thus, the terms “inhibitor,” “repressor” or “antagonist” or “downregulator” interchangeably refer to a substance capable of detectably decreasing the expression or activity of a given gene or protein (e.g. FLT3). The antagonist can decrease expression or activity of a protein (e.g. FLT3) by 10%, 20%, 30%, 40%, 50%, 60%, 70%, 80%, 90% or more in comparison to a control in the absence of the antagonist. In certain instances, a protein's (e.g. FLT3) expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, synoviocytes, synovial fluid, synovial tissue, fibroblast-like synoviocytes, macrophagelike synoviocytes, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with the compounds or methods provided herein. The disease may be a cancer. In some further instances, “cancer” refers to human cancers and carcinomas, sarcomas, adenocarcinomas, lymphomas, leukemias, including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including acute myeloid leukemia (AML), acute lymphoblastic leukemia (ALL), and CML), or multiple myeloma.

As used herein, the term “cancer” refers to all types of cancer, neoplasm or malignant tumors found in mammals (e.g., humans), including leukemia, carcinomas and sarcomas. Exemplary cancers that may be treated with a compound or method provided herein include breast cancer, colon cancer, colorectal cancer, kidney cancer, leukemia, lung cancer (e.g., non-small cell lung cancer), melanoma, ovarian cancer, prostate cancer, renal cancer, pancreatic cancer, brain cancer (e.g., glioblastomas and/or astrocytomas), liver cancer, gastric cancer or a sarcoma. In some cases, “cancer” can refer to ex vivo or in vitro tumor or cancer cells, for example, in a diagnostic or research context. As shown herein, embodiments of AT-19 family antibodies can be especially useful in commercial compositions and formulated treatments for reducing the viability of acute lymphoblastic leukemia (ALL), acute myeloid leukemia (AML), colorectal cancer, gastric cancer, and/or pancreatic cancer, for instance by targeting one or more antineoplastic drug payloads (e.g., comprising DX8951, DXd, or MMAE) to the leukemia or cancer cell(s).

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute myeloid leukemia, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. In some cases, cell migration (e.g., increased cell migration relative to a control cell) can be indicative of a risk of metastasis or a tendency to metastasize in a tumor tissue or can potentiate metastasis, cancer tissue, tumor cell, or cancer cell. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g., cancer (e.g. leukemia, acute myeloid leukemia)) means that the disease (e.g., cancer (e.g. leukemia, acute myeloid leukemia)) is caused by (in whole or in part), or a symptom of the disease is caused by (in whole or in part) the substance or substance activity or function. Alternatively, the substance (e.g., FLT3) may be an indicator of the disease (e.g., cancer (e.g. leukemia, acute myeloid leukemia)). Thus, an associated substance may serve as a means of targeting disease tissue (e.g., cancer cells (e.g., leukemia stem cells, acute myeloid leukemia cells)).

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

A “therapeutic agent” as referred to herein, is a composition useful in treating or preventing a disease such as cancer (e.g., leukemia). In embodiments, the therapeutic agent is an anti-cancer agent. “Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus, in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

A “synergistic amount” as used herein refers to the sum of a first amount (e.g., an amount of a compound provided herein) and a second amount (e.g., a therapeutic agent) that results in a synergistic effect (i.e. an effect greater than an additive effect). Therefore, the terms “synergy”, “synergism”, “synergistic”, “combined synergistic amount”, and “synergistic therapeutic effect” which are used herein interchangeably, refer to a measured effect of the compound administered in combination where the measured effect is greater than the sum of the individual effects of each of the compounds provided herein administered alone as a single agent.

### Antibody Compositions

Provided herein are, inter alia, antibodies (e.g., humanized antibodies, monoclonal antibodies) and antibody compositions (e.g., scFvs, bispecific antibodies) that are capable of binding FLT3. The antibodies and antibody compositions provided herein include novel light and heavy chain domain CDRs and framework regions, and bind FLT3 with high efficiency and specificity, thereby effectively targeting these target proteins and/or cells expressing the target proteins. The light and heavy chain domains of the antibodies provided herein may form part of recombinant proteins also referred to herein as antibody compositions (e.g., IgG (for instance, IgG1), scFv, or bispecific antibodies) to be used, inter alia, as cancer therapeutics and for diagnostic purposes.

Anti-FLT3 antibody constructs disclosed herein can comprise (or be encoded by) one or more novel molecular sequences selected from among SEQ ID NO:1 through SEQ ID NO: 196. Antibodies and antibody fragments (as well as nucleic acid vectors encoding such antibodies and antibody fragments) comprising one or more of these sequences are contemplated herein. Collectively, such antibody constructs and nucleic acid vectors are referred to as the “AT19” or “AT-19” series of assets. For example, “AT-19.10” or “AT19.10” antibodies, antibody fragments, or vectors can comprise one or more molecular sequences selected from among SEQ ID NO:1 through SEQ ID NO:8. In some cases, AT-19 antibodies can be mouse anti-FLT3 antibodies. In some cases, AT-19 antibodies can be chimeric anti-FLT3 antibodies. Among the AT-19 series assets contemplated herein are AT-19.10, AT-19.20, AT-19.20 h.v1, AT-19.20 h.v2, AT-19.20 h.v3, AT-19.20 h.v4, AT-19.21, AT-19.38, AT-19.42, AT-19.80, AT-19.84, AT-19.88, B1, B32, 16F7, or 7F10 antibodies and antibody fragments (e.g., chimeric antibodies and antibody fragments), as well as nucleic acid vectors encoding such antibodies and antibody fragments. In some cases, AT-19 series assets contemplated herein can be humanized anti-FLT3 antibodies or fragments thereof.

In some cases, an anti-FLT3 antibody disclosed herein can include a light chain variable domain and a heavy chain variable domain, wherein the light chain variable domain includes: a CDR L1 as set forth in SEQ ID NO: 17, a CDR L2 as set forth in SEQ ID NO: 18 and a CDR L3 as set forth in SEQ ID NO: 19; and wherein the heavy chain variable domain includes: a CDR H1 as set forth in SEQ ID NO: 20, a CDR H2 as set forth in SEQ ID NO:21, and a CDR H3 as set forth in SEQ ID NO:22. In some cases, an anti-FLT3 antibody can include a light chain variable domain having an amino acid sequence with at least 80%, 85%, 90%, 95%, 98%, 99%, or 100% sequence identity with the sequence of SEQ ID NO:7. In some cases, an anti-FLT3 antibody can include a heavy chain variable domain having an amino acid sequence with at least 80%, 85%, 90%, 95%, 98%, 99%, or 100% sequence identity with the sequence of SEQ ID NO:8. In some cases, an anti-FLT3 antibody can include a light chain variable domain having an amino acid sequence with at least 80%, 85%, 90%, 95%, 98%, 99%, or 100% sequence identity with the sequence of SEQ ID NO:15. In some cases, an anti-FLT3 antibody can include a heavy chain variable domain having an amino acid sequence with at least 80%, 85%, 90%, 95%, 98%, 99%, or 100% sequence identity with the sequence of SEQ ID NO: 16. In some cases, an anti-FLT3 antibody can include a light chain variable domain having an amino acid sequence with at least 80%, 85%, 90%, 95%, 98%, 99%, or 100% sequence identity with the sequence of SEQ ID NO:23. In some cases, an anti-FLT3 antibody can include a heavy chain variable domain having an amino acid sequence with at least 80%, 85%, 90%, 95%, 98%, 99%, or 100% sequence identity with the sequence of SEQ ID NO:24. In some cases, an anti-FLT3 antibody can include a light chain variable domain having an amino acid sequence with at least 80%, 85%, 90%, 95%, 98%, 99%, or 100% sequence identity with the sequence of SEQ ID NO:31. In some cases, an anti-FLT3 antibody can include a heavy chain variable domain having an amino acid sequence with at least 80%, 85%, 90%, 95%, 98%, 99%, or 100% sequence identity with the sequence of SEQ ID NO:32. In some cases, an anti-FLT3 antibody can include a light chain variable domain having an amino acid sequence with at least 80%, 85%, 90%, 95%, 98%, 99%, or 100% sequence identity with the sequence of SEQ ID NO:39. In some cases, an anti-FLT3 antibody can include a heavy chain variable domain having an amino acid sequence with at least 80%, 85%, 90%, 95%, 98%, 99%, or 100% sequence identity with the sequence of SEQ ID NO:40. In some cases, an anti-FLT3 antibody can include a light chain variable domain having an amino acid sequence with at least 80%, 85%, 90%, 95%, 98%, 99%, or 100% sequence identity with the sequence of SEQ ID NO:47. In some cases, an anti-FLT3 antibody can include a heavy chain variable domain having an amino acid sequence with at least 80%, 85%, 90%, 95%, 98%, 99%, or 100% sequence identity with the sequence of SEQ ID NO:48. In some cases, an anti-FLT3 antibody can include a light chain variable domain having an amino acid sequence with at least 80%, 85%, 90%, 95%, 98%, 99%, or 100% sequence identity with the sequence of SEQ ID NO:55. In some cases, an anti-FLT3 antibody can include a heavy chain variable domain having an amino acid sequence with at least 80%, 85%, 90%, 95%, 98%, 99%, or 100% sequence identity with the sequence of SEQ ID NO:56. In some cases, an anti-FLT3 antibody can include a light chain variable domain having an amino acid sequence with at least 80%, 85%, 90%, 95%, 98%, 99%, or 100% sequence identity with the sequence of SEQ ID NO:63. In some cases, an anti-FLT3 antibody can include a heavy chain variable domain having an amino acid sequence with at least 80%, 85%, 90%, 95%, 98%, 99%, or 100% sequence identity with the sequence of SEQ ID NO:64. In some cases, an anti-FLT3 antibody can include a light chain variable domain having an amino acid sequence with at least 80%, 85%, 90%, 95%, 98%, 99%, or 100% sequence identity with the sequence of SEQ ID NO:71. In some cases, an anti-FLT3 antibody can include a heavy chain variable domain having an amino acid sequence with at least 80%, 85%, 90%, 95%, 98%, 99%, or 100% sequence identity with the sequence of SEQ ID NO:72. In some cases, an anti-FLT3 antibody can include a light chain variable domain having an amino acid sequence with at least 80%, 85%, 90%, 95%, 98%, 99%, or 100% sequence identity with the sequence of SEQ ID NO:79. In some cases, an anti-FLT3 antibody can include a heavy chain variable domain having an amino acid sequence with at least 80%, 85%, 90%, 95%, 98%, 99%, or 100% sequence identity with the sequence of SEQ ID NO:80. In some cases, an anti-FLT3 antibody can include a light chain variable domain having an amino acid sequence with at least 80%, 85%, 90%, 95%, 98%, 99%, or 100% sequence identity with the sequence of SEQ ID NO:87. In some cases, an anti-FLT3 antibody can include a heavy chain variable domain having an amino acid sequence with at least 80%, 85%, 90%, 95%, 98%, 99%, or 100% sequence identity with the sequence of SEQ ID NO:88. In some cases, an anti-FLT3 antibody can include a light chain variable domain having an amino acid sequence with at least 80%, 85%, 90%, 95%, 98%, 99%, or 100% sequence identity with the sequence of SEQ ID NO:95. In some cases, an anti-FLT3 antibody can include a heavy chain variable domain having an amino acid sequence with at least 80%, 85%, 90%, 95%, 98%, 99%, or 100% sequence identity with the sequence of SEQ ID NO:96. In some cases, an anti-FLT3 antibody can include a light chain variable domain having an amino acid sequence with at least 80%, 85%, 90%, 95%, 98%, 99%, or 100% sequence identity with the sequence of SEQ ID NO: 103. In some cases, an anti-FLT3 antibody can include a heavy chain variable domain having an amino acid sequence with at least 80%, 85%, 90%, 95%, 98%, 99%, or 100% sequence identity with the sequence of SEQ ID NO:104. In some cases, an anti-FLT3 antibody can include a light chain variable domain having an amino acid sequence with at least 80%, 85%, 90%, 95%, 98%, 99%, or 100% sequence identity with the sequence of SEQ ID NO:111. In some cases, an anti-FLT3 antibody can include a heavy chain variable domain having an amino acid sequence with at least 80%, 85%, 90%, 95%, 98%, 99%, or 100% sequence identity with the sequence of SEQ ID NO: 112. In some cases, an anti-FLT3 antibody can include a light chain variable domain having an amino acid sequence with at least 80%, 85%, 90%, 95%, 98%, 99%, or 100% sequence identity with the sequence of SEQ ID NO:119. In some cases, an anti-FLT3 antibody can include a heavy chain variable domain having an amino acid sequence with at least 80%, 85%, 90%, 95%, 98%, 99%, or 100% sequence identity with the sequence of SEQ ID NO: 120. In some cases, an anti-FLT3 antibody can include a light chain variable domain having an amino acid sequence with at least 80%, 85%, 90%, 95%, 98%, 99%, or 100% sequence identity with the sequence of SEQ ID NO:127. In some cases, an anti-FLT3 antibody can include a heavy chain variable domain having an amino acid sequence with at least 80%, 85%, 90%, 95%, 98%, 99%, or 100% sequence identity with the sequence of SEQ ID NO:128. In some cases, an anti-FLT3 antibody can include a light chain variable domain having an amino acid sequence with at least 80%, 85%, 90%, 95%, 98%, 99%, or 100% sequence identity with the sequence of SEQ ID NO: 193. In some cases, an anti-FLT3 antibody can include a heavy chain variable domain having an amino acid sequence with at least 80%, 85%, 90%, 95%, 98%, 99%, or 100% sequence identity with the sequence of SEQ ID NO:194. In some cases, association (e.g., binding) of an anti-FLT3 antibody described herein with at least a portion of a FLT3 protein can inhibit the adhesion, proliferation, survival, migration, spread (e.g., metastasis), or growth (e.g., diameter) of one or more types of tumor cells, tumor tissues, cancer cells, or cancer tissues (e.g., breast cancer, pancreatic cancer, ovarian cancer, renal cancer, colorectal cancer, brain cancer such as a glioblastoma or astrocytoma, or lung cancer such as a non-small cell lung cancer) in an in vitro or in vivo context. In some cases, an anti-FLT3 antibody disclosed herein can be effective at inhibiting one or more of adhesion, proliferation, survival, migration, spread (e.g., metastasis), or growth (e.g., diameter) of one or more tumor cells, tumor tissues, cancer cells, or cancer tissues (e.g., wherein the one or more cells or tissues comprises two or more tumor or cancer types, for example, selected from breast cancer, pancreatic cancer, ovarian cancer, renal cancer, colorectal cancer, brain cancer such as a glioblastoma or astrocytoma, or lung cancer such as a non-small cell lung cancer). In some cases, an anti-FLT3 antibody disclosed herein can be effective at increasing or maintaining viability of peripheral mononuclear blood cell(s), e.g., in the context of at least one (e.g., one, two, three, four, or more than four) in vivo or in vitro tumor or cancer cell/tissue types described herein.

As described herein, a “light chain variable (VL) domain” as provided herein refers to the variable region of the light chain of an antibody, an antibody variant or fragment thereof. Likewise, the “heavy chain variable (VH) domain” as provided herein refers to the variable region of the heavy chain of an antibody, an antibody variant or fragment thereof. The light chain variable domain and the heavy chain variable domain together form the paratope, which binds an antigen (epitope). The paratope or antigen-binding site is formed at the N-terminus of an antibody, an antibody variant or fragment thereof. In embodiments, the light chain variable (VL) domain includes CDR L1, CDR L2, CDR L3 and FR L1, FR L2, FR L3 and FR L4 (framework regions) of an antibody light chain. In embodiments, the heavy chain variable (VH) domain includes CDR H1, CDR H2, CDR H3 and FR H1, FR H2, FR H3 and FR H4 (framework regions) of an antibody heavy chain. In embodiments, the light chain variable (VL) domain and a light chain constant (CL) domain form part of an antibody light chain. In embodiments, the heavy chain variable (VH) domain and a heavy chain constant (CH1) domain form part of an antibody heavy chain. In embodiments, the heavy chain variable (VH) domain and one or more heavy chain constant (CH1, CH2, or CH3) domains form part of an antibody heavy chain. Thus, in embodiments, the light chain variable (VL) domain forms part of an antibody. In embodiments, the heavy chain variable (VH) domain forms part of an antibody. In embodiments, the light chain variable (VL) domain forms part of a therapeutic antibody. In embodiments, the heavy chain variable (VH) domain forms part of a therapeutic antibody. In embodiments, the light chain variable (VL) domain forms part of a human antibody. In embodiments, the heavy chain variable (VH) domain forms part of a human antibody. In embodiments, the light chain variable (VL) domain forms part of a humanized antibody. In embodiments, the heavy chain variable (VH) domain forms part of a humanized antibody. In embodiments, the light chain variable (VL) domain forms part of a chimeric antibody. In embodiments, the heavy chain variable (VH) domain forms part of a chimeric antibody. In embodiments, the light chain variable (VL) domain forms part of an antibody fragment. In embodiments, the heavy chain variable (VH) domain forms part of an antibody fragment. In embodiments, the light chain variable (VL) domain forms part of an antibody variant. In embodiments, the heavy chain variable (VH) domain forms part of an antibody variant. In embodiments, the light chain variable (VL) domain forms part of a Fab. In embodiments, the heavy chain variable (VH) domain forms part of a Fab. In embodiments, the light chain variable (VL) domain forms part of a scFv. In embodiments, the heavy chain variable (VH) domain forms part of a scFv.

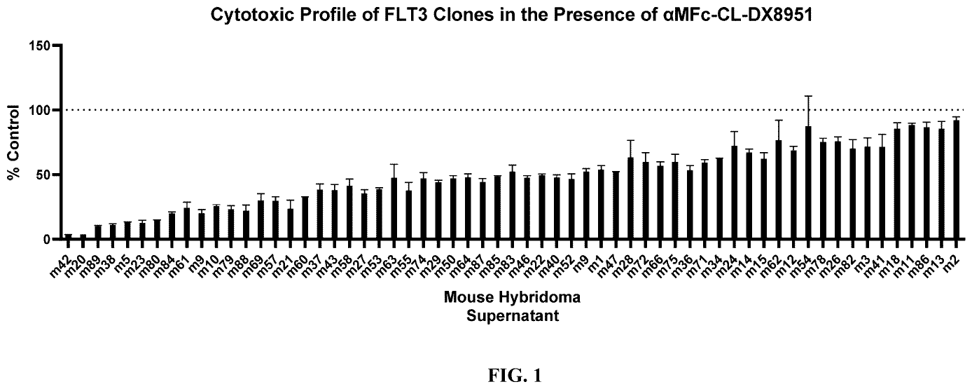

In embodiments, the antibody is a humanized antibody. In embodiments, the antibody is a chimeric antibody. In embodiments, the antibody is a full length antibody. In embodiments, the antibody is a F(ab)2 fragment. In embodiments, the antibody is a Fab′ fragment. In embodiments, the antibody is a single chain antibody (scFv). In embodiments, the light chain variable domain and the heavy chain variable domain form part of a scFv. In embodiments, the full length antibody, F(ab)2 fragment, scFv, or Fab′ fragment includes the sequence of SEQ ID NO:1. In embodiments, the full length antibody, F(ab)2 fragment, scFv, or Fab′ fragment includes the sequence of SEQ ID NO:2. In embodiments, the full length antibody, F(ab)2 fragment, scFv, or Fab′ fragment includes the sequence of SEQ ID NO:3. In embodiments, the full length antibody, F(ab)2 fragment, scFv, or Fab′ fragment includes the sequence of SEQ ID NO:4. In embodiments, the full length antibody, F(ab)2 fragment, scFv, or Fab′ fragment includes the sequence of SEQ ID NO:5. In embodiments, the full length antibody, F(ab)2 fragment, scFv, or Fab′ fragment includes the sequence of SEQ ID NO:6. In embodiments, the full length antibody, F(ab)2 fragment, scFv, or Fab′ fragment includes the sequence of SEQ ID NO:7. In embodiments, the full length antibody, F(ab)2 fragment, scFv, or Fab′ fragment includes the sequence of SEQ ID NO:8. In embodiments, the full length antibody, F(ab)2 fragment, scFv, or Fab′ fragment includes the sequence of SEQ ID NO:7 and the sequence of SEQ ID NO:8. In embodiments, the full length antibody, F(ab)2 fragment, scFv, or Fab′ fragment includes the sequence of SEQ ID NO:9. In embodiments, the full length antibody, F(ab)2 fragment, scFv, or Fab′ fragment includes the sequence of SEQ ID NO: 10. In embodiments, the full length antibody, F(ab)2 fragment, scFv, or Fab′ fragment includes the sequence of SEQ ID NO: 11. In embodiments, the full length antibody, F(ab)2 fragment, scFv, or Fab′ fragment includes the sequence of SEQ ID NO: 12. In embodiments, the full length antibody, F(ab)2 fragment, scFv, or Fab′ fragment includes the sequence of SEQ ID NO: 13. In embodiments, the full length antibody, F(ab)2 fragment, scFv, or Fab′ fragment includes the sequence of SEQ ID NO: 14. In embodiments, the full length antibody, F(ab)2 fragment, scFv, or Fab′ fragment includes the sequence of SEQ ID NO: 15. In embodiments, the full length antibody, F(ab)2 fragment, scFv, or Fab′ fragment includes the sequence of SEQ ID NO: 16. In embodiments, the full length antibody, F(ab)2 fragment, scFv, or Fab′ fragment includes the sequence of SEQ ID NO: 17 and the sequence of SEQ ID NO:16. In embodiments, the full length antibody, F(ab)2 fragment, scFv, or Fab′ fragment includes the sequence of SEQ ID NO:17. In embodiments, the full length antibody, F(ab)2 fragment, scFv, or Fab′ fragment includes the sequence of SEQ ID NO: 18. In embodiments, the full length antibody, F(ab)2 fragment, scFv, or Fab′ fragment includes the sequence of SEQ ID NO: 19. In embodiments, the full length antibody, F(ab)2 fragment, scFv, or Fab′ fragment includes the sequence of SEQ ID NO:20. In embodiments, the full length antibody, F(ab)2 fragment, scFv, or Fab′ fragment includes the sequence of SEQ ID NO:21. In embodiments, the full length antibody, F(ab)2 fragment, scFv, or Fab′ fragment includes the sequence of SEQ ID NO:22. In embodiments, the full length antibody, F(ab)2 fragment, scFv, or Fab′ fragment includes the sequence of SEQ ID NO:23. In embodiments, the full length antibody, F(ab)2 fragment, scFv, or Fab′ fragment includes the sequence of SEQ ID NO:24. In embodiments, the full length antibody, F(ab)2 fragment, scFv, or Fab′ fragment includes the sequence of SEQ ID NO:23 and the sequence of SEQ ID NO:24. In embodiments, the full length antibody, F(ab)2 fragment, scFv, or Fab′ fragment includes the sequence of SEQ ID NO:25. In embodiments, the full length antibody, F(ab)2 fragment, scFv, or Fab′ fragment includes the sequence of SEQ ID NO:26. In embodiments, the full length antibody, F(ab)2 fragment, scFv, or Fab′ fragment includes the sequence of SEQ ID NO:27. In embodiments, the full length antibody, F(ab)2 fragment, scFv, or Fab′ fragment includes the sequence of SEQ ID NO:28. In embodiments, the full length antibody, F(ab)2 fragment, scFv, or Fab′ fragment includes the sequence of SEQ ID NO:29. In embodiments, the full length antibody, F(ab)2 fragment, scFv, or Fab′ fragment includes the sequence of SEQ ID NO:30. In embodiments, the full length antibody, F(ab)2 fragment, scFv, or Fab′ fragment includes the sequence of SEQ ID NO:31. In embodiments, the full length antibody, F(ab)2 fragment, scFv, or Fab′ fragment includes the sequence of SEQ ID NO:32. In embodiments, the full length antibody, F(ab)2 fragment, scFv, or Fab′ fragment includes the sequence of SEQ ID NO:31 and the sequence of SEQ ID NO:32. In embodiments, the full length antibody, F(ab)2 fragment, scFv, or Fab′ fragment includes the sequence of SEQ ID NO:33. In embodiments, the full length antibody, F(ab)2 fragment, scFv, or Fab′ fragment includes the sequence of SEQ ID NO:34. In embodiments, the full length antibody, F(ab)2 fragment, scFv, or Fab′ fragment includes the sequence of SEQ ID NO:35. In embodiments, the full length antibody, F(ab)2 fragment, scFv, or Fab′ fragment includes the sequence of SEQ ID NO:36. In embodiments, the full length antibody, F(ab)2 fragment, scFv, or Fab′ fragment includes the sequence of SEQ ID NO:37. In embodiments, the full length antibody, F(ab)2 fragment, scFv, or Fab′ fragment includes the sequence of SEQ ID NO:38. In embodiments, the full length antibody, F(ab)2 fragment, scFv, or Fab′ fragment includes the sequence of SEQ ID NO:39. In embodiments, the full length antibody, F(ab)2 fragment, scFv, or Fab′ fragment includes the sequence of SEQ ID NO:40. In embodiments, the full length antibody, F(ab)2 fragment, scFv, or Fab′ fragment includes the sequence of SEQ ID NO:39 and the sequence of SEQ ID NO:40. In embodiments, the full length antibody, F(ab)2 fragment, scFv, or Fab′ fragment includes the sequence of SEQ ID NO:41. In embodiments, the full length antibody, F(ab)2 fragment, scFv, or Fab′ fragment includes the sequence of SEQ ID NO:42. In embodiments, the full length antibody, F(ab)2 fragment, scFv, or Fab′ fragment includes the sequence of SEQ ID NO:43. In embodiments, the full length antibody, F(ab)2 fragment, scFv, or Fab′ fragment includes the sequence of SEQ ID NO:44. In embodiments, the full length antibody, F(ab)2 fragment, scFv, or Fab′ fragment includes the sequence of SEQ ID NO:45. In embodiments, the full length antibody, F(ab)2 fragment, scFv, or Fab′ fragment includes the sequence of SEQ ID NO:46. In embodiments, the full length antibody, F(ab)2 fragment, scFv, or Fab′ fragment includes the sequence of SEQ ID NO:47. In embodiments, the full length antibody, F(ab)2 fragment, scFv, or Fab′ fragment includes the sequence of SEQ ID NO:48. In embodiments, the full length antibody, F(ab)2 fragment, scFv, or Fab′ fragment includes the sequence of SEQ ID NO:47 and the sequence of SEQ ID NO:48. In embodiments, the full length antibody, F(ab)2 fragment, scFv, or Fab′ fragment includes the sequence of SEQ ID NO:49. In embodiments, the full length antibody, F(ab)2 fragment, scFv, or Fab′ fragment includes the sequence of SEQ ID NO:50. In embodiments, the full length antibody, F(ab)2 fragment, scFv, or Fab′ fragment includes the sequence of SEQ ID NO:51. In embodiments, the full length antibody, F(ab)2 fragment, scFv, or Fab′ fragment includes the sequence of SEQ ID NO:52. In embodiments, the full length antibody, F(ab)2 fragment, scFv, or Fab′ fragment includes the sequence of SEQ ID NO:53. In embodiments, the full length antibody, F(ab)2 fragment, scFv, or Fab′ fragment includes the sequence of SEQ ID NO:54. In embodiments, the full length antibody, F(ab)2 fragment, scFv, or Fab′ fragment includes the sequence of SEQ ID NO:55. In embodiments, the full length antibody, F(ab)2 fragment, scFv, or Fab′ fragment includes the sequence of SEQ ID NO:56. In embodiments, the full length antibody, F(ab)2 fragment, scFv, or Fab′ fragment includes the sequence of SEQ ID NO:55 and the sequence of SEQ ID NO:56. In embodiments, the full length antibody, F(ab)2 fragment, scFv, or Fab′ fragment includes the sequence of SEQ ID NO:57. In embodiments, the full length antibody, F(ab)2 fragment, scFv, or Fab′ fragment includes the sequence of SEQ ID NO:58. In embodiments, the full length antibody, F(ab)2 fragment, scFv, or Fab′ fragment includes the sequence of SEQ ID NO:59. In embodiments, the full length antibody, F(ab)2 fragment, scFv, or Fab′ fragment includes the sequence of SEQ ID NO:60. In embodiments, the full length antibody, F(ab)2 fragment, scFv, or Fab′ fragment includes the sequence of SEQ ID NO:61. In embodiments, the full length antibody, F(ab)2 fragment, scFv, or Fab′ fragment includes the sequence of SEQ ID NO:62. In embodiments, the full length antibody, F(ab)2 fragment, scFv, or Fab′ fragment includes the sequence of SEQ ID NO:63. In embodiments, the full length antibody, F(ab)2 fragment, scFv, or Fab′ fragment includes the sequence of SEQ ID NO:64. In embodiments, the full length antibody, F(ab)2 fragment, scFv, or Fab′ fragment includes the sequence of SEQ ID NO:63 and the sequence of SEQ ID NO:64. In embodiments, the full length antibody, F(ab)2 fragment, scFv, or Fab′ fragment includes the sequence of SEQ ID NO:65. In embodiments, the full length antibody, F(ab)2 fragment, scFv, or Fab′ fragment includes the sequence of SEQ ID NO:66. In embodiments, the full length antibody, F(ab)2 fragment, scFv, or Fab′ fragment includes the sequence of SEQ ID NO:67. In embodiments, the full length antibody, F(ab)2 fragment, scFv, or Fab′ fragment includes the sequence of SEQ ID NO:68. In embodiments, the full length antibody, F(ab)2 fragment, scFv, or Fab′ fragment includes the sequence of SEQ ID NO:69. In embodiments, the full length antibody, F(ab)2 fragment, scFv, or Fab′ fragment includes the sequence of SEQ ID NO:70. In embodiments, the full length antibody, F(ab)2 fragment, scFv, or Fab′ fragment includes the sequence of SEQ ID NO:71. In embodiments, the full length antibody, F(ab)2 fragment, scFv, or Fab′ fragment includes the sequence of SEQ ID NO:72. In embodiments, the full length antibody, F(ab)2 fragment, scFv, or Fab′ fragment includes the sequence of SEQ ID NO:71 and the sequence of SEQ ID NO:72. In embodiments, at least a portion of the full length antibody, F(ab)2 fragment, scFv, or Fab′ fragment is encoded by the sequence of SEQ ID NO:73. In embodiments, at least a portion of the full length antibody, F(ab)2 fragment, scFv, or Fab′ fragment is encoded by the sequence of SEQ ID NO:74. In embodiments, at least a portion of the full length antibody, F(ab)2 fragment, scFv, or Fab′ fragment is encoded by the sequence of SEQ ID NO:75. In embodiments, at least a portion of the full length antibody, F(ab)2 fragment, scFv, or Fab′ fragment is encoded by the sequence of SEQ ID NO:76. In embodiments, at least a portion of the full length antibody, F(ab)2 fragment, scFv, or Fab′ fragment is encoded by the sequence of SEQ ID NO:77. In embodiments, at least a portion of the full length antibody, F(ab)2 fragment, scFv, or Fab′ fragment is encoded by the sequence of SEQ ID NO:78. In embodiments, at least a portion of the full length antibody, F(ab)2 fragment, scFv, or Fab′ fragment is encoded by the sequence of SEQ ID NO:79. In embodiments, at least a portion of the full length antibody, F(ab)2 fragment, scFv, or Fab′ fragment is encoded by the sequence of SEQ ID NO:80. In embodiments, a first portion of the full length antibody, F(ab)2 fragment, scFv, or Fab′ fragment is encoded by the sequence of SEQ ID NO:79 and a second portion of the full length antibody, F(ab)2 fragment, scFv, or Fab′ fragment is encoded by the sequence of SEQ ID NO:80. In embodiments, the full length antibody, F(ab)2 fragment, scFv, or Fab′ fragment includes the sequence of SEQ ID NO:81. In embodiments, the full length antibody, F(ab)2 fragment, scFv, or Fab′ fragment includes the sequence of SEQ ID NO:82. In embodiments, the full length antibody, F(ab)2 fragment, scFv, or Fab′ fragment includes the sequence of SEQ ID NO:83. In embodiments, the full length antibody, F(ab)2 fragment, scFv, or Fab′ fragment includes the sequence of SEQ ID NO:84. In embodiments, the full length antibody, F(ab)2 fragment, scFv, or Fab′ fragment includes the sequence of SEQ ID NO:85. In embodiments, the full length antibody, F(ab)2 fragment, scFv, or Fab′ fragment includes the sequence of SEQ ID NO:86. In embodiments, the full length antibody, F(ab)2 fragment, scFv, or Fab′ fragment includes the sequence of SEQ ID NO:87. In embodiments, the full length antibody, F(ab)2 fragment, scFv, or Fab′ fragment includes the sequence of SEQ ID NO:88. In embodiments, the full length antibody, F(ab)2 fragment, scFv, or Fab′ fragment includes the sequence of SEQ ID NO:87 and the sequence of SEQ ID NO:88. In embodiments, the full length antibody, F(ab)2 fragment, scFv, or Fab′ fragment includes the sequence of SEQ ID NO:89. In embodiments, the full length antibody, F(ab)2 fragment, scFv, or Fab′ fragment includes the sequence of SEQ ID NO:90. In embodiments, the full length antibody, F(ab)2 fragment, scFv, or Fab′ fragment includes the sequence of SEQ ID NO:91. In embodiments, the full length antibody, F(ab)2 fragment, scFv, or Fab′ fragment includes the sequence of SEQ ID NO:92. In embodiments, the full length antibody, F(ab)2 fragment, scFv, or Fab′ fragment includes the sequence of SEQ ID NO:93. In embodiments, the full length antibody, F(ab)2 fragment, scFv, or Fab′ fragment includes the sequence of SEQ ID NO:94. In embodiments, the full length antibody, F(ab)2 fragment, scFv, or Fab′ fragment includes the sequence of SEQ ID NO:95. In embodiments, the full length antibody, F(ab)2 fragment, scFv, or Fab′ fragment includes the sequence of SEQ ID NO:96. In embodiments, the full length antibody, F(ab)2 fragment, scFv, or Fab′ fragment includes the sequence of SEQ ID NO:95 and the sequence of SEQ ID NO:96. In embodiments, the full length antibody, F(ab)2 fragment, scFv, or Fab′ fragment includes the sequence of SEQ ID NO:97. In embodiments, the full length antibody, F(ab)2 fragment, scFv, or Fab′ fragment includes the sequence of SEQ ID NO:98. In embodiments, the full length antibody, F(ab)2 fragment, scFv, or Fab′ fragment includes the sequence of SEQ ID NO:99. In embodiments, the full length antibody, F(ab)2 fragment, scFv, or Fab′ fragment includes the sequence of SEQ ID NO:100. In embodiments, the full length antibody, F(ab)2 fragment, scFv, or Fab′ fragment includes the sequence of SEQ ID NO:101. In embodiments, the full length antibody, F(ab)2 fragment, scFv, or Fab′ fragment includes the sequence of SEQ ID NO: 102. In embodiments, the full length antibody, F(ab)2 fragment, scFv, or Fab′ fragment includes the sequence of SEQ ID NO:103. In embodiments, the full length antibody, F(ab)2 fragment, scFv, or Fab′ fragment includes the sequence of SEQ ID NO: 104. In embodiments, the full length antibody, F(ab)2 fragment, scFv, or Fab′ fragment includes the sequence of SEQ ID NO: 103 and the sequence of SEQ ID NO:104. In embodiments, at least a portion of the full length antibody, F(ab)2 fragment, scFv, or Fab′ fragment is encoded by the sequence of SEQ ID NO:105. In embodiments, at least a portion of the full length antibody, F(ab)2 fragment, scFv, or Fab′ fragment is encoded by the sequence of SEQ ID NO: 106. In embodiments, at least a portion of the full length antibody, F(ab)2 fragment, scFv, or Fab′ fragment is encoded by the sequence of SEQ ID NO:107. In embodiments, at least a portion of the full length antibody, F(ab)2 fragment, scFv, or Fab′ fragment is encoded by the sequence of SEQ ID NO: 108. In embodiments, at least a portion of the full length antibody, F(ab)2 fragment, scFv, or Fab′ fragment is encoded by the sequence of SEQ ID NO:109. In embodiments, at least a portion of the full length antibody, F(ab)2 fragment, scFv, or Fab′ fragment is encoded by the sequence of SEQ ID NO:110. In embodiments, at least a portion of the full length antibody, F(ab)2 fragment, scFv, or Fab′ fragment is encoded by the sequence of SEQ ID NO:111. In embodiments, at least a portion of the full length antibody, F(ab)2 fragment, scFv, or Fab′ fragment is encoded by the sequence of SEQ ID NO:112. In embodiments, a first portion of the full length antibody, F(ab)2 fragment, scFv, or Fab′ fragment is encoded by the sequence of SEQ ID NO:111 and a second portion of the full length antibody, F(ab)2 fragment, scFv, or Fab′ fragment is encoded by the sequence of SEQ ID NO:112. In embodiments, the full length antibody, F(ab)2 fragment, scFv, or Fab′ fragment includes the sequence of SEQ ID NO: 113. In embodiments, the full length antibody, F(ab)2 fragment, scFv, or Fab′ fragment includes the sequence of SEQ ID NO:114. In embodiments, the full length antibody, F(ab)2 fragment, scFv, or Fab′ fragment includes the sequence of SEQ ID NO: 115. In embodiments, the full length antibody, F(ab)2 fragment, scFv, or Fab′ fragment includes the sequence of SEQ ID NO: 116. In embodiments, the full length antibody, F(ab)2 fragment, scFv, or Fab′ fragment includes the sequence of SEQ ID NO:117. In embodiments, the full length antibody, F(ab)2 fragment, scFv, or Fab′ fragment includes the sequence of SEQ ID NO: 118. In embodiments, the full length antibody, F(ab)2 fragment, scFv, or Fab′ fragment includes the sequence of SEQ ID NO: 119. In embodiments, the full length antibody, F(ab)2 fragment, scFv, or Fab′ fragment includes the sequence of SEQ ID NO:120. In embodiments, the full length antibody, F(ab)2 fragment, scFv, or Fab′ fragment includes the sequence of SEQ ID NO: 119 and the sequence of SEQ ID NO:120. In embodiments, at least a portion of the full length antibody, F(ab)2 fragment, scFv, or Fab′ fragment is encoded by the sequence of SEQ ID NO:121. In embodiments, at least a portion of the full length antibody, F(ab)2 fragment, scFv, or Fab′ fragment is encoded by the sequence of SEQ ID NO: 122. In embodiments, at least a portion of the full length antibody, F(ab)2 fragment, scFv, or Fab′ fragment is encoded by the sequence of SEQ ID NO:123. In embodiments, at least a portion of the full length antibody, F(ab)2 fragment, scFv, or Fab′ fragment is encoded by the sequence of SEQ ID NO:124. In embodiments, at least a portion of the full length antibody, F(ab)2 fragment, scFv, or Fab′ fragment is encoded by the sequence of SEQ ID NO: 125. In embodiments, at least a portion of the full length antibody, F(ab)2 fragment, scFv, or Fab′ fragment is encoded by the sequence of SEQ ID NO:126. In embodiments, at least a portion of the full length antibody, F(ab)2 fragment, scFv, or Fab′ fragment is encoded by the sequence of SEQ ID NO: 127. In embodiments, at least a portion of the full length antibody, F(ab)2 fragment, scFv, or Fab′ fragment is encoded by the sequence of SEQ ID NO:128. In embodiments, a first portion of the full length antibody, F(ab)2 fragment, scFv, or Fab′ fragment is encoded by the sequence of SEQ ID NO:127 and a second portion of the full length antibody, F(ab)2 fragment, scFv, or Fab′ fragment is encoded by the sequence of SEQ ID NO:128. In embodiments, at least a portion of the full length antibody, F(ab)2 fragment, scFv, or Fab′ fragment is encoded by the sequence of SEQ ID NO:193. In embodiments, at least a portion of the full length antibody, F(ab)2 fragment, scFv, or Fab′ fragment is encoded by the sequence of SEQ ID NO: 194. In embodiments, a first portion of the full length antibody, F(ab)2 fragment, scFv, or Fab′ fragment is encoded by the sequence of SEQ ID NO:193 and a second portion of the full length antibody, F(ab)2 fragment, scFv, or Fab′ fragment is encoded by the sequence of SEQ ID NO: 194. Chimeric AT19 family antibodies described herein (e.g., “M20c”, which is also referenced herein as “AT19.20c”) and mouse AT19 family antibodies described herein (e.g., “M20” referenced in FIG. 1) can comprise the same mouse CDR amino acid sequences and/or the same mouse variable region amino acid sequences (e.g., AT-19.20 sequences described herein, which can include SEQ ID NOs: 9-16). Humanized AT19 family antibodies described herein (e.g., “M20hv1”, “M20hv2”, “M20hv3”, “M20hv4”) can comprise CDR amino acid sequences and/or variable region sequences distinct from the mouse and chimeric versions of the same antibody clone. For instance, AT-19.20 h.v1 (also referred herein to as M20hv1) can comprise one or more of SEQ ID NOs: 17-24, AT-19.20 h.v2 (also referred herein as M20hv2 or “M20”) can comprise one or more of SEQ ID NOs: 25-32, AT-19.20 h.v3 (also referred herein to as M20hv3) can comprise one or more of SEQ ID NOs: 33-40, and AT-19.20 h.v4 (also referred herein as M20hv4) can comprise one or more of SEQ ID NOs: 41-48.

In embodiments, the antibody is an IgG. In embodiments, the antibody is an IgG1. In embodiments, the antibody is a human IgG.

In embodiments, an antibody is capable of binding FLT3 or a portion thereof. In embodiments, the antibody is bound to FLT3 or a portion thereof. In embodiments, the FLT3 forms part of a cell. In embodiments, the antibody is attached to a detectable label.

In one embodiment of a first antibody, the light chain variable domain includes: a CDR L1 as set forth in SEQ ID NO:1, a CDR L2 as set forth in SEQ ID NO:2, a CDR L3 as set forth in SEQ ID NO:3, or a light chain variable domain sequence as set forth in SEQ ID NO:7, and the heavy chain variable domain includes: a CDR H1 as set forth in SEQ ID NO:4, a CDR H2 as set forth in SEQ ID NO:5, a CDR H3 as set forth in SEQ ID NO:6, or a heavy chain variable domain sequence as set forth in SEQ ID NO:8. In one further embodiment, the first antibody is antibody AT-19.10.

In one embodiment of a first antibody, the light chain variable domain includes: a CDR L1 as set forth in SEQ ID NO:9, a CDR L2 as set forth in SEQ ID NO:10, a CDR L3 as set forth in SEQ ID NO:11, or a light chain variable domain sequence as set forth in SEQ ID NO:15, and the heavy chain variable domain includes: a CDR H1 as set forth in SEQ ID NO:12, a CDR H2 as set forth in SEQ ID NO:13, a CDR H3 as set forth in SEQ ID NO:14, or a heavy chain variable domain sequence as set forth in SEQ ID NO: 16. In one further embodiment, the first antibody is antibody AT-19.20.

In one embodiment of a first antibody, the light chain variable domain includes: a CDR L1 as set forth in SEQ ID NO:17, a CDR L2 as set forth in SEQ ID NO:18, a CDR L3 as set forth in SEQ ID NO:19, or a light chain variable domain sequence as set forth in SEQ ID NO:23, and the heavy chain variable domain includes: a CDR H1 as set forth in SEQ ID NO:20, a CDR H2 as set forth in SEQ ID NO:21, a CDR H3 as set forth in SEQ ID NO:22, or a heavy chain variable domain sequence as set forth in SEQ ID NO:24. In one further embodiment, the first antibody is antibody AT-19.20 h.v1.

In one embodiment of a first antibody, the light chain variable domain includes: a CDR L1 as set forth in SEQ ID NO:25, a CDR L2 as set forth in SEQ ID NO:26, a CDR L3 as set forth in SEQ ID NO:27, or a light chain variable domain sequence as set forth in SEQ ID NO:31, and the heavy chain variable domain includes: a CDR H1 as set forth in SEQ ID NO:28, a CDR H2 as set forth in SEQ ID NO:29, a CDR H3 as set forth in SEQ ID NO:30, or a heavy chain variable domain sequence as set forth in SEQ ID NO:32. In one further embodiment, the first antibody is antibody AT-19.20 h.v2.

In one embodiment of a first antibody, the light chain variable domain includes: a CDR L1 as set forth in SEQ ID NO:33, a CDR L2 as set forth in SEQ ID NO:34, a CDR L3 as set forth in SEQ ID NO:35, or a light chain variable domain sequence as set forth in SEQ ID NO:39, and the heavy chain variable domain includes: a CDR H1 as set forth in SEQ ID NO:36, a CDR H2 as set forth in SEQ ID NO:37, a CDR H3 as set forth in SEQ ID NO:38, or a heavy chain variable domain sequence as set forth in SEQ ID NO:40. In one further embodiment, the first antibody is antibody AT-19.20 h.v3.

In one embodiment of a first antibody, the light chain variable domain includes: a CDR L1 as set forth in SEQ ID NO:41, a CDR L2 as set forth in SEQ ID NO:42, a CDR L3 as set forth in SEQ ID NO:43, or a light chain variable domain sequence as set forth in SEQ ID NO:47, and the heavy chain variable domain includes: a CDR H1 as set forth in SEQ ID NO:44, a CDR H2 as set forth in SEQ ID NO:45, a CDR H3 as set forth in SEQ ID NO:46, or a heavy chain variable domain sequence as set forth in SEQ ID NO:48. In one further embodiment, the first antibody is antibody AT-19.20 h.v4.

In one embodiment of a first antibody, the light chain variable domain includes: a CDR L1 as set forth in SEQ ID NO:49, a CDR L2 as set forth in SEQ ID NO:50, a CDR L3 as set forth in SEQ ID NO:51, or a light chain variable domain sequence as set forth in SEQ ID NO:55, and the heavy chain variable domain includes: a CDR H1 as set forth in SEQ ID NO:52, a CDR H2 as set forth in SEQ ID NO:53, a CDR H3 as set forth in SEQ ID NO:54, or a heavy chain variable domain sequence as set forth in SEQ ID NO:55. In one further embodiment, the first antibody is antibody AT-19.21.

In one embodiment of a first antibody, the light chain variable domain includes: a CDR L1 as set forth in SEQ ID NO:57, a CDR L2 as set forth in SEQ ID NO:58, a CDR L3 as set forth in SEQ ID NO:59, or a light chain variable domain sequence as set forth in SEQ ID NO:63, and the heavy chain variable domain includes: a CDR H1 as set forth in SEQ ID NO:60, a CDR H2 as set forth in SEQ ID NO:61, a CDR H3 as set forth in SEQ ID NO:62, or a heavy chain variable domain sequence as set forth in SEQ ID NO:64. In one further embodiment, the first antibody is antibody AT-19.38.

In one embodiment of a first antibody, the light chain variable domain includes: a CDR L1 as set forth in SEQ ID NO:65, a CDR L2 as set forth in SEQ ID NO:66, a CDR L3 as set forth in SEQ ID NO:67, or a light chain variable domain sequence as set forth in SEQ ID NO:71, and the heavy chain variable domain includes: a CDR H1 as set forth in SEQ ID NO:68, a CDR H2 as set forth in SEQ ID NO:69, a CDR H3 as set forth in SEQ ID NO:70, or a heavy chain variable domain sequence as set forth in SEQ ID NO:72. In one further embodiment, the first antibody is antibody AT-19.42.

In one embodiment of a first antibody, the light chain variable domain includes: a CDR L1 as set forth in SEQ ID NO:73, a CDR L2 as set forth in SEQ ID NO:74, a CDR L3 as set forth in SEQ ID NO:75, or a light chain variable domain sequence as set forth in SEQ ID NO:79, and the heavy chain variable domain includes: a CDR H1 as set forth in SEQ ID NO:76, a CDR H2 as set forth in SEQ ID NO:77, a CDR H3 as set forth in SEQ ID NO:78, or a heavy chain variable domain sequence as set forth in SEQ ID NO:80. In one further embodiment, the first antibody is antibody AT-19.80.

In one embodiment of a first antibody, the light chain variable domain includes: a CDR L1 as set forth in SEQ ID NO:81, a CDR L2 as set forth in SEQ ID NO:82, a CDR L3 as set forth in SEQ ID NO:83, or a light chain variable domain sequence as set forth in SEQ ID NO:87, and the heavy chain variable domain includes: a CDR H1 as set forth in SEQ ID NO:84, a CDR H2 as set forth in SEQ ID NO:85, a CDR H3 as set forth in SEQ ID NO:86, or a heavy chain variable domain sequence as set forth in SEQ ID NO:88. In one further embodiment, the first antibody is antibody AT-19.84.

In one embodiment of a first antibody, the light chain variable domain includes: a CDR L1 as set forth in SEQ ID NO:89, a CDR L2 as set forth in SEQ ID NO:90, a CDR L3 as set forth in SEQ ID NO:91, or a light chain variable domain sequence as set forth in SEQ ID NO:95, and the heavy chain variable domain includes: a CDR H1 as set forth in SEQ ID NO:92, a CDR H2 as set forth in SEQ ID NO:93, a CDR H3 as set forth in SEQ ID NO:94, or a heavy chain variable domain sequence as set forth in SEQ ID NO:96. In one further embodiment, the first antibody is antibody AT-19.88.

In one embodiment of a first antibody, the light chain variable domain includes: a CDR L1 as set forth in SEQ ID NO:97, a CDR L2 as set forth in SEQ ID NO:98, a CDR L3 as set forth in SEQ ID NO:99, or a light chain variable domain sequence as set forth in SEQ ID NO:103, and the heavy chain variable domain includes: a CDR H1 as set forth in SEQ ID NO:100, a CDR H2 as set forth in SEQ ID NO:101, a CDR H3 as set forth in SEQ ID NO:102, or a heavy chain variable domain sequence as set forth in SEQ ID NO:104. In one further embodiment, the first antibody is antibody B1.

In one embodiment of a first antibody, the light chain variable domain includes: a CDR L1 as set forth in SEQ ID NO: 105, a CDR L2 as set forth in SEQ ID NO: 106, a CDR L3 as set forth in SEQ ID NO: 107, or a light chain variable domain sequence as set forth in SEQ ID NO:111, and the heavy chain variable domain includes: a CDR H1 as set forth in SEQ ID NO:108, a CDR H2 as set forth in SEQ ID NO: 109, a CDR H3 as set forth in SEQ ID NO:110, or a heavy chain variable domain sequence as set forth in SEQ ID NO:112. In one further embodiment, the first antibody is antibody B32.

In one embodiment of a first antibody, the light chain variable domain includes: a CDR L1 as set forth in SEQ ID NO: 113, a CDR L2 as set forth in SEQ ID NO: 114, a CDR L3 as set forth in SEQ ID NO: 115, or a light chain variable domain sequence as set forth in SEQ ID NO:119, and the heavy chain variable domain includes: a CDR H1 as set forth in SEQ ID NO:116, a CDR H2 as set forth in SEQ ID NO:117, a CDR H3 as set forth in SEQ ID NO:118, or a heavy chain variable domain sequence as set forth in SEQ ID NO:120. In one further embodiment, the first antibody is antibody 16F7.

In one embodiment of a first antibody, the light chain variable domain includes: a CDR L1 as set forth in SEQ ID NO:121, a CDR L2 as set forth in SEQ ID NO: 122, a CDR L3 as set forth in SEQ ID NO: 123, or a light chain variable domain sequence as set forth in SEQ ID NO:127, and the heavy chain variable domain includes: a CDR H1 as set forth in SEQ ID NO:124, a CDR H2 as set forth in SEQ ID NO: 125, a CDR H3 as set forth in SEQ ID NO:126, or a heavy chain variable domain sequence as set forth in SEQ ID NO:128. In one further embodiment, the first antibody is antibody 7F10.

In one embodiment of a first antibody, the light chain variable domain includes: a light chain variable domain sequence as set forth in SEQ ID NO: 193, and a heavy chain variable domain sequence as set forth in SEQ ID NO:194. In one further embodiment, the first antibody is antibody ALT-Anti-Flt3 (also referred to herein as AT-19 E10 or E10).

### Recombinant Protein Compositions

As described above, the light chain variable (VL) domain and the heavy chain variable (VH) domain provided herein including embodiments thereof, may each independently form part of an antibody, an antibody variant, a fragment of an antibody, a fragment of an antibody variant, or a recombinant protein (e.g., an scFv antibody, a bispecific antibody). Provided herein are, inter alia, recombinant proteins (e.g., scFv antibodies), which include the light chain variable (VL) domain and/or the heavy chain variable (VH) domain as provided herein and are therefore capable of binding FLT3, thereby effectively inhabiting FLT3 activity. In embodiments, the recombinant protein is a scFv. In embodiments, the recombinant protein is a bispecific antibody.

### Nucleic Acid Compositions

The compositions provided herein include nucleic acid molecules encoding anti-FLT3 antibodies and recombinant proteins provided herein including embodiments thereof. Thus, in an aspect, an isolated nucleic acid encoding an antibody as provided herein including embodiments thereof is provided. Examples of such nucleic acids are provided in Table 2.

In another aspect, an isolated nucleic acid encoding a recombinant protein as provided herein, including embodiments thereof, is provided.

In some aspects, a cell can comprise an isolated nucleic acid encoding a (e.g., recombinant) protein as provided herein, for example, wherein the protein is an antibody or fragment thereof described herein, such as an anti-FLT3 antibody.

### Cell Compositions

The antibodies provided herein may be used as immunotherapeutic agents. In an aspect a cell including an antibody provided herein including embodiments thereof is provided. In another aspect, a cell including a nucleic acid encoding an antibody provided herein including embodiments thereof is provided. In embodiments, the cell can be a lymphoid cell, a myeloid cell, or a stem cell. In embodiments, the (e.g., lymphoid) cell is a T cell or a B cell. In embodiments, the cell is a T cell. In embodiments, the cell is a B cell. In embodiments, the cell is a FLT3 (e.g., high) expressing cell.

### Pharmaceutical Compositions

The compositions provided herein include pharmaceutical compositions including an anti-FLT3 antibody provided herein including embodiments thereof. Thus, in an aspect is provided a pharmaceutical composition including a therapeutically effective amount of an antibody provided herein including embodiments thereof or a therapeutically effective amount of the isolated nucleic acid provided herein including embodiments thereof; and a pharmaceutically acceptable excipient.

### Methods

The compositions (e.g., anti-FLT3 antibodies and recombinant proteins) provided herein, including embodiments thereof, are contemplated as providing effective treatments for diseases such as cancer. Thus, in an aspect is provided a method of treating cancer in a subject in need thereof, the method including administering to a subject a therapeutically effective amount of an antibody as provided herein including embodiments thereof, thereby treating cancer in the subject.

In another aspect is provided a method of treating cancer in a subject in need thereof, the method including administering to a subject a therapeutically effective amount of a recombinant protein as described herein, including embodiments thereof, thereby treating cancer in the subject. In some aspects, a composition described herein (e.g., an anti-FLT3 antibody and recombinant proteins described herein) can be used to treat a plurality of cancer types.

In embodiments, the cancer is acute myeloid leukemia (AML), acute lymphoblastic leukemia (ALL), lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, renal cancer, melanoma, breast cancer, neuroblastoma, or glioblastoma. In embodiments, the cancer is acute myeloid leukemia. In embodiments, the cancer is acute lymphoblastic leukemia (ALL). In embodiments, the cancer is breast cancer. In embodiments, the cancer is pancreatic cancer. In embodiments, the cancer is lung cancer. In embodiments, the lung cancer is non-small cell lung cancer. In embodiments, the cancer is ovarian cancer. In embodiments, the cancer is a glioblastoma. In embodiments, the cancer is colorectal cancer. In embodiments, the cancer is renal cancer.

In some cases, an anti-FLT3 antibody can be useful in the treatment of one or more cancers or tumors selected from one or more of: acute myeloid leukemia (AML), acute lymphoblastic leukemia (ALL), lung cancer (e.g., non-small cell lung cancer), breast cancer, ovarian cancer, renal cancer, glioblastoma, pancreatic cancer, and/or colorectal cancer. In some cases, an anti-FLT3 antibody can be useful in reducing adhesion of cells from one or more of: acute myeloid leukemia (AML), acute lymphoblastic leukemia (ALL), a lung cancer (e.g., non-small cell lung cancer), a breast cancer, an ovarian cancer, a renal cancer, glioblastoma, pancreatic cancer, and/or a colorectal cancer (e.g., compared to a control treatment, which may include a vehicle control, a placebo control, or no treatment). In some cases, an anti-FLT3 antibody can be useful in reducing proliferation of cells from one or more of: acute myeloid leukemia (AML), acute lymphoblastic leukemia (ALL), a lung cancer (e.g., non-small cell lung cancer), a breast cancer, an ovarian cancer, a renal cancer, glioblastoma, pancreatic cancer, and/or a colorectal cancer (e.g., compared to a control treatment, which may include a vehicle control, a placebo control, or no treatment). In some cases, an anti-FLT3 antibody can be useful in inhibiting migration (or metastasis) of cells from one or more of: acute myeloid leukemia (AML), acute lymphoblastic leukemia (ALL), a lung cancer (e.g., non-small cell lung cancer), a breast cancer, an ovarian cancer, a renal cancer, glioblastoma, pancreatic cancer, and/or a colorectal cancer (e.g., compared to a control treatment, which may include a vehicle control, a placebo control, or no treatment). In some cases, an anti-FLT3 antibody can be useful in decreasing secretion of a chemokine (e.g., interleukin-6) in a cell from one or more of: acute myeloid leukemia (AML), acute lymphoblastic leukemia (ALL), a lung cancer (e.g., non-small cell lung cancer), a breast cancer, an ovarian cancer, a renal cancer, glioblastoma, pancreatic cancer, and/or a colorectal cancer (e.g., compared to a control treatment, which may include a vehicle control, a placebo control, or no treatment). In some cases, an anti-FLT3 antibody can be useful in increasing peripheral mononuclear blood cell viability in a subject having one or more of: acute myeloid leukemia (AML), acute lymphoblastic leukemia (ALL), a lung cancer (e.g., non-small cell lung cancer), a breast cancer, an ovarian cancer, a renal cancer, glioblastoma, pancreatic cancer, and/or a colorectal cancer (e.g., compared to a control treatment, which may include a vehicle control, a placebo control, or no treatment). In some cases, an anti-FLT3 antibody can modulate (e.g., endogenous) FLT3 signaling, which may have the effect of abrogating or eliminating endogenous immune cell-mediated reductions in tumor or cancer cell proliferation, migration, or pro-inflammatory secretion (e.g., as compared to control treatment, which may include a vehicle control, a placebo control, or no treatment).

In embodiments, the method further includes administering to the subject a second therapeutic agent. In embodiments, the method further includes administering a therapeutically effective amount of an antineoplastic agent. In embodiments, the effective amount of an antibody and the effective amount of an antineoplastic agent are a combined synergistic amount. In embodiments, an antineoplastic agent can comprise a chemotherapeutic drug, such as N-acetyl-gamma-calicheamicin, pyrrolobenzodiazepine dimer, monomethyl auristatin E (MMAE), DXd, or DX8951 (e.g., DX-8951).

In embodiments, the antibody and the antineoplastic agent are administered sequentially or concurrently. In embodiments, the antibody and the antineoplastic agent are administered sequentially. In embodiments, the antibody and the antineoplastic agent are administered concurrently. In embodiments, the antibody and the antineoplastic agent are admixed together prior to administration. In embodiments, the antibody and the antineoplastic agent are administered in a single dosage form. In embodiments, the antibody and the antineoplastic agent are administered in two separate dosage forms.

In an aspect is provided a method of treating cancer in a subject in need thereof, the method including administering to a subject a combined effective amount of an antibody as provided herein including embodiments thereof and a antineoplastic agent, thereby treating cancer in the subject. In embodiments, the combined effective amount is a combined synergistic amount.

In embodiments, the effective amount of an antibody provided herein including embodiments thereof, and the effective amount of an antineoplastic agent are a combined synergistic amount. A “combined synergistic amount” as used herein refers to the sum of a first amount (e.g., an amount of an antibody provided herein including embodiments thereof) and a second amount (e.g., an amount of an antineoplastic agent), that results in a synergistic effect (i.e. an effect greater than an additive effect). Therefore, the terms “synergy”, “synergism”, “synergistic”, “combined synergistic amount”, and “synergistic therapeutic effect” which are used herein interchangeably, refer to a measured effect of compounds administered in combination where the measured effect is greater than the sum of the individual effects of each of the compounds administered alone as a single agent. In embodiments, the measured effect of the compounds (e.g., the antibody provided herein and the antineoplastic agent) administered is 1-fold, 1.5-fold, 2-fold, 3-fold, 4-fold, 5-fold, 10-fold or greater than the sum of the individual effects of each of the compounds administered alone as a single agent.

In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the antibody provided herein when used separately from the antineoplastic agent. In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the antineoplastic agent when used separately from the antibody provided herein.

In embodiments, the antibody is administered intratumorally, intravenously, subcutaneously, intraperitoneally, intradermally, or intramuscularly (e.g., in pharmaceutically acceptable formulation).

In embodiments, the antibody is administered at an amount from about 10 micrograms per deciliter (ug/dL) to about 2000 ug/dL. In embodiments, the antibody is administered at an amount from about 10 ug/dL to about 1500 ug/dL, from about 10 ug/dL to about 1000 ug/dL, from about 10 ug/dL to about 900 ug/dL, from about 10 ug/dL to about 800 ug/dL, from about 10 ug/dL to about 700 ug/dL, from about 10 ug/dL to about 600 ug/dL, from about 10 ug/dL to about 500 ug/dL, from about 10 ug/dL to about 400 ug/dL, from about 10 ug/dL to about 300 ug/dL, from about 10 ug/dL to about 200 ug/dL, from about 10 ug/dL to about 100 ug/dL, from about 10 ug/dL to about 50 ug/dL, from about 50 ug/dL to about 2000 ug/dL, from about 50 ug/dL to about 1500 ug/dL, from about 50 ug/dL to about 1000 ug/dL, from about 50 ug/dL to about 900 ug/dL, from about 50 ug/dL to about 800 ug/dL, from about 50 ug/dL to about 700 ug/dL, from about 50 ug/dL to about 600 ug/dL, from about 50 ug/dL to about 500 ug/dL, from about 50 ug/dL to about 400 ug/dL, from about 50 ug/dL to about 300 ug/dL, from about 50 ug/dL to about 200 ug/dL, from about 50 ug/dL to about 100 ug/dL, from about 100 ug/dL to about 2000 ug/dL, from about 100 ug/dL to about 1500 ug/dL, from about 100 ug/dL to about 1000 ug/dL, from about 100 ug/dL to about 900 ug/dL, from about 100 ug/dL to about 800 ug/dL, from about 100 ug/dL to about 700 ug/dL, from about 100 ug/dL to about 600 ug/dL, from about 100 ug/dL to about 500 ug/dL, from about 100 ug/dL to about 400 ug/dL, from about 100 ug/dL to about 300 ug/dL, from about 100 ug/dL to about 200 ug/dL, from about 200 ug/dL to about 2000 ug/dL, from about 200 ug/dL to about 1500 ug/dL, from about 200 ug/dL to about 1000 ug/dL, from about 200 ug/dL to about 900 ug/dL, from about 200 ug/dL to about 800 ug/dL, from about 200 ug/dL to about 700 ug/dL, from about 200 ug/dL to about 600 ug/dL, from about 200 ug/dL to about 500 ug/dL, from about 200 ug/dL to about 400 ug/dL, from about 200 ug/dL to about 300 ug/dL, from about 300 ug/dL to about 2000 ug/dL, from about 300 ug/dL to about 1500 ug/dL, from about 300 ug/dL to about 1000 ug/dL, from about 300 ug/dL to about 900 ug/dL, from about 300 ug/dL to about 800 ug/dL, from about 300 ug/dL to about 700 ug/dL, from about 300 ug/dL to about 600 ug/dL, from about 300 ug/dL to about 500 ug/dL, from about 300 ug/dL to about 400 ug/dL, from about 400 ug/dL to about 2000 ug/dL, from about 400 ug/dL to about 1500 ug/dL, from about 400 ug/dL to about 1000 ug/dL, from about 400 ug/dL to about 900 ug/dL, from about 400 ug/dL to about 800 ug/dL, from about 400 ug/dL to about 700 ug/dL, from about 400 ug/dL to about 600 ug/dL, from about 400 ug/dL to about 500 ug/dL, from about 500 ug/dL to about 2000 ug/dL, from about 500 ug/dL to about 1500 ug/dL, from about 500 ug/dL to about 1000 ug/dL, from about 500 ug/dL to about 900 ug/dL, from about 500 ug/dL to about 800 ug/dL, from about 500 ug/dL to about 700 ug/dL, from about 500 ug/dL to about 600 ug/dL, from about 600 ug/dL to about 2000 ug/dL, from about 600 ug/dL to about 1500 ug/dL, from about 600 ug/dL to about 1000 ug/dL, from about 600 ug/dL to about 900 ug/dL, from about 600 ug/dL to about 800 ug/dL, from about 600 ug/dL to about 700 ug/dL, from about 700 ug/dL to about 2000 ug/dL, from about 700 ug/dL to about 1500 ug/dL, from about 700 ug/dL to about 1000 ug/dL, from about 700 ug/dL to about 900 ug/dL, from about 700 ug/dL to about 800 ug/dL, from about 800 ug/dL to about 2000 ug/dL, from about 800 ug/dL to about 1500 ug/dL, from about 800 ug/dL to about 1000 ug/dL, from about 800 ug/dL to about 900 ug/dL, from about 900 ug/dL to about 2000 ug/dL, from about 900 ug/dL to about 1500 ug/dL, from about 900 ug/dL to about 1000 ug/dL, from about 1000 ug/dL to about 2000 ug/dL, from about 1000 ug/dL to about 1500 ug/dL, or from about 1500 ug/dL to about 2000 ug/dL. In embodiments, the antibody is administered at an amount of at least about 10 ug/dL, at least about 50 ug/dL, at least about 100 ug/dL, at least about 200 ug/dL, at least about 300 ug/dL, at least about 400 ug/dL, at least about 500 ug/dL, at least about 600 ug/dL, at least about 700 ug/dL, at least about 800 ug/dL, at least about 900 ug/dL, at least about 1000 ug/dL, at least about 1500 ug/dL, or at least about 2000 ug/dL. In embodiments, the antibody is administered at an amount of at most about 10 ug/dL, at most about 50 ug/dL, at most about 100 ug/dL, at most about 200 ug/dL, at most about 300 ug/dL, at most about 400 ug/dL, at most about 500 ug/dL, at most about 600 ug/dL, at most about 700 ug/dL, at most about 800 ug/dL, at most about 900 ug/dL, at most about 1000 ug/dL, at most about 1500 ug/dL, or at most about 2000 ug/dL. In embodiments, the antibody is administered at an amount of about 10 ug/dL, about 50 ug/dL, about 100 ug/dL, about 200 ug/dL, about 300 ug/dL, about 400 ug/dL, about 500 ug/dL, about 600 ug/dL, about 700 ug/dL, about 800 ug/dL, about 900 ug/dL, about 1000 ug/dL, about 1500 ug/dL, or about 2000 ug/dL.

In embodiments, the antibody is administered at an amount from about 0.01 nM to about 10 nM. In embodiments, the antibody is administered at an amount from about 0.05 nM to about 10 nM. In embodiments, the antibody is administered at an amount from about 0.1 nM to about 10 nM. In embodiments, the antibody is administered at an amount from about 0.5 nM to about 10 nM. In embodiments, the antibody is administered at an amount from about 1 nM to about 10 nM. In embodiments, the antibody is administered at an amount from about 2 nM to about 10 nM. In embodiments, the antibody is administered at an amount from about 4 nM to about 10 nM. In embodiments, the antibody is administered at an amount from about 6 nM to about 10 nM. In embodiments, the antibody is administered at an amount from about 4 nM to about 10 nM. In embodiments, the antibody is administered at an amount from about 8 nM to about 10 nM. In embodiments, the antibody is administered at an amount of about 0.01 nM, 0.05 nM, 0.1 nM, 0.5 nM, 1 nM, 2 nM, 2 nM, 4 nM, 5 nM, 6 nM, 7 nM, 8 nM, 9 nM or 10 nM.

In embodiments, the antibody is administered at an amount from 0.01 nM to 10 nM. In embodiments, the antibody is administered at an amount from 0.05 nM to 10 nM. In embodiments, the antibody is administered at an amount from 0.1 nM to 10 nM. In embodiments, the antibody is administered at an amount from 0.5 nM to 10 nM. In embodiments, the antibody is administered at an amount from 1 nM to 10 nM. In embodiments, the antibody is administered at an amount from 2 nM to 10 nM. In embodiments, the antibody is administered at an amount from 4 nM to 10 nM. In embodiments, the antibody is administered at an amount from 6 nM to 10 nM. In embodiments, the antibody is administered at an amount from 4 nM to 10 nM. In embodiments, the antibody is administered at an amount from 8 nM to 10 nM. In embodiments, the antibody is administered at an amount of 0.01 nM, 0.05 nM, 0.1 nM, 0.5 nM, 1 nM, 2 nM, 2 nM, 4 nM, 5 nM, 6 nM, 7 nM, 8 nM, 9 nM or 10 nM.

In embodiments, the antibody is administered at an amount from about 0.01 nM to about 8 nM. In embodiments, the antibody is administered at an amount from about 0.05 nM to about 8 nM. In embodiments, the antibody is administered at an amount from about 0.1 nM to about 8 nM. In embodiments, the antibody is administered at an amount from about 0.5 nM to about 8 nM. In embodiments, the antibody is administered at an amount from about 1 nM to about 8 nM. In embodiments, the antibody is administered at an amount from about 2 nM to about 8 nM. In embodiments, the antibody is administered at an amount from about 4 nM to about 8 nM. In embodiments, the antibody is administered at an amount from about 6 nM to about 8 nM. In embodiments, the antibody is administered at an amount from about 4 nM to about 8 nM.

In embodiments, the antibody is administered at an amount from 0.01 nM to 8 nM. In embodiments, the antibody is administered at an amount from 0.05 nM to 8 nM. In embodiments, the antibody is administered at an amount from 0.1 nM to 8 nM. In embodiments, the antibody is administered at an amount from 0.5 nM to 8 nM. In embodiments, the antibody is administered at an amount from 1 nM to 8 nM. In embodiments, the antibody is administered at an amount from 2 nM to 8 nM. In embodiments, the antibody is administered at an amount from 4 nM to 8 nM. In embodiments, the antibody is administered at an amount from 6 nM to 8 nM. In embodiments, the antibody is administered at an amount from 4 nM to 8 nM.

In embodiments, the antibody is administered at an amount from about 0.01 nM to about 6 nM. In embodiments, the antibody is administered at an amount from about 0.05 nM to about 6 nM. In embodiments, the antibody is administered at an amount from about 0.1 nM to about 6 nM. In embodiments, the antibody is administered at an amount from about 0.5 nM to about 8 nM. In embodiments, the antibody is administered at an amount from about 1 nM to about 6 nM. In embodiments, the antibody is administered at an amount from about 2 nM to about 6 nM. In embodiments, the antibody is administered at an amount from about 4 nM to about 6 nM.

In embodiments, the antibody is administered at an amount from 0.01 nM to 6 nM. In embodiments, the antibody is administered at an amount from 0.05 nM to 6 nM. In embodiments, the antibody is administered at an amount from 0.1 nM to 6 nM. In embodiments, the antibody is administered at an amount from 0.5 nM to 6 nM. In embodiments, the antibody is administered at an amount from 1 nM to 6 nM. In embodiments, the antibody is administered at an amount from 2 nM to 6 nM. In embodiments, the antibody is administered at an amount from 4 nM to 6 nM.

In embodiments, the antibody is administered at an amount from about 0.01 nM to about 4 nM. In embodiments, the antibody is administered at an amount from about 0.05 nM to about 4 nM. In embodiments, the antibody is administered at an amount from about 0.1 nM to about 4 nM. In embodiments, the antibody is administered at an amount from about 0.5 nM to about 4 nM. In embodiments, the antibody is administered at an amount from about 1 nM to about 4 nM. In embodiments, the antibody is administered at an amount from about 2 nM to about 4 nM.

In embodiments, the antibody is administered at an amount from 0.01 nM to 4 nM. In embodiments, the antibody is administered at an amount from 0.05 nM to 4 nM. In embodiments, the antibody is administered at an amount from 0.1 nM to 4 nM. In embodiments, the antibody is administered at an amount from 0.5 nM to 4 nM. In embodiments, the antibody is administered at an amount from 1 nM to 4 nM. In embodiments, the antibody is administered at an amount from 2 nM to 4 nM.

In embodiments, the antibody is administered at an amount from about 0.01 nM to about 2 nM. In embodiments, the antibody is administered at an amount from about 0.05 nM to about 2 nM. In embodiments, the antibody is administered at an amount from about 0.1 nM to about 2 nM. In embodiments, the antibody is administered at an amount from about 0.5 nM to about 2 nM. In embodiments, the antibody is administered at an amount from about 1 nM to about 2 nM.

In embodiments, the antibody is administered at an amount from 0.01 nM to 2 nM. In embodiments, the antibody is administered at an amount from 0.05 nM to 2 nM. In embodiments, the antibody is administered at an amount from 0.1 nM to 2 nM. In embodiments, the antibody is administered at an amount from 0.5 nM to 2 nM. In embodiments, the antibody is administered at an amount from 1 nM to 2 nM.

In embodiments, the antibody is administered at an amount from about 0.01 nM to about 1 nM. In embodiments, the antibody is administered at an amount from about 0.05 nM to about 1 nM. In embodiments, the antibody is administered at an amount from about 0.1 nM to about 1 nM. In embodiments, the antibody is administered at an amount from about 0.5 nM to about 1 nM.

In embodiments, the antibody is administered at an amount from 0.01 nM to 1 nM. In embodiments, the antibody is administered at an amount from 0.05 nM to 1 nM. In embodiments, the antibody is administered at an amount from 0.1 nM to 1 nM. In embodiments, the antibody is administered at an amount from 0.5 nM to 1 nM.

It is understood that the recombinant protein (i.e., the bispecific antibody or scFv antibody) provided herein including embodiments thereof may be administered at any of the concentrations described herein for the administration of the antibody (e.g., 0.01 nM-10 nM).

In embodiments, the antibody is administered at an amount from about 10 μg to about 500 μg. In embodiments, the antibody is administered at an amount from about 20 μg to about 500 μg. In embodiments, the antibody is administered at an amount from about 30 μg to about 500 μg. In embodiments, the antibody is administered at an amount from about 40 μg to about 500 μg. In embodiments, the antibody is administered at an amount from about 50 μg to about 500 μg. In embodiments, the antibody is administered at an amount from about 60 μg to about 500 μg. In embodiments, the antibody is administered at an amount from about 70 μg to about 500 μg. In embodiments, the antibody is administered at an amount from about 80 μg to about 500 μg. In embodiments, the antibody is administered at an amount from about 90 μg to about 500 μg. In embodiments, the antibody is administered at an amount from about 100 μg to about 500 μg.

In embodiments, the antibody is administered at an amount from about 110 μg to about 500 μg. In embodiments, the antibody is administered at an amount from about 120 μg to about 500 μg. In embodiments, the antibody is administered at an amount from about 130 μg to about 500 μg. In embodiments, the antibody is administered at an amount from about 140 μg to about 500 μg. In embodiments, the antibody is administered at an amount from about 150 μg to about 500 μg. In embodiments, the antibody is administered at an amount from about 160 μg to about 500 μg. In embodiments, the antibody is administered at an amount from about 170 μg to about 500 μg. In embodiments, the antibody is administered at an amount from about 180 μg to about 500 μg. In embodiments, the antibody is administered at an amount from about 190 μg to about 500 μg. In embodiments, the antibody is administered at an amount from about 200 μg to about 500 μg.

In embodiments, the antibody is administered at an amount from about 210 μg to about 500 μg. In embodiments, the antibody is administered at an amount from about 220 μg to about 500 μg. In embodiments, the antibody is administered at an amount from about 230 μg to about 500 μg. In embodiments, the antibody is administered at an amount from about 240 μg to about 500 μg. In embodiments, the antibody is administered at an amount from about 250 μg to about 500 μg. In embodiments, the antibody is administered at an amount from about 260 μg to about 500 μg. In embodiments, the antibody is administered at an amount from about 270 μg to about 500 μg. In embodiments, the antibody is administered at an amount from about 280 μg to about 500 μg. In embodiments, the antibody is administered at an amount from about 290 μg to about 500 μg. In embodiments, the antibody is administered at an amount from about 300 μg to about 500 μg.

In embodiments, the antibody is administered at an amount from about 310 μg to about 500 μg. In embodiments, the antibody is administered at an amount from about 320 μg to about 500 μg. In embodiments, the antibody is administered at an amount from about 330 μg to about 500 μg. In embodiments, the antibody is administered at an amount from about 340 μg to about 500 μg. In embodiments, the antibody is administered at an amount from about 350 μg to about 500 μg. In embodiments, the antibody is administered at an amount from about 360 μg to about 500 μg. In embodiments, the antibody is administered at an amount from about 370 μg to about 500 μg. In embodiments, the antibody is administered at an amount from about 380 μg to about 500 μg. In embodiments, the antibody is administered at an amount from about 390 μg to about 500 μg. In embodiments, the antibody is administered at an amount from about 400 μg to about 500 μg.

In embodiments, the antibody is administered at an amount from about 410 μg to about 500 μg. In embodiments, the antibody is administered at an amount from about 420 μg to about 500 μg. In embodiments, the antibody is administered at an amount from about 430 μg to about 500 μg. In embodiments, the antibody is administered at an amount from about 440 μg to about 500 μg. In embodiments, the antibody is administered at an amount from about 450 μg to about 500 μg. In embodiments, the antibody is administered at an amount from about 460 μg to about 500 μg. In embodiments, the antibody is administered at an amount from about 470 μg to about 500 μg. In embodiments, the antibody is administered at an amount from about 480 μg to about 500 μg. In embodiments, the antibody is administered at an amount from about 490 μg to about 500 μg.

In embodiments, the antibody is administered at an amount from about 10 μg to about 100 μg. In embodiments, the antibody is administered at an amount from about 20 μg to about 100 μg. In embodiments, the antibody is administered at an amount from about 30 μg to about 100 μg. In embodiments, the antibody is administered at an amount from about 40 μg to about 100 μg. In embodiments, the antibody is administered at an amount from about 50 μg to about 100 μg. In embodiments, the antibody is administered at an amount from about 60 μg to about 100 μg. In embodiments, the antibody is administered at an amount from about 70 μg to about 100 μg. In embodiments, the antibody is administered at an amount from about 80 μg to about 100 μg. In embodiments, the antibody is administered at an amount from about 90 μg to about 100 μg.

In embodiments, the antibody is administered at an amount of about 10 μg, 20 μg, 30 g, 40 μg, 50 g, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 210 g, 220 μg, 230 g, 240 g, 250 μg, 260 g, 270 g, 280 μg, 290 μg, 300 μg, 310 μg, 320 μg, 330 g, 340 g, 350 μg, 360 μg, 370 g, 380 μg, 390 μg, 400 μg, 410 μg, 420 μg, 430 μg, 440 μg, 450 μg, 460 μg, 470 μg, 480 μg, 490 μg, or 500 μg.

It is understood that the protein (e.g., the antibody or recombinant protein) provided herein including embodiments thereof may be administered (e.g., as a pharmaceutical composition) at any of the concentrations described herein for the administration of the antibody (e.g., 10 μg-500 μg).

In embodiments, the recombinant protein or antibody can be administered at an amount of about 10 μg-100 μg. In embodiments, the recombinant protein or antibody can be administered at an amount of 10 μg-100 μg. In some embodiments, a pharmaceutical composition comprising the protein (e.g., the antibody or recombinant protein) can be formulated in an aqueous solution at a concentration of 0.200 mg/mL to 2.00 mg/mL, 0.500 mg/mL to 1.50 mg/mL, 0.700 mg/mL to 0.875 mg/mL, or 0.875 mg/mL to 1.25 mg/mL. In some embodiments, a pharmaceutical composition comprising the protein (e.g., the antibody or recombinant protein) can be formulated in an aqueous solution at a concentration of 1.25 mg/mL to 2.50 mg/mL, 2.50 mg/mL to 5.00 mg/mL, 5.00 mg/mL to 10.0 mg/mL, 10.0 mg/mL to 25.0 mg/mL, 25.0 mg/mL to 50.0 mg/mL, 50.0 mg/mL to 100 mg/mL, 100 mg/mL to 150 mg/mL, or 150 mg/mL to 200 mg/mL.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Flow Cytometry Cell Surface Affinity Binding

This example shows cell surface binding of anti-Flt3 antibody compositions described herein.

Table 1 shows in vitro AT19 series asset binding to cell surface FLT3 expressed on MonoMac-6 human monocytes cells. Data are presented as mean fluorescence intensity (MFI) of AlexaFluor 647 fluorophore detected during flow cytometry analysis after incubation of cells with AT19 series antibodies at varying concentrations of antibody (shown in nanomolar (nM) concentration).

TABLE 1

| AT19 Clone | EC50 (nM) |
|---|---|
| AT19.80 | 0.66 |
| AT19.23 | 0.63 |
| AT19.5 | 0.65 |
| AT19.20 | 0.69 |
| AT19.53 | 0.74 |
| AT19.89 | 0.86 |
| AT19.58 | 0.87 |
| AT19.3 | 1.1 |
| AT19.42 | 1.1 |
| AT19.14 | 1.2 |
| AT19.82 | 1.3 |
| AT19.27 | 1.4 |
| AT19.12 | 1.5 |
| AT19.75 | 1.5 |
| AT19.28 | 1.6 |
| AT19.11 | 1.6 |
| AT19.54 | 1.6 |
| AT19.29 | 1.6 |
| AT19.15 | 1.6 |
| AT19.88 | 1.6 |
| AT19.13 | 1.6 |
| AT19.18 | 1.6 |
| AT19.41 | 1.8 |
| AT19.22 | 1.8 |
| AT19.71 | 1.8 |
| AT19.55 | 1.9 |
| AT19.86 | 1.9 |
| AT19.74 | 1.9 |
| AT19.49 | 1.9 |
| AT19.2 | 1.9 |
| AT19.26 | 2.1 |
| AT19.66 | 2.1 |
| AT19.63 | 2.1 |
| AT19.62 | 2.1 |
| AT19.1 | 2.2 |
| AT19.37 | 2.2 |
| AT19.36 | 2.2 |
| AT19.87 | 2.3 |
| AT19.43 | 2.3 |
| AT19.51 | 2.4 |
| AT19.84 | 2.5 |
| AT19.79 | 2.6 |
| AT19.24 | 2.6 |
| AT19.78 | 3.0 |
| AT19.57 | 3.6 |
| AT19.34 | 3.9 |
| AT19.83 | 4.0 |
| AT19.85 | 4.7 |
| AT19.69 | 4.8 |
| AT19.72 | 6.4 |
| AT19.40 | 8.1 |
| AT19.38 | 8.6 |
| AT19.60 | 47 |
| AT19.50 | 78 |
| AT19.21 | 103 |
| AT19.64 | 17744 |
| AT19.61 | 112231 |
| AT19.10 | 174869 |
| AT19.46 | 427569 |

### Example 2: Cytotoxicity Assay

This example shows cytotoxicity testing of anti-FLT3 antibody compositions described herein.

Monomac-6 cells human monocyte cells were blocked with 3% human serum and 5,000 cells/well were added to a white flat bottom 96-well plate. Conditioned hybridoma media was added to cells to a final concentration of 10 nM of the indicated FLT3 antibody clone (“m42” which corresponds to AT-19.42, “m20” which corresponds to AT-19.20, “m89” which corresponds to AT-19.89, “m38” which corresponds to AT-19.38, “m5” which corresponds to AT-19.05, “m23” which corresponds to AT-19.23, “m80” which corresponds to AT-19.80, “m84” which corresponds to AT-19.84, “m61” which corresponds to AT-19.61, “m9” which corresponds to AT-19.09, “m10” which corresponds to AT-19.10, “m79” which corresponds to AT-19.79, “m88” which corresponds to AT-19.88, “m69” which corresponds to AT-19.69, “m57” which corresponds to AT-19.57, “m21” which corresponds to AT-19.21, “m60” which corresponds to AT-19.60, “m37” which corresponds to AT-19.37, “m43” which corresponds to AT-19.43, “m58” which corresponds to AT-19.58, “m27” which corresponds to AT-19.27, “m53” which corresponds to AT-19.53, “m63” which corresponds to AT-19.63, “m55” which corresponds to AT-19.55, “m74” which corresponds to AT-19.74, “m29” which corresponds to AT-19.29, “m50” which corresponds to AT-19.50, “m64” which corresponds to AT-19.64, “m87” which corresponds to AT-19.87, “m85” which corresponds to AT-19.85, “m83” which corresponds to AT-19.83, “m46” which corresponds to AT-19.46, “m22” which corresponds to AT-19.22, “m40” which corresponds to AT-19.40, “m52” which corresponds to AT-19.52, “m09” which corresponds to AT-19.09, “m01” which corresponds to AT-19.01, “m47” which corresponds to AT-19.47, “m28” which corresponds to AT-19.28, “m72” which corresponds to AT-19.72, “m66” which corresponds to AT-19.66, “m75” which corresponds to AT-19.75, “m36” which corresponds to AT-19.36, “m71” which corresponds to AT-19.71, “m34” which corresponds to AT-19.34, “m24” which corresponds to AT-19.24, “m14” which corresponds to AT-19.14, “m15” which corresponds to AT-19.15, “m62” which corresponds to AT-19.62, “m12” which corresponds to AT-19.12, “m54” which corresponds to AT-19.54, “m78” which corresponds to AT-19.78, “m26” which corresponds to AT-19.26, “m82” which corresponds to AT-19.82, “m03” which corresponds to AT-19.03, “m41” which corresponds to AT-19.41, “m18” which corresponds to AT-19.18, “m11” which corresponds to AT-19.11, “m86” which corresponds to AT-19.86, “m13” which corresponds to AT-19.13, “m02” which corresponds to AT-19.02). The toxin conjugated antibody αMFc-CL-DX8951 (Moradec) was added to a final concentration of 20 nM. The plate was incubated in a 37° C. 5% CO₂ incubator for 96 hours. After incubation, cell viability with the CellTiterGlo2 (Promega) reagent as per manufacturers instructions. Data is normalized to a media-only treated cell group (dotted line, indicating 100% of control treatment). Data from these experiments, which are shown in FIG. 1, illustrate that several AT-19 family antibodies (including AT-19.20, AT-19.38, and AT-19.84) substantially reduce cell viability of MonoMac-6 cells when bound to anti-mouse Fc secondary antibodies conjugated to DX8951 topoisomerase inhibitor via a CL linker. In particular, AT-19.20, AT-19.38, and AT-19.84 anti-FLT3 antibodies each result in a decrease of Monomac-6 cell viability of over 75%, compared to media-only controls, when the anti-FLT3 antibody is bound to a topoisomerase inhibitor via a secondary antibody.

### Example 3: Binding of Chimeric Antibodies to Cells Expressing FLT3 or to Cynomolgus FLT3

This example shows binding of anti-FLT3 antibody compositions described herein to HEK293 cells showing surface expression of human FLT3 protein or to immobilized cynomolgus FLT3 protein in vitro.

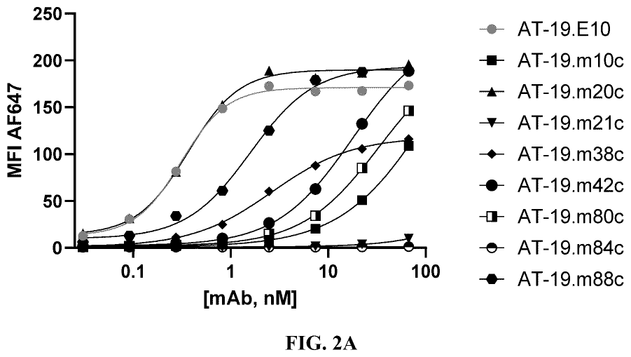

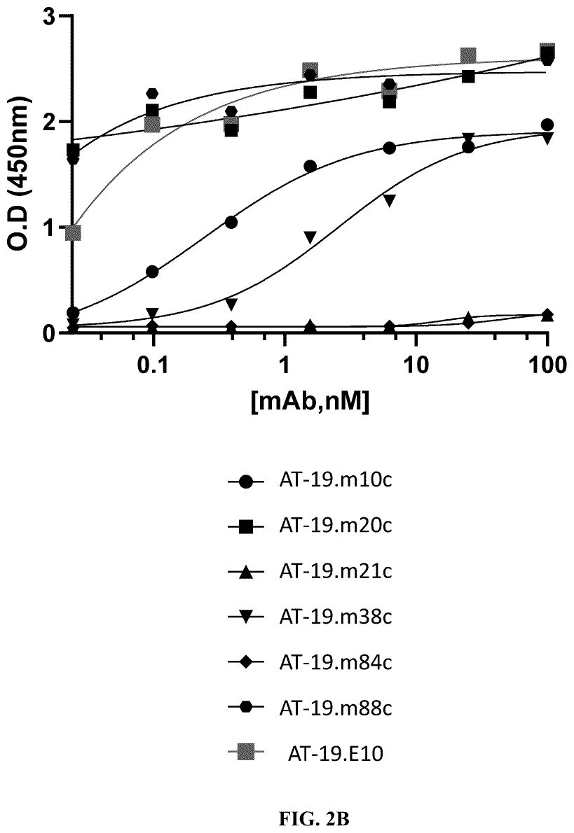

FIG. 2A shows binding of chimeric AT-19 anti-FLT3 antibody clones to human HEK293 cells expressing human FLT3 protein, which is expressed on leukemias such as acute myeloid leukemia (AML) and acute lymphoblastic leukemia (ALL). The collected data show that chimeric AT-19 family antibodies AT19.20c (“AT-19.M20c”, small upward triangles; calculated EC50 value: 0.36 nanomolar (“nM”)), AT19.38c (“AT-19.m38c”, green diamonds; calculated EC50 value: 2.7 nM), and AT19.88c (“AT-19.m88c”, large downward triangles; calculated EC50 value: 1.6 nM) all showed positive binding to human FLT3-expressing HEK293 cells. Chimeric AT19 family clones AT19.42c (“AT-19.m42c”, green circles; calculated EC50 value: 18 nM), AT19.80c (“AT-19.m80c”, large green squares; calculated EC50 value: 33 nM), AT19.84c (“AT-19.m84c”, large upward triangles; calculated EC50 value: 51 nM), and AT19.10c (“AT-19.m10c”, small green squares; calculated EC50 value: 118 nM) showed binding to human FLT3-expressing HEK293 cells to a lesser degree. Chimeric AT19.21c antibody (“AT-19.m21c”, small downward triangles; no calculated EC50 value) did not show strong binding to human FLT3-expressing HEK 293 cells. Anti-FLT3 antibody AT-19 E10 showed positive binding to human FLT3-expressing HEK 293 cells (“AT-19.E10”, black circles; calculated EC50 value: 0.3 nM). In all, the data show that chimeric AT-19 family antibodies bind to cells expressing human FLT3 protein.

FIG. 2B shows binding of chimeric AT-19 anti-FLT3 antibody clones to immobilized cynomolgus FLT3 protein, as detected by enzyme-linked immunosorbent assay (ELISA). Binding was observed for AT19.10c antibody (“AT-19.m10c”, small green circles; calculated EC50 value: 0.25 nM), AT19.21c antibody (“AT-19.m21c”, upward triangles; calculated EC50 value: 16 nM), AT19.38c (“AT-19.m38c”, downward triangles; calculated EC50 value: 2.5 nM), and, to a lesser degree, AT19.84c (“AT-19.m84c”, green diamonds; calculated EC50 value: 48 nM). No EC50 value was calculated from the data for AT19.20c (“AT-19.M20c”, green squares), AT19.88c (“AT-19.M88c”, large green circles), or AT-19 E10 antibodies (“AT-19.E10”, black squares).

### Example 4: Cytotoxicity Assay

This example shows cytotoxicity testing of anti-FLT3 antibody compositions described herein.

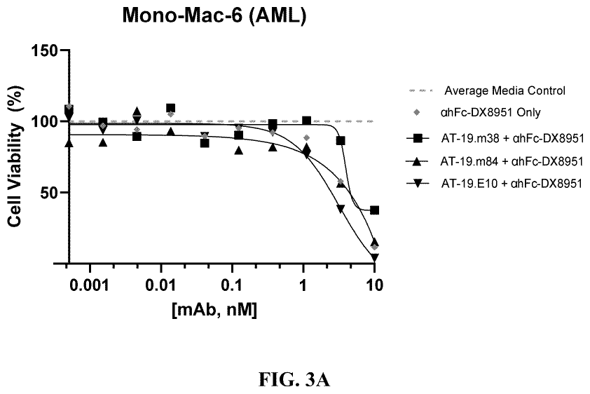

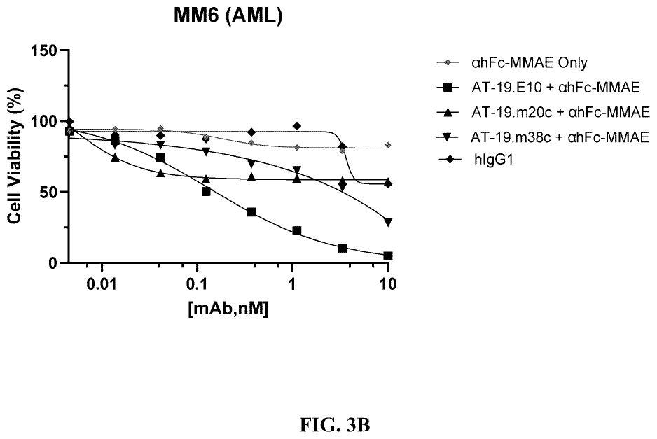

MonoMac-6 (“MM6”) cells human monocyte cells centrifuged and resuspended at a concentration of 62,500 cells/mL in MM6 culture medium. 80 microliters (μL) of the resuspended cell sample were added to the wells of a white 96-well plate to achieve an initial plating density of 50,000 cells/well. A toxic payload-conjugated secondary antibody (antibody anti-mouse-Fc-CL-DX8951 (Moradec)) prepared in MM6 buffer to a concentration of 100 nM. Primary anti-FLT3 antibodies (AT-19.38, AT-19.84, and AT-19 E10) were prepared individually in MM6 medium to and added to the secondary antibody solution to achieve a final primary antibody concentration of 50 nM. The solutions containing the primary and secondary antibody were diluted using serial 1:3 dilutions, and 20 μL of the solution containing the primary antibody and the secondary antibody were added to the cells. The plated MonoMac-6 cells were incubated with the solution containing the primary and secondary antibody in a 37° C., 5% CO₂ incubator for 6 days. An additional group of cultured MonoMac-6 cells were sham-treated with media vehicle only for 6 days. After incubation, cell viability with the CellTiterGlo2 (Promega) reagent as per the manufacturer's instructions. Data is shown in FIG. 3A, and calculated IC50 values and maximum cell killing % are as listed: (AT19.m38, 3.9 nM; AT19.m84, not calculated; AT19.E10, 3.2 nM) (AT19.m38, 62%; AT19.m84, 85%; AT19.E10, 96%). Cell viability data was normalized to the media-only treated cell group (dotted line in FIG. 3A). Data shows that AT-19.38 antibodies were similarly effective at reducing Monomac-6 cell viability when bound to a secondary antibody conjugated to DX8951 (“AT-19.m38+αhFc-DX8951”; IC50 value of 3.9) as AT-19 E10 antibody (which is also referenced herein as “ALT-Anti-Flt3” or “AT-19.E10+αhFc8951” and comprises amino acid sequences reflected in SEQ ID NOs: 193 and 194 and nucleic acid sequences reflected in SEQ ID NOs: 195 and 196)) bound to a secondary antibody conjugated to DX8951 (IC50 value of 3.2). Therefore, anti-FLT3 antibodies described herein (e.g., AT-19 family antibodies described herein) are effective at delivering a toxic payload to monocytic cells of a population and thereby reducing the viability of the monocytic cell population.

FIG. 3B shows in vitro cytotoxicity testing of AT-19 antibodies and AT-19 E10 antibody bound to a secondary antibody linked to a monomethyl auristatin E (MMAE) payload (anti-human-Fc-CL-MMAE drug-conjugated secondary antibody purchased from Moradec LLC). Chimeric AT-19.20c anti-FLT3 antibody bound to MMAE-conjugated secondary antibody (“AT-19.M20c+αhFc-MMAE”; upward triangles) showed an IC50 value of 0.004 nanomolar (nM) and maximum cell killing of 43%. AT-19 E10 anti-FLT3 antibody bound to MMAE-conjugated secondary antibody (“AT-19.E10+αhFc-MMAE”; squares) was also effective at reducing viability of MM6 cells, with a calculated IC50 value of 0.14 nM and maximum cell killing of 95%. AT-19.38c antibody (“AT-19.m38c+αhFc-MMAE”; downward triangles), MMAE-coupled secondary antibody alone (“+αhFc-MMAE Only”), and human IgG1 alone (“hIgG1”) treatments did not appear to be as effective in reducing MM6 cell viability.

### Example 5: Affinity Binding Assay

This example shows high affinity binding testing of anti-FLT3 antibody compositions described herein.

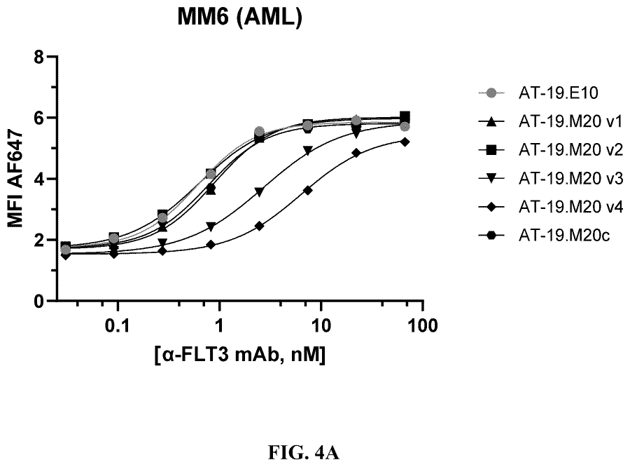

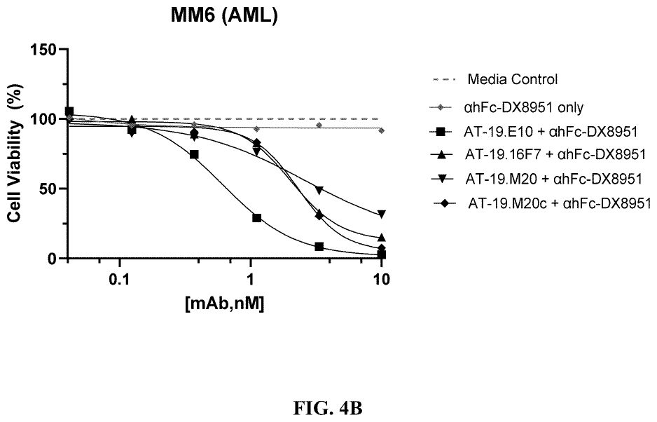

Data presented in FIG. 4A for AT-19 E10 antibody (“AT-19. E10”; black circles; EC50 value: 0.6 nM), humanized AT-19.20 h.v1 antibody (“AT-19. M20 h v1”; upward triangles; EC50 value: 0.9 nM), humanized AT-19.20 h.v2 antibody (“AT-19. M20 h v2”; downward triangles; EC50 value: 0.6 nM), humanized AT-19.20 h.v3 antibody (“AT-19. M20 h v3”; diamonds; EC50 value: 2.70 nM), humanized AT-19.20 h.v4 antibody (“AT-19. M20 h v4”; circles; EC50 value: 6.4 nM) and chimeric AT-19.20c antibody (“AT-19. M20c”; squares; EC50 value: 0.8 nM) showed positive binding of humanized and chimeric AT-19 family antibodies, including AT-19 E10 antibodies.

FIG. 4B shows effects of AT-19 anti-FLT3 antibodies disclosed herein and bound to DX8951-conjugated secondary antibodies on MM6 AML cells. IC50 values for viability of MM6 cells after treatment with humanized AT19.20 h antibody bound to DX8951-conjugated secondary antibody (“AT-19.M20+αhFc-DX8951”; diamonds; IC50 value: 2.6 nM; maximum cell killing: 69%), chimeric AT19.20c antibody bound to DX8951-conjugated secondary antibody (“AT-19.m20c+αhFc-DX8951”; circles; IC50 value: 2.3 nM; maximum cell killing: 93%), 16F7 AT19-family antibody bound to DX8951-conjugated secondary antibody (“AT-19.16710+αhFc-DX8951”; downward triangles; IC50 value: 2 nM; maximum cell killing: 85%), and AT-19 E10 anti-FLT3 antibody bound to DX8951-conjugated secondary antibody (“AT-19.E10+αhFc-DX8951”; upward triangles; IC50 value: 0.6120 nM; maximum cell killing: 97%) all showed effective reduction of viability in targeted MM6 AML cells (see FIG. 4B and FIG. 4C). In contrast, media only (circles) and DX8951-conjugated secondary antibody only controls (“αhFc-DX8951 only”, squares) did not effectively reduce viability of treated MM6 cells.

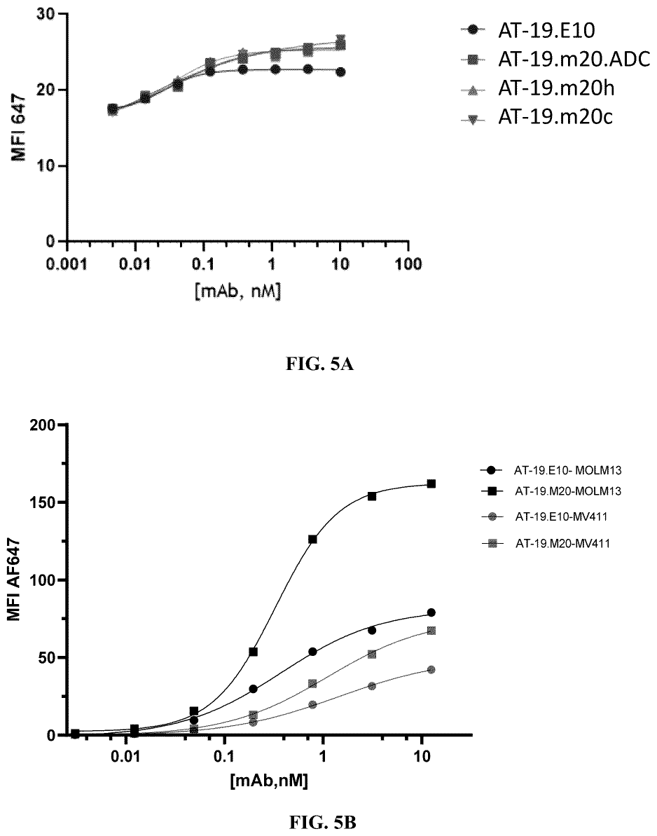

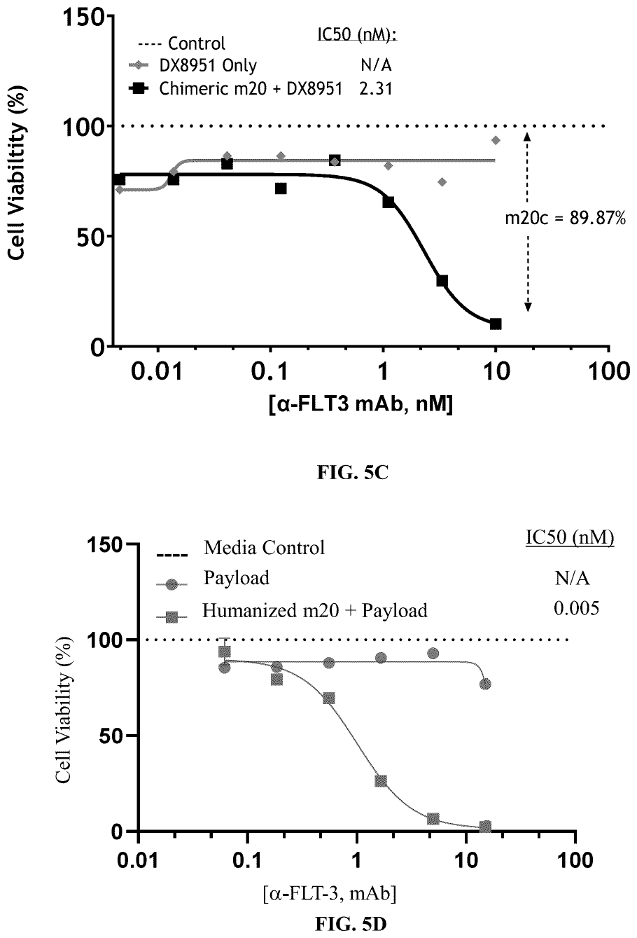

Affinity binding of humanized AT-19.20 h antibodies (“AT-19.M20”; upward green triangles), chimeric AT-19.20c antibodies (“AT-19.M20c”; downward purple triangles), and AT-19.20 antibody conjugated to DXd via GGFG linker (“AT-19.M20.ADC”; red squares), and AT-19 E10 antibodies (“AT-19.E10”, blue circles) to MM6 AML cells was performed by incubating antibodies with MM6 cells at various concentrations (x-axis values indicate nanomolar (nM) concentration of antibody) and detected by fluorescent imaging of AF647 fluorophores conjugated to the secondary antibody (FIG. 5A). All tested groups showed good binding to MM6 cells, even when a payload is conjugated to the anti-FLT3 AT-19 family antibody, with EC50 values for “E10”, “M20ADC”, “M20”, and “M20c” experimental groups calculated to be 0.02 nanomolar (nM), 0.04 nM, 0.03 nM, and 0.01 nM. As shown in FIG. 5B, AT19.M20 and AT19.E10 exhibit strong binding to acute myelogenous leukemia cell lines with the following EC50 values: AT19.M20: MV4-11, 2.7 nM and MOLM-13, 0.38 nM; and AT 19.E10: MV4-11, 3.3 nM and MOLM-13, 9.1 nM.

### Example 6: Acute Myeloid Leukemia and Acute Lymphoblastic Leukemia Cytotoxicity Assays

This example shows the effects of anti-FLT3 antibody-drug conjugate compositions on acute myeloid leukemia (AML) and acute lymphoblastic leukemia (ALL) cells.

FIG. 5C shows the effects of AT-19.20 chimeric antibody bound to a secondary antibody conjugated to DXd payload (squares) on MM6 acute myeloid leukemia (AML) cell viability compared to payload-only treatment (circles) and media-only control, which the results are normalized to (dotted line). The chimeric AT-19.20 antibody with payload was found to have an IC50 of 2.3 nM, and 90% reduction in viability of AML cells compared to media-only treated cells.

FIG. 5D shows the effects of AT-19.20 humanized antibody bound to a secondary antibody conjugated to DXd payload (squares) on MM6 acute myeloid leukemia (AML) cell viability compared to payload-only treatment (circles) and media-only control, which the results are normalized to (dotted line). The humanized AT-19.20 antibody with payload was found to have an IC50 of 0.005 nM, and 98% reduction in viability of AML cells compared to media-only treated cells.

In related experiments, chimeric AT-19 family antibodies conjugated directly to DXd via a GGFG linker were effective in reducing the viability of colorectal cancer cells, gastric cancer cells, and pancreatic cells in separate cytotoxicity assays, with chimeric AT-19 antibodies conjugated to DXd reducing colorectal cancer cell viability by up to 88% versus media only control treatment (data not shown).

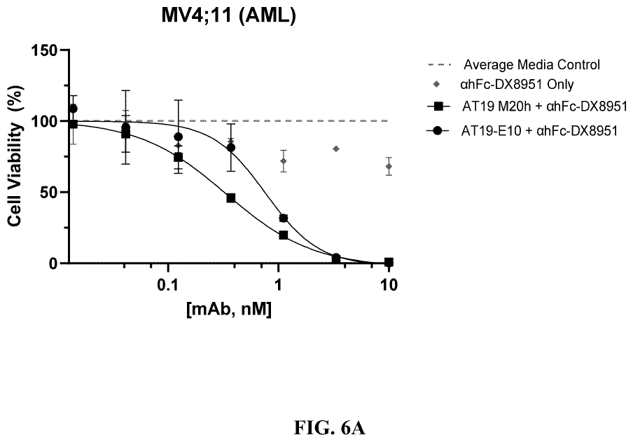

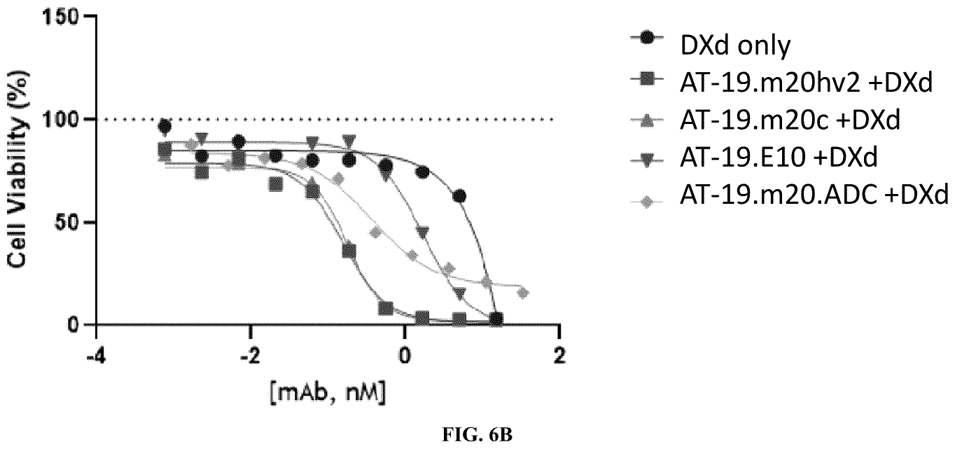

FIG. 6A shows the effect of humanized AT19.20 h anti-FLT3 antibody bound to an anti-human Fc secondary antibody conjugated to DX8951 payload (“AT-19 M20h+αhFc-DX8951”; squares) on MV4-11 acute myeloid leukemia (AML) cell viability compared to AT-19 E10 anti-FLT3 antibody bound to the anti-human Fc secondary antibody conjugated to DX8951 payload (“AT-19.E10+αhFc-DX8951”; circles), and anti-human Fc secondary antibody with DX8951 only control (“αHFc-DX8951 Only”; diamonds). Results showed that AT19.m20 h antibody bound to DX8951-conjugated secondary and AT-19 E10 antibody bound to DX8951-conjugated secondary antibody bound to DX8951 effectively reduced MV4-11 cell viability, with calculated IC50 values of 0.341 nanomolar (nM) and 0.77 nM, respectively. The maximum cell killing was 99% for AT19.M20 h and 100% for AT19.E10. All data was normalized against media control treatments (dotted line).

FIG. 6B shows effects of AT19.20 family anti-FLT3 antibodies on MV4-11 AML cell viability. Humanized AT19.M20 anti-FLT3 antibody directly conjugated to DXd payload via a GGFG linker (“AT-19.M20.ADC+DXd”, orange diamonds) produced an IC50 value of 0.37 nM. Humanized AT19.20 hv.2 anti-FLT3 antibody conjugated to DXd (“AT-19.m20hv2+DXd”, red squares) produced an IC50 value of 0.15 nM, whereas chimeric AT19.20c anti-FLT3 antibody conjugated to DXd (“AT-19.m20c+DXd”, green upward triangles) produced an IC50 value of 0.18 nM. AT-19 E10 anti-FLT3 antibody conjugated to DXd (“AT-19.E10+DXd”, purple downward triangles) produced an IC50 value of 1.68 nM. Therefore, humanized and chimeric AT19 family antibodies are effective at reducing the viability of AML cells, e.g., when conjugated or bound to an antineoplastic payload.

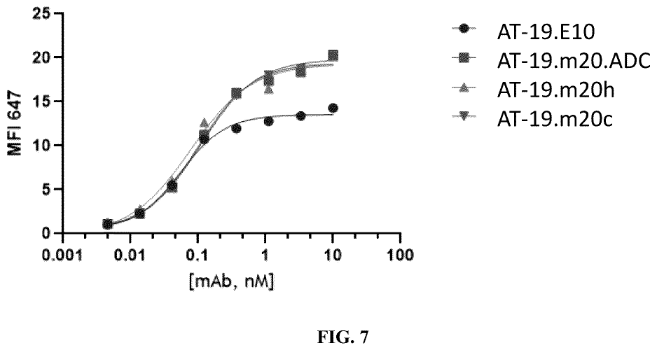

FIG. 7 shows binding of AT-19 family anti-FLT3 antibodies to SEM acute lymphoblastic leukemia (ALL) cells. Chimeric AT19.20c (“AT-19.M20c”) antibodies, humanized AT19.20h (“AT-19.M20 h”) antibodies, and humanized AT19.20 h antibodies bound directly to DXd payload via a GGFG linker (“AT-19.M20ADC”) all showed strong binding to SEM cells, with EC50 values of 0.11 nanomolar (nM), 0.08 nM, and 0.1 nM calculated for the tested AT19.20 family antibody variants, respectively. AT-19 E10 anti-FLT3 antibody also effectively bound to SEM cells, as shown in FIG. 7, with a calculated EC50 value of 0.06 nM. These data show that chimeric and human variants of AT-19 family antibodies bind well to SEM cells, even when the AT-19 family antibody is conjugated to an antineoplastic payload.

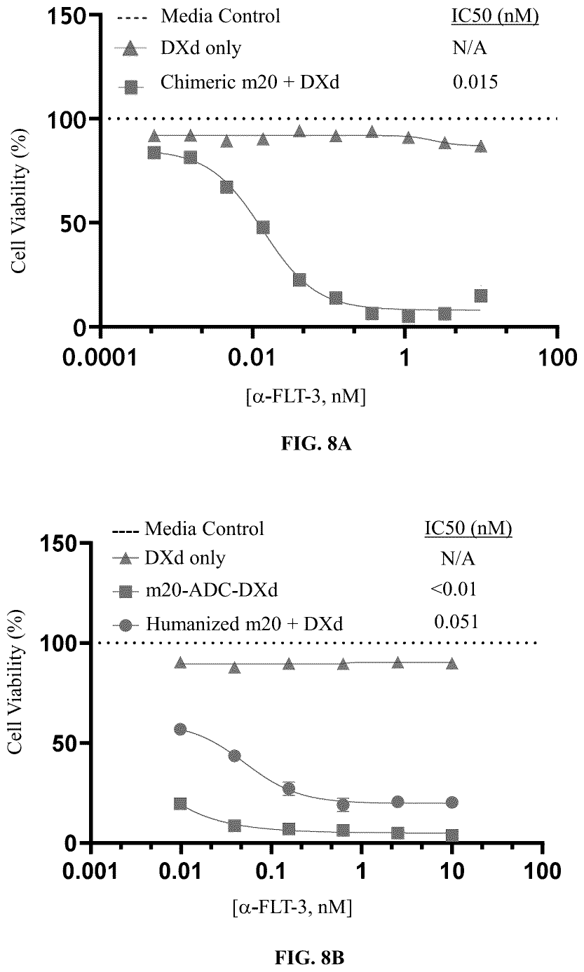

FIG. 8A shows the effects of AT-19.20 chimeric antibody bound to a secondary antibody conjugated to DXd payload (squares) on SEM acute lymphoblastic leukemia (ALL) cell viability compared to payload-only treatment (triangles) and media-only control, which the results are normalized to (dotted line). The chimeric AT-19.20 antibody with payload was found to have an IC50 of 0.015 nM, and 87% reduction in viability of ALL cells compared to media-only treated cells.

FIG. 8B shows the effects of AT-19.20 humanized antibody bound to a secondary antibody conjugated to DXd payload (circles) on SEM acute lymphoblastic leukemia (ALL) cell viability compared to payload-only treatment (triangles) and media-only control, which the results are normalized to (dotted line). The humanized AT-19.20 antibody with payload was found to have an IC50 of 0.051 nM, and 95% reduction in viability of ALL cells compared to media-only treated cells. Humanized AT-19.20 antibodies with directly conjugated DXd reduced viability of ALL cells (IC50 of less than 0.01 nM) compared to payload-only and media-only controls.

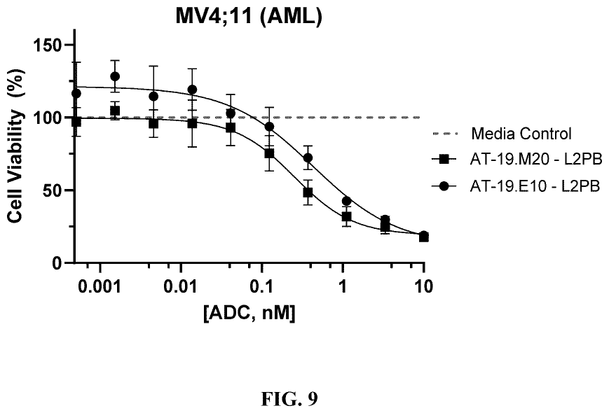

FIG. 9 shows the effects of AT-19.M20 (black squares) and AT-19.E10 (black circles) humanized antibodies directly conjugated to the MMAE (L2PB: L2, VC-PAB; PB, MMAE) payload on MV4-11 acute myelogenous leukemia (AML) cell viability compared to the media-only control, which the results are normalized to (dotted line). The humanized AT-19.M20 antibody with payload was found to have an IC50 of 0.2 nM, and 82% reduction in viability of AML cells compared to media-only treated cells. The humanized AT-19.E10 antibody with payload was found to have an IC50 of 0.4 nM, and 81% reduction in viability of AML cells compared to media-only treated cells.

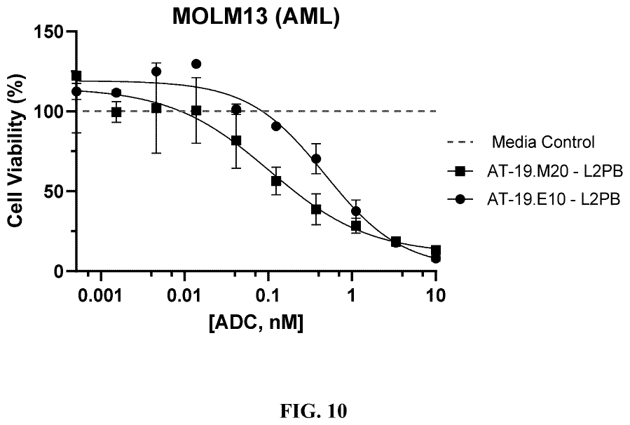

FIG. 10 shows the effects of AT-19.M20 (black squares) and AT-19.E10 (black circles) humanized antibodies directly conjugated to the MMAE (L2PB: L2, VC-PAB; PB, MMAE) payload on MOLM-13 acute myelogenous leukemia (AML) cell viability compared to the media-only control, which the results are normalized to (dotted line). The humanized AT-19.M20 antibody with payload was found to have an IC50 of 0.1 nM, and 87% reduction in viability of AML cells compared to media-only treated cells. The humanized AT-19.E10 antibody with payload was found to have an IC50 of 0.5 nM, and 92% reduction in viability of AML cells compared to media-only treated cells.

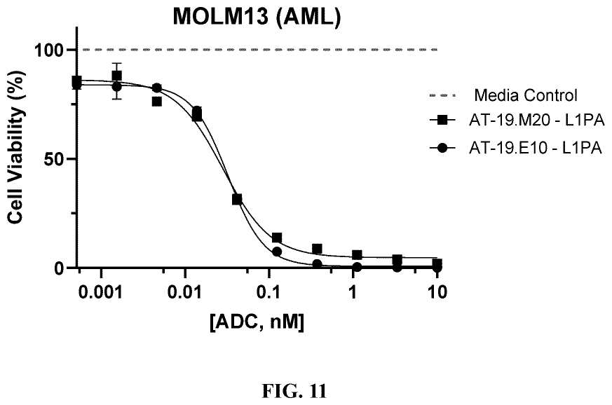

FIG. 11 shows the effects of AT-19.M20 (black squares) and AT-19.E10 (black circles) humanized antibodies directly conjugated to the DXd (L1PA: L1, GGFG; PA, DXd) payload on MOLM-13 acute myelogenous leukemia (AML) cell viability compared to the media-only control, which the results are normalized to (dotted line). The humanized AT-19.M20 antibody with payload was found to have an IC50 of 0.03 nM, and 98% reduction in viability of AML cells compared to media-only treated cells. The humanized AT-19.E10 antibody with payload was found to have an IC50 of 0.03 nM, and 100% reduction in viability of AML cells compared to media-only treated cells.

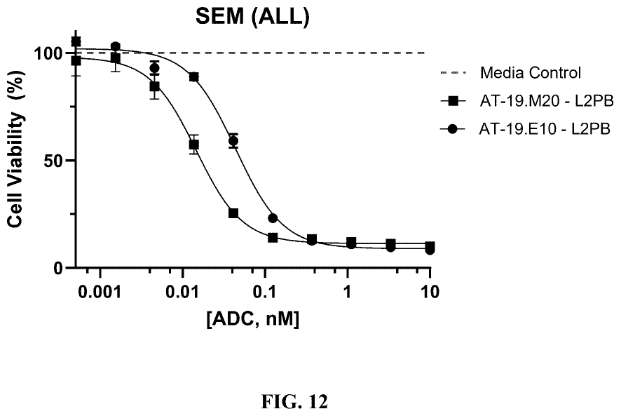

FIG. 12 shows the effects of AT-19.M20 (black squares) and AT-19.E10 (black circles) humanized antibodies directly conjugated to the MMAE (L2PB: L2, VC-PAB; PB, MMAE) payload on SEM acute lymphoblastic leukemia (ALL) cell viability compared to the media-only control, which the results are normalized to (dotted line). The humanized AT-19.M20 antibody with payload was found to have an IC50 of 0.01 nM, and 90% reduction in viability of ALL cells compared to media-only treated cells. The humanized AT-19.E10 antibody with payload was found to have an IC50 of 0.04 nM, and 92% reduction in viability of ALL cells compared to media-only treated cells.

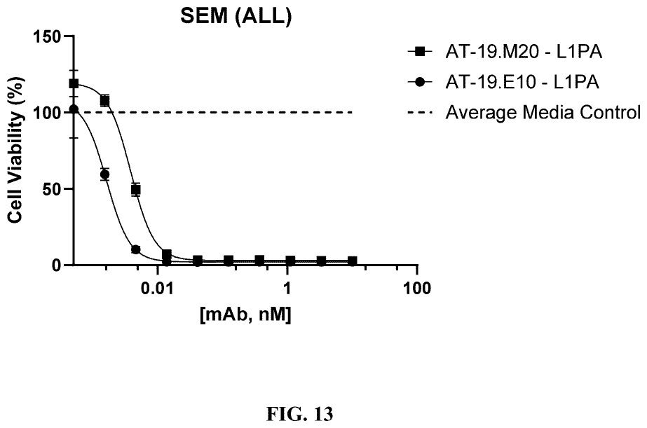

FIG. 13 shows the effects of AT-19.M20 (black squares) and AT-19.E10 (black circles) humanized antibodies directly conjugated to the DXd (L1PA: L1, GGFG; PA, DXd) payload on SEM acute lymphoblastic leukemia (ALL) cell viability compared to the media-only control, which the results are normalized to (dotted line). The humanized AT-19.M20 antibody with payload was found to have an IC50 of 0.01 nM, and 97% reduction in viability of ALL cells compared to media-only treated cells. The humanized AT-19.E10 antibody with payload was found to have an IC50 of 0.04 nM, and 98% reduction in viability of ALL cells compared to media-only treated cells.

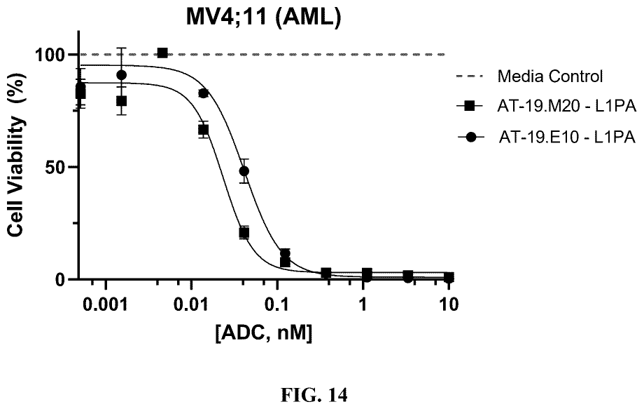

FIG. 14 shows the effects of AT-19.M20 (black squares) and AT-19.E10 (black circles) humanized antibodies directly conjugated to the DXd (L1PA: L1, GGFG; PA, DXd) payload on MV-4-11 acute myelogenous leukemia (AML) cell viability compared to the media-only control, which the results are normalized to (dotted line). The humanized AT-19.M20 antibody with payload was found to have an IC50 of 0.02 nM, and 99% reduction in viability of ALL cells compared to media-only treated cells. The humanized AT-19.E10 antibody with payload was found to have an IC50 of 0.04 nM, and 100% reduction in viability of ALL cells compared to media-only treated cells.

### TABLES

TABLE 2
Sequences according to embodiments of AT-19 Assets (e.g., anti-FLT3 antibodies)

| SEQ ID | Asset | Type | Chain | Region | Sequence |
|---|---|---|---|---|---|
| 1 | AT- | protein | Light | CDR1 | KSSQSLLNSGNQKNYLT |
| | 19.10 | | | | |
| 2 | AT- | protein | Light | CDR2 | WAATRES |
| | 19.10 | | | | |
| 3 | AT- | protein | Light | CDR3 | QNDYTYPLT |
| | 19.10 | | | | |
| 4 | AT- | protein | Heavy | CDR1 | NYWIE |
| | 19.10 | | | | |
| 5 | AT- | protein | Heavy | CDR2 | EIFPGSGTTKYNEKFKG |
| | 19.10 | | | | |
| 6 | AT- | protein | Heavy | CDR3 | GRFISTTRGFAY |
| | 19.10 | | | | |
| 7 | AT- | protein | Light | Variable | DIVMTQSPSSLTVTPGEKVTMSC |
| | 19.10 | | | | KSSQSLLNSGNQKNYLTWYQQK |
| | | | | | PGQPPKLLIYWAATRESGVPDRF |
| | | | | | TGSGSGTDFTLTISSVQAEDLAV |
| | | | | | YYCQNDYTYPLTFGAGTKLELK |
| 8 | AT- | protein | Heavy | Variable | QVQLQQSGAELTKPGASVKISCK |
| | 19.10 | | | | ATGYTFSNYWIEWVIQRPGHGLE |
| | | | | | WIGEIFPGSGTTKYNEKFKGKAT |
| | | | | | FTADTSSNTAYMQLNSLTSEDSA |
| | | | | | VYYCARGRFISTTRGFAYWGQG |
| | | | | | TLVTVSA |
| 9 | AT- | protein | Light | CDR1 | KASQDVSTAVA |
| | 19.20 | | | | |
| 10 | AT- | protein | Light | CDR2 | SASYRYT |
| | 19.20 | | | | |
| 11 | AT- | protein | Light | CDR3 | QQHFNTPRT |
| | 19.20 | | | | |
| 12 | AT- | protein | Heavy | CDR1 | SYAMS |
| | 19.20 | | | | |
| 13 | AT- | protein | Heavy | CDR2 | SINSGGFTYYPDSVKGR |
| | 19.20 | | | | |
| 14 | AT- | protein | Heavy | CDR3 | GGTTVFDY |
| | 19.20 | | | | |
| 15 | AT- | protein | Light | Variable | DIVMTQSHKFMSTSVGDRVSITC |
| | 19.20 | | | | KASQDVSTAVAWYQQKPGHSPK |
| | | | | | LLIYSASYRYTGVPDRFTGSGSG |
| | | | | | ADFTFTISSVQAEDLAVYFCQQH |
| | | | | | FNTPRTFGGGTKLEIK |
| 16 | AT- | protein | Heavy | Variable | EVKLVESGGGLVKPGGSLKLSCA |
| | 19.20 | | | | ASGFTFSSYAMSWVRQTPEKRLE |
| | | | | | WVASINSGGFTYYPDSVKGRFTI |
| | | | | | SRDNTRNILYLQLSSLRSEDTAM |
| | | | | | YYCARGGTTVFDYWGQGTTLTV |
| | | | | | SS |
| 17 | AT- | protein | Light | CDR1 | KASQDVSTAVA |
| | 19.20h. | | | | |
| | v1 | | | | |
| 18 | AT- | protein | Light | CDR2 | SASYRYT |
| | 19.20h. | | | | |
| | v1 | | | | |
| 19 | AT- | protein | Light | CDR3 | QQHFNTPRT |
| | 19.20h. | | | | |
| | v1 | | | | |
| 20 | AT- | protein | Heavy | CDR1 | SYAMS |
| | 19.20h. | | | | |
| | v1 | | | | |
| 21 | AT- | protein | Heavy | CDR2 | SINSGGFTYYPDSVKG |
| | 19.20h. | | | | |
| | v1 | | | | |
| 22 | AT- | protein | Heavy | CDR3 | GGTTVFDY |
| | 19.20h. | | | | |
| | v1 | | | | |
| 23 | AT- | protein | Light | Variable | DIQMTQSPSSLSASVGDRVTITCK |
| | 19.20h. | | | | ASQDVSTAVAWYQQKPGKAPKL |
| | v1 | | | | LIYSASYRYTGVPSRFSGSGSGA |
| | | | | | DFTFTISSLQPEDIATYYCQQHFN |
| | | | | | TPRTFGGGTKVEIK |
| 24 | AT- | protein | Heavy | Variable | EVQLVESGGGLVQPGGSLRLSCA |
| | 19.20h. | | | | ASGFTFSSYAMSWVRQAPGKGL |
| | v1 | | | | EWVASINSGGFTYYPDSVKGRFT |
| | | | | | ISRDNSKNTLYLQMNSLRAEDTA |
| | | | | | VYYCARGGTTVFDYWGQGTLV |
| | | | | | TVSS |
| 25 | AT- | protein | Light | CDR1 | KASQDVSTAVA |
| | 19.20h. | | | | |
| | v2 | | | | |
| 26 | AT- | protein | Light | CDR2 | SASYRYT |
| | 19.20h. | | | | |
| | v2 | | | | |
| 27 | AT- | protein | Light | CDR3 | QQHFNTPRT |
| | 19.20h. | | | | |
| | v2 | | | | |
| 28 | AT- | protein | Heavy | CDR1 | SYAMS |
| | 19.20h. | | | | |
| | v2 | | | | |
| 29 | AT- | protein | Heavy | CDR2 | SINSGGFTYYPQSVQG |
| | 19.20h. | | | | |
| | v2 | | | | |
| 30 | AT- | protein | Heavy | CDR3 | GGTTVFDY |
| | 19.20h. | | | | |
| | v2 | | | | |
| 31 | AT- | protein | Light | Variable | DIQMTQSPSSLSASVGDRVTITCK |
| | 19.20h. | | | | ASQDVSTAVAWYQQKPGKAPKL |
| | v2 | | | | LIYSASYRYTGVPSRFSGSGSGA |
| | | | | | DFTFTISSLQPEDIATYYCQQHFN |
| | | | | | TPRTFGGGTKVEIK |
| 32 | AT- | protein | Heavy | Variable | QVKLVQSGAEVKKPGASLKVSC |
| | 19.20h. | | | | AASGFTFSSYAMSWVRQTPEQR |
| | v2 | | | | LEWVASINSGGFTYYPQSVQGRF |
| | | | | | TISRDNTRNILYLELSSLRSEDTA |
| | | | | | MYYCARGGTTVFDYWGQGTTL |
| | | | | | TVSS |
| 33 | AT- | protein | Light | CDR1 | RASQDVSTAVN |
| | 19.20h. | | | | |
| | v3 | | | | |
| 34 | AT- | protein | Light | CDR2 | SASYLYT |
| | 19.20h. | | | | |
| | v3 | | | | |
| 35 | AT- | protein | Light | CDR3 | QQHFNTPRT |
| | 19.20h. | | | | |
| | v3 | | | | |
| 36 | AT- | protein | Heavy | CDR1 | SYAMS |
| | 19.20h. | | | | |
| | v3 | | | | |
| 37 | AT- | protein | Heavy | CDR2 | SINSGGFTYYPDSVKG |
| | 19.20h. | | | | |
| | v3 | | | | |
| 38 | AT- | protein | Heavy | CDR3 | GGTTVFDY |
| | 19.20h. | | | | |
| | v3 | | | | |
| 39 | AT- | protein | Light | Variable | DIQMTQSHSFMSASVGDRVSITC |
| | 19.20h. | | | | RASQDVSTAVNWYQQKPGHAP |
| | v3 | | | | KLLIYSASYLYTGVPDRFTGSGS |
| | | | | | GADFTFTISSLQAEDLAVYFCQQ |
| | | | | | HFNTPRTFGGGTKLEIK |
| 40 | AT- | protein | Heavy | Variable | EVQLVESGGGLVQPGGSLRLSCA |
| | 19.20h. | | | | ASGFTFSSYAMSWVRQAPGKGL |
| | v3 | | | | EWVASINSGGFTYYPDSVKGRFT |
| | | | | | ISRDNSKNTLYLQMNSLRAEDTA |
| | | | | | VYYCARGGTTVFDYWGQGTLV |
| | | | | | TVSS |
| 41 | AT- | protein | Light | CDR1 | RASQDVSTAVN |
| | 19.20h. | | | | |
| | v4 | | | | |
| 42 | AT- | protein | Light | CDR2 | SASYLYT |
| | 19.20h. | | | | |
| | v4 | | | | |
| 43 | AT- | protein | Light | CDR3 | QQHFNTPRT |
| | 19.20h. | | | | |
| | v4 | | | | |
| 44 | AT- | protein | Heavy | CDR1 | SYAMS |
| | 19.20h. | | | | |
| | v4 | | | | |
| 45 | AT- | protein | Heavy | CDR2 | SINSGGFTYYPQSVQG |
| | 19.20h. | | | | |
| | v4 | | | | |
| 46 | AT- | protein | Heavy | CDR3 | GGTTVFDY |
| | 19.20h. | | | | |
| | v4 | | | | |
| 47 | AT- | protein | Light | Variable | DIQMTQSHSFMSASVGDRVSITC |
| | 19.20h. | | | | RASQDVSTAVNWYQQKPGHAP |
| | v4 | | | | KLLIYSASYLYTGVPDRFTGSGS |
| | | | | | GADFTFTISSLQAEDLAVYFCQQ |
| | | | | | HFNTPRTFGGGTKLEIK |
| 48 | AT- | protein | Heavy | Variable | QVKLVQSGAEVKKPGASLKVSC |
| | 19.20h. | | | | AASGFTFSSYAMSWVRQTPEQR |
| | v4 | | | | LEWVASINSGGFTYYPQSVQGRF |
| | | | | | TISRDNTRNILYLELSSLRSEDTA |
| | | | | | MYYCARGGTTVFDYWGQGTTL |
| | | | | | TVSS |
| 49 | AT- | protein | Light | CDR1 | KASQNVGTNVA |
| | 19.21 | | | | |
| 50 | AT- | protein | Light | CDR2 | SASYRYS |
| | 19.21 | | | | |
| 51 | AT- | protein | Light | CDR3 | QQYNSYPLT |
| | 19.21 | | | | |
| 52 | AT- | protein | Heavy | CDR1 | SYAMS |
| | 19.21 | | | | |
| 53 | AT- | protein | Heavy | CDR2 | TISSGGSYTYYLDSVKG |
| | 19.21 | | | | |
| 54 | AT- | protein | Heavy | CDR3 | PPARAGIYYYVMDY |
| | 19.21 | | | | |
| 55 | AT- | protein | Light | Variable | DIVMTQSQKFMSTSVGDRVSVT |
| | 19.21 | | | | CKASQNVGTNVAWYQQKPGQS |
| | | | | | PKPLIYSASYRYSGVPDRFTGSGS |
| | | | | | GTDFTLTISNVQSEDLAEYFCQQ |
| | | | | | YNSYPLTFGAGTKLELK |
| 56 | AT- | protein | Heavy | Variable | EVQLVESGGGLVKPGGSLKLSCA |
| | 19.21 | | | | ASGFTFSSYAMSWVRQTPEKRLE |
| | | | | | WVATISSGGSYTYYLDSVKGRFT |
| | | | | | ISRDNAKNTLYLQMSSLRSEDTA |
| | | | | | MYYCARPPARAGIYYYVMDYW |
| | | | | | GQGTSVTVSS |
| 57 | AT- | protein | Light | CDR1 | RASQDINNYLH |
| | 19.38 | | | | |
| 58 | AT- | protein | Light | CDR2 | YTSRLHS |
| | 19.38 | | | | |
| 59 | AT- | protein | Light | CDR3 | QQGKTLPWT |
| | 19.38 | | | | |
| 60 | AT- | protein | Heavy | CDR1 | DYGMH |
| | 19.38 | | | | |
| 61 | AT- | protein | Heavy | CDR2 | YISTGSGTIYYADTVKG |
| | 19.38 | | | | |
| 62 | AT- | protein | Heavy | CDR3 | QGYSYTMDY |
| | 19.38 | | | | |
| 63 | AT- | protein | Light | Variable | DVQMTQTTSSLSASLGDRVTISC |
| | 19.38 | | | | RASQDINNYLHWYQQKPDGTVK |
| | | | | | LLIYYTSRLHSGVPSRFSGSGSGT |
| | | | | | DYSLTISNLDQEDIATYFCQQGK |
| | | | | | TLPWTFGGGTKLEIK |
| 64 | AT- | protein | Heavy | Variable | DVQLVESGGGLVKPGGSRKLSC |
| | 19.38 | | | | AASGFTFSDYGMHWVRQAPEKG |
| | | | | | LEWIAYISTGSGTIYYADTVKGR |
| | | | | | FTISRDNAKSTLFLQMTGLRSED |
| | | | | | TAMYYCARQGYSYTMDYWGQG |
| | | | | | TSVTVSS |
| 65 | AT- | protein | Light | CDR1 | KASQDVSTAVA |
| | 19.42 | | | | |
| 66 | AT- | protein | Light | CDR2 | SASYRST |
| | 19.42 | | | | |
| 67 | AT- | protein | Light | CDR3 | QQHFNTPRT |
| | 19.42 | | | | |
| 68 | AT- | protein | Heavy | CDR1 | SYAMS |
| | 19.42 | | | | |
| 69 | AT- | protein | Heavy | CDR2 | SINSGGFTYYPDSVKG |
| | 19.42 | | | | |
| 70 | AT- | protein | Heavy | CDR3 | GGTTVFDY |
| | 19.42 | | | | |
| 71 | AT- | protein | Light | Variable | DIVMTQSHKFMSTSVGDRVSITC |
| | 19.42 | | | | KASQDVSTAVAWYQQRPGHSPK |
| | | | | | LLIYSASYRSTGVPDRFTGSGSGT |
| | | | | | DFTFTISSVQAEDLAVYYCQQHF |
| | | | | | NTPRTFGGGTKLEIK |
| 72 | AT- | protein | Heavy | Variable | EVKLVESGGGLVKPGGSLKLSCA |
| | 19.42 | | | | ASGFTFSSYAMSWVRQTPEKRLE |
| | | | | | WVASINSGGFTYYPDSVKGRFTI |
| | | | | | SRDNARNILYLQMSSLRSEDTAM |
| | | | | | YYCARGGTTVFDYWGQGTTLTV |
| | | | | | SS |
| 73 | AT- | protein | Light | CDR1 | RASKSVSTSGYSYMH |
| | 19.80 | | | | |
| 74 | AT- | protein | Light | CDR2 | LVSNLES |
| | 19.80 | | | | |
| 75 | AT- | protein | Light | CDR3 | QQHFNTPRT |
| | 19.80 | | | | |
| 76 | AT- | protein | Heavy | CDR1 | SYAMS |
| | 19.80 | | | | |
| 77 | AT- | protein | Heavy | CDR2 | SINSGGFTYYPDSVKG |
| | 19.80 | | | | |
| 78 | AT- | protein | Heavy | CDR3 | GGTTVFDY |
| | 19.80 | | | | |
| 79 | AT- | protein | Light | Variable | DIVLTQSPASLAVSLGQRATISYR |
| | 19.80 | | | | ASKSVSTSGYSYMHWNQQKPGQ |
| | | | | | PPRLLIYLVSNLESGVPARFSGSG |
| | | | | | SGTDFTLNIHPVEEEDAATYYCQ |
| | | | | | QHFNTPRTFGGGTKLEIK |
| 80 | AT- | protein | Heavy | Variable | EVQLQESGGGLVKPGGSLKLSCA |
| | 19.80 | | | | ASGFTFSSYAMSWVRQTPEKRLE |
| | | | | | WVASINSGGFTYYPDSVKGRFTI |
| | | | | | SRDNARNILYLQMSSLRSEDTAM |
| | | | | | YYCARGGTTVFDYWGQGTTVT |
| | | | | | VSS |
| 81 | AT- | protein | Light | CDR1 | KSSQSLLNSGNQKNYLT |
| | 19.84 | | | | |
| 82 | AT- | protein | Light | CDR2 | WTSTRES |
| | 19.84 | | | | |
| 83 | AT- | protein | Light | CDR3 | QNDYSYPFT |
| | 19.84 | | | | |
| 84 | AT- | protein | Heavy | CDR1 | SNWIE |
| | 19.84 | | | | |
| 85 | AT- | protein | Heavy | CDR2 | EILPGSGRANNNEKFKG |
| | 19.84 | | | | |
| 86 | AT- | protein | Heavy | CDR3 | PNLFGGAMDY |
| | 19.84 | | | | |
| 87 | AT- | protein | Light | Variable | DIVMTQSPSSLTVTAGEKVTMSC |
| | 19.84 | | | | KSSQSLLNSGNQKNYLTWYQQK |
| | | | | | PGQPPKLLIYWTSTRESGVPARFT |
| | | | | | GSGSGTDFTLTISSVQAEDLAVY |
| | | | | | YCQNDYSYPFTFGSGTKLEIK |
| 88 | AT- | protein | Heavy | Variable | QVQLQQSGAELMKPGASVKISC |
| | 19.84 | | | | KATGYTISSNWIEWIKQRPGHGL |
| | | | | | EWIGEILPGSGRANNNEKFKGKA |
| | | | | | SFTADTSSNTAYIQLSSLTSEDSA |
| | | | | | VYYCARPNLFGGAMDYWGQGT |
| | | | | | SVTVSS |
| 89 | AT- | protein | Light | CDR1 | RASESVDTYGNSFMH |
| | 19.88 | | | | |
| 90 | AT- | protein | Light | CDR2 | RASNLES |
| | 19.88 | | | | |
| 91 | AT- | protein | Light | CDR3 | QQSNKDPWT |
| | 19.88 | | | | |
| 92 | AT- | protein | Heavy | CDR1 | SNWIE |
| | 19.88 | | | | |
| 93 | AT- | protein | Heavy | CDR2 | EILPGSGRANNNEKFKG |
| | 19.88 | | | | |
| 94 | AT- | protein | Heavy | CDR3 | PNLFGGAMDY |
| | 19.88 | | | | |
| 95 | AT- | protein | Light | Variable | DIVLTQSPASLAVSLGQRATISCR |
| | 19.88 | | | | ASESVDTYGNSFMHWYQQKPGQ |
| | | | | | PPKLLIYRASNLESGIPARFSGSG |
| | | | | | SRTDFTLTINPVEADDVATYYCQ |
| | | | | | QSNKDPWTFGGGTKLEIK |
| 96 | AT- | protein | Heavy | Variable | QVQLQQSGAELMKPGASVKISC |
| | 19.88 | | | | KATGYTISSNWIEWIKQRPGHGL |
| | | | | | EWIGEILPGSGRANNNEKFKGKA |
| | | | | | SFTADTSSNTAYIQLSSLTSEDSA |
| | | | | | VYYCARPNLFGGAMDYWGQGT |
| | | | | | SVTVSS |
| 97 | B1 | protein | Light | CDR1 | KASQDVYTAVA |
| 98 | B1 | protein | Light | CDR2 | SASYRYT |
| 99 | B1 | protein | Light | CDR3 | QQHYSAPRT |
| 100 | B1 | protein | Heavy | CDR1 | SYAMS |
| 101 | B1 | protein | Heavy | CDR2 | SINSGGITYSPDSVKG |
| 102 | B1 | protein | Heavy | CDR3 | GGTTFFDY |
| 103 | B1 | protein | Light | Variable | DIVMTQSHKFMSTSVGDRVSISC |
| | | | | | KASQDVYTAVAWYQQKPGQSP |
| | | | | | KLLIYSASYRYTGVPDRFTGSGS |
| | | | | | GTDFTFTISTVQAEDLAVYYCQQ |
| | | | | | HYSAPRTFGGGTKLEIK |
| 104 | B1 | protein | Heavy | Variable | EVKLMESGGGLVKPGGSLKLSC |
| | | | | | AASGFTFSSYAMSWVRQTPEKR |
| | | | | | LEWVASINSGGITYSPDSVKGRF |
| | | | | | TISRDNARNILYLQMSSLRSEDA |
| | | | | | AIYFCARGGTTFFDYWGQGTTLT |
| | | | | | VSS |
| 105 | B32 | protein | Light | CDR1 | KASQDVSTTVA |
| 106 | B32 | protein | Light | CDR2 | WASTRHT |
| 107 | B32 | protein | Light | CDR3 | QQHYSTPRT |
| 108 | B32 | protein | Heavy | CDR1 | SYAMS |
| 109 | B32 | protein | Heavy | CDR2 | SINSGGITYYPDSVKG |
| 110 | B32 | protein | Heavy | CDR3 | GGTTVFDY |
| 111 | B32 | protein | Light | Variable | DIVMTQSHKFMSTSVGDRVSITC |
| | | | | | KASQDVSTTVAWYQQKPGQSPK |
| | | | | | LLIYWASTRHTGVPDRFTGSGSG |
| | | | | | TDYTLTISPVQAEDLALYYCQQH |
| | | | | | YSTPRTFGGGTKLEIK |
| 112 | B32 | protein | Heavy | Variable | EMKLVESGGGLIKPGGSLKLSCA |
| | | | | | ASGFTFSSYAMSWVRQTPEKRLE |
| | | | | | WVASINSGGITYYPDSVKGRFTIS |
| | | | | | RDNARNILYLQMSSLRSEDTAM |
| | | | | | YYCARGGTTVFDYWGQGTTLTV |
| | | | | | SS |
| 113 | 16F7 | protein | Light | CDR1 | KASQDVSSAVA |
| 114 | 16F7 | protein | Light | CDR2 | WASTRHT |
| 115 | 16F7 | protein | Light | CDR3 | QQHFSSPRT |
| 116 | 16F7 | protein | Heavy | CDR1 | SYAMS |
| 117 | 16F7 | protein | Heavy | CDR2 | SLNSGGITYYPDSVRG |
| 118 | 16F7 | protein | Heavy | CDR3 | GGTTVFDY |
| 119 | 16F7 | protein | Light | Variable | DIVLTQSHKFMSTSVGDRVNITC |
| | | | | | KASQDVSSAVAWYQQKPGQSPK |
| | | | | | LLIYWASTRHTGVPDRFTGSGSG |
| | | | | | TDYTLTISTVQAEDLALYYCQQH |
| | | | | | FSSPRTFGGGTKLEIK |
| 120 | 16F7 | protein | Heavy | Variable | EVKLVESGGGLVRPGGSLKLSCA |
| | | | | | ASGFSFSSYAMSWVRQTPEKRL |
| | | | | | DWVASLNSGGITYYPDSVRGRFT |
| | | | | | ISRDIVRHTLYLQMSSLRSEDTA |
| | | | | | MYFCARGGTTVFDYWGQGTTLT |
| | | | | | VSS |
| 121 | 7F10 | protein | Light | CDR1 | SASSSISYIY |
| 122 | 7F10 | protein | Light | CDR2 | DTSNLAS |
| 123 | 7F10 | protein | Light | CDR3 | QQWSSYPPIT |
| 124 | 7F10 | protein | Heavy | CDR1 | AYTMN |
| 125 | 7F10 | protein | Heavy | CDR2 | LINPYNGDTVFNQKFKD |
| 126 | 7F10 | protein | Heavy | CDR3 | GLRTYWYFDV |
| 127 | 7F10 | protein | Light | Variable | QIVLTQSPAIMSASPGEKVTMTC |
| | | | | | SASSSISYIYWYQQKPGSSPRLLI |
| | | | | | YDTSNLASGVPVRFSGSGSGTSY |
| | | | | | SLTISRVEAEDAATYYCQQWSSY |
| | | | | | PPITFGGGTKLEIK |
| 128 | 7F10 | protein | Heavy | Variable | QLQQSGPEPVKPGTSMKISCKAS |
| | | | | | GYSFTAYTMNWVKQSHGKTLE |
| | | | | | WIGLINPYNGDTVFNQKFKDKAT |
| | | | | | LTVDRSSSTAFMELLSLTSDDSA |
| | | | | | VYYCARGLRTYWYFDVWGAGT |
| | | | | | TITVSS |
| 129 | AT- | DNA | Light | CDR1 | AAAAGCTCTCAATCCTTGCTGA |
| | 19.10 | | | | ACAGCGGAAACCAAAAGAAGT |
| | | | | | ACTTGACA |
| 130 | AT- | DNA | Light | CDR2 | TGGGCTGCAACACGGGAGAGC |
| | 19.10 | | | | |
| 131 | AT- | DNA | Light | CDR3 | CAGAACGACTACACCTATCCTC |
| | 19.10 | | | | TGACA |
| 132 | AT- | DNA | Heavy | CDR1 | AATTACTGGATTGAA |
| | 19.10 | | | | |
| 133 | AT- | DNA | Heavy | CDR2 | GAAATATTTCCAGGGAGCGGG |
| | 19.10 | | | | ACGACAAAGTATAACGAAAAG |
| | | | | | TTCAAAGGC |
| 134 | AT- | DNA | Heavy | CDR3 | GGCAGGTTTATCAGTACCACAA |
| | 19.10 | | | | GGGGATTCGCCTAT |
| 135 | AT- | DNA | Light | Variable | GACATCGTGATGACCCAATCAC |
| | 19.10 | | | | CAAGTAGCCTGACTGTGACGCC |
| | | | | | CGGGGAAAAGGTCACCATGTCC |
| | | | | | TGTAAAAGCTCTCAATCCTTGC |
| | | | | | TGAACAGCGGAAACCAAAAGA |
| | | | | | AGTACTTGACATGGTACCAACA |
| | | | | | GAAACCCGGTCAACCACCAAA |
| | | | | | GCTCTTGATATATTGGGCTGCA |
| | | | | | ACACGGGAGAGCGGTGTTCCA |
| | | | | | GACAGGTTCACCGGCAGTGGAT |
| | | | | | CTGGCACCGATTTTACCCTTAC |
| | | | | | GATCAGCTCCGTTCAGGCTGAA |
| | | | | | GACTTAGCTGTTTACTATTGTC |
| | | | | | AGAACGACTACACCTATCCTCT |
| | | | | | GACATTTGGGGCCGGGACAAA |
| | | | | | ATTAGAGCTTAAG |
| 136 | AT- | DNA | Heavy | Variable | CAAGTTCAACTGCAACAATCCG |
| | 19.10 | | | | GCGCAGAACTGACAAAACCTG |
| | | | | | GGGCATCCGTCAAAATTAGCTG |
| | | | | | TAAAGCGACTGGGTATACTTTC |
| | | | | | TCAAATTACTGGATTGAATGGG |
| | | | | | TTATTCAGCGGCCAGGTCATGG |
| | | | | | CCTTGAATGGATTGGCGAAATA |
| | | | | | TTTCCAGGGAGCGGGACGACA |
| | | | | | AAGTATAACGAAAAGTTCAAA |
| | | | | | GGCAAAGCCACATTCACAGCCG |
| | | | | | ACACGTCTAGTAATACTGCGTA |
| | | | | | TATGCAATTAAATAGTTTAACC |
| | | | | | TCAGAAGATTCCGCGGTGTATT |
| | | | | | ATTGCGCACGGGGCAGGTTTAT |
| | | | | | CAGTACCACAAGGGGATTCGCC |
| | | | | | TATTGGGGCCAAGGGACATTGG |
| | | | | | TAACAGTAAGTGCC |
| 137 | AT- | DNA | Light | CDR1 | AAAGCTTCTCAGGATGTAAGTA |
| | 19.20 | | | | CAGCCGTCGCG |
| 138 | AT- | DNA | Light | CDR2 | TCCGCGAGCTATCGATATACT |
| | 19.20 | | | | |
| 139 | AT- | DNA | Light | CDR3 | CAACAACACTTTAATACACCAC |
| | 19.20 | | | | GAACC |
| 140 | AT- | DNA | Heavy | CDR1 | AGTTATGCGATGTCA |
| | 19.20 | | | | |
| 141 | AT- | DNA | Heavy | CDR2 | TCTATTAATAGTGGCGGTTTTA |
| | 19.20 | | | | CCTACTATCCTGACAGTGTCAA |
| | | | | | AGGC |
| 142 | AT- | DNA | Heavy | CDR3 | GGCGGGACAACCGTCTTCGACT |
| | 19.20 | | | | AC |
| 143 | AT- | DNA | Light | Variable | GATATTGTTATGACTCAATCCC |
| | 19.20 | | | | ACAAATTTATGTCTACAAGTGT |
| | | | | | AGGCGACAGGGTCAGCATCAC |
| | | | | | CTGCAAAGCTTCTCAGGATGTA |
| | | | | | AGTACAGCCGTCGCGTGGTATC |
| | | | | | AACAGAAACCTGGACACAGCC |
| | | | | | CAAAGTTGCTGATATACTCCGC |
| | | | | | GAGCTATCGATATACTGGTGTG |
| | | | | | CCCGACAGGTTTACAGGGTCAG |
| | | | | | GTAGCGGCGCGGATTTCACGTT |
| | | | | | TACAATCAGCTCTGTACAGGCA |
| | | | | | GAAGACTTAGCCGTGTACTTTT |
| | | | | | GTCAACAACACTTTAATACACC |
| | | | | | ACGAACCTTTGGCGGCGGTACC |
| | | | | | AAACTGGAGATAAAG |
| 144 | AT- | DNA | Heavy | Variable | GAAGTGAAACTTGTGGAGTCTG |
| | 19.20 | | | | GTGGTGGATTGGTTAAGCCCGG |
| | | | | | TGGTAGCTTGAAACTGTCCTGC |
| | | | | | GCAGCGTCTGGCTTTACTTTCTC |
| | | | | | CAGTTATGCGATGTCATGGGTG |
| | | | | | CGCCAAACACCTGAGAAGAGG |
| | | | | | CTGGAGTGGGTAGCCTCTATTA |
| | | | | | ATAGTGGCGGTTTTACCTACTA |
| | | | | | TCCTGACAGTGTCAAAGGCAGG |
| | | | | | TTTACAATTTCTCGCGACAACA |
| | | | | | CCAGAAATATCTTGTATCTTCA |
| | | | | | ACTCAGTAGCTTGAGGTCTGAA |
| | | | | | GACACGGCAATGTATTACTGTG |
| | | | | | CCCGAGGCGGGACAACCGTCTT |
| | | | | | CGACTACTGGGGACAAGGGAC |
| | | | | | GACCTTGACGGTCTCATCA |
| 145 | AT- | DNA | Light | CDR1 | AAAGCCAGTCAGAACGTAGGA |
| | 19.21 | | | | ACCAATGTCGCC |
| 146 | AT- | DNA | Light | CDR2 | TCAGCCAGCTATCGATATTCC |
| | 19.21 | | | | |
| 147 | AT- | DNA | Light | CDR3 | CAACAATATAACAGCTACCCCC |
| | 19.21 | | | | TCACA |
| 148 | AT- | DNA | Heavy | CDR1 | TCTTATGCAATGAGT |
| | 19.21 | | | | |
| 149 | AT- | DNA | Heavy | CDR2 | ACCATATCAAGCGGTGGAAGTT |
| | 19.21 | | | | ACACATATTACTTAGACAGTGT |
| | | | | | GAAAGGA |
| 150 | AT- | DNA | Heavy | CDR3 | CCACCTGCACGGGCCGGTATCT |
| | 19.21 | | | | ATTACTACGTGATGGACTAT |
| 151 | AT- | DNA | Light | Variable | GACATCGTCATGACACAGTCAC |
| | 19.21 | | | | AGAAATTTATGTCTACATCAGT |
| | | | | | GGGCGATCGGGTTAGCGTCACC |
| | | | | | TGTAAAGCCAGTCAGAACGTAG |
| | | | | | GAACCAATGTCGCCTGGTATCA |
| | | | | | ACAGAAACCTGGCCAAAGTCCC |
| | | | | | AAACCTCTTATTTACTCAGCCA |
| | | | | | GCTATCGATATTCCGGAGTCCC |
| | | | | | AGATCGGTTTACTGGAAGTGGA |
| | | | | | AGCGGAACTGACTTTACCCTCA |
| | | | | | CTATTAGTAACGTTCAATCAGA |
| | | | | | AGATTTAGCCGAGTATTTCTGC |
| | | | | | CAACAATATAACAGCTACCCCC |
| | | | | | TCACATTTGGGGCAGGCACTAA |
| | | | | | GTTAGAGTTGAAG |
| 152 | AT- | DNA | Heavy | Variable | GAAGTACAACTTGTTGAGTCTG |
| | 19.21 | | | | GCGGCGGTCTGGTTAAACCTGG |
| | | | | | CGGATCCCTTAAATTGAGTTGT |
| | | | | | GCCGCTTCCGGGTTCACGTTCA |
| | | | | | GCTCTTATGCAATGAGTTGGGT |
| | | | | | CCGTCAGACCCCGGAAAAGCG |
| | | | | | CCTCGAATGGGTCGCCACCATA |
| | | | | | TCAAGCGGTGGAAGTTACACAT |
| | | | | | ATTACTTAGACAGTGTGAAAGG |
| | | | | | ACGATTCACCATTTCAAGAGAC |
| | | | | | AACGCTAAGAACACTTTGTATT |
| | | | | | TACAAATGTCTAGCTTAAGGTC |
| | | | | | AGAAGATACTGCCATGTATTAT |
| | | | | | TGTGCAAGACCACCTGCACGGG |
| | | | | | CCGGTATCTATTACTACGTGAT |
| | | | | | GGACTATTGGGGCCAAGGAAC |
| | | | | | GAGCGTCACAGTGTCATCA |
| 153 | AT- | DNA | Light | CDR1 | CGC GCA AGT CAG GAC ATC |
| | 19.38 | | | | AAT AAT TAT CTG CAC |
| 154 | AT- | DNA | Light | CDR2 | TAC ACA TCA AGG CTG CAT AGC |
| | 19.38 | | | | |
| 155 | AT- | DNA | Light | CDR3 | CAA CAA GGA AAG ACA CTG |
| | 19.38 | | | | CCG TGG ACC |
| 156 | AT- | DNA | Heavy | CDR1 | GAT TAC GGG ATG CAT |
| | 19.38 | | | | |
| 157 | AT- | DNA | Heavy | CDR2 | TAT ATT TCC ACT GGA AGT GGT |
| | 19.38 | | | | ACG ATT TAT TAT |
| | | | | | GCG GAT ACA GTC AAG GGT |
| 158 | AT- | DNA | Heavy | CDR3 | CAG GGA |
| | 19.38 | | | | TAC AGC TAT ACA ATG GAC TAT |
| 159 | AT- | DNA | Light | Variable | GACGTTCAAATGACCCAAACCA |
| | 19.38 | | | | CTTCCAGCTTGTCCGCCTCTCTG |
| | | | | | GGTGATAGAGTAACGATCTCCT |
| | | | | | GCCGCGCAAGTCAGGACATCA |
| | | | | | ATAATTATCTGCACTGGTATCA |
| | | | | | ACAAAAGCCGGATGGAACAGT |
| | | | | | AAAGCTGCTTATATACTACACA |
| | | | | | TCAAGGCTGCATAGCGGCGTCC |
| | | | | | CCAGCAGGTTCTCAGGATCAGG |
| | | | | | GTCCGGGACAGACTACTCTCTT |
| | | | | | ACCATATCAAACCTCGACCAAG |
| | | | | | AAGATATCGCAACATACTTCTG |
| | | | | | CCAACAAGGAAAGACACTGCC |
| | | | | | GTGGACCTTCGGAGGAGGAAC |
| | | | | | AAAACTCGAAATCAAA |
| 160 | AT- | DNA | Heavy | Variable | GATGTACAATTGGTGGAAAGTG |
| | 19.38 | | | | GAGGAGGCTTGGTCAAACCGG |
| | | | | | GTGGAAGTAGGAAACTCAGCT |
| | | | | | GCGCCGCTTCAGGATTTACATT |
| | | | | | CTCCGATTACGGGATGCATTGG |
| | | | | | GTTCGCCAGGCCCCAGAAAAG |
| | | | | | GGCCTTGAGTGGATCGCGTATA |
| | | | | | TTTCCACTGGAAGTGGTACGAT |
| | | | | | TTATTATGCGGATACAGTCAAG |
| | | | | | GGTAGGTTCACCATTTCCCGCG |
| | | | | | ACAACGCAAAGAGCACCCTCTT |
| | | | | | TCTTCAAATGACCGGCTTGAGA |
| | | | | | AGCGAGGACACCGCCATGTATT |
| | | | | | ATTGCGCCAGGCAGGGATACA |
| | | | | | GCTATACAATGGACTATTGGGG |
| | | | | | ACAAGGAACGAGTGTTACAGTT |
| | | | | | AGTAGT |
| 161 | AT- | DNA | Light | CDR1 | AAA GCA TCT CAA GAC GTC TCC |
| | 19.42 | | | | ACA GCT GTA GCC |
| 162 | AT- | DNA | Light | CDR2 | AGT GCT AGT TAC AGG TCC ACA |
| | 19.42 | | | | |
| 163 | AT- | DNA | Light | CDR3 | CAG CAG CAC TTT AAC ACC CCT |
| | 19.42 | | | | CGG ACG |
| 164 | AT- | DNA | Heavy | CDR1 | AGC TAT GCG ATG TCA |
| | 19.42 | | | | |
| 165 | AT- | DNA | Heavy | CDR2 | TCT ATA AAC TCA GGT GGG TTT |
| | 19.42 | | | | ACC TAT TAC CCA GAT TCA GTT |
| | | | | | AAA GGT |
| 166 | AT- | DNA | Heavy | CDR3 | GGT GGG ACC |
| | 19.42 | | | | ACC GTT TTC GAC TAC |
| 167 | AT- | DNA | Light | Variable | GATATCGTCATGACCCAAAGCC |
| | 19.42 | | | | ACAAATTTATGTCCACCAGTGT |
| | | | | | CGGGGACAGAGTGAGTATTACT |
| | | | | | TGTAAAGCATCTCAAGACGTCT |
| | | | | | CCACAGCTGTAGCCTGGTATCA |
| | | | | | ACAACGGCCCGGTCATAGCCCC |
| | | | | | AAACTGCTCATTTACAGTGCTA |
| | | | | | GTTACAGGTCCACAGGAGTACC |
| | | | | | TGATCGCTTTACGGGCAGTGGG |
| | | | | | TCAGGCACTGATTTCACTTTTA |
| | | | | | CTATCTCATCCGTGCAGGCTGA |
| | | | | | GGACTTGGCGGTATATTACTGC |
| | | | | | CAGCAGCACTTTAACACCCCTC |
| | | | | | GGACGTTCGGCGGTGGGACCA |
| | | | | | AACTGGAGATCAAA |
| 168 | AT- | DNA | Heavy | Variable | GAAGTCAAACTCGTAGAAAGC |
| | 19.42 | | | | GGCGGCGGCCTCGTAAAACCCG |
| | | | | | GCGGGTCACTGAAGCTCAGTTG |
| | | | | | TGCGGCGTCAGGATTCACATTC |
| | | | | | TCAAGCTATGCGATGTCATGGG |
| | | | | | TCAGGCAAACTCCAGAGAAAA |
| | | | | | GATTGGAATGGGTGGCATCTAT |
| | | | | | AAACTCAGGTGGGTTTACCTAT |
| | | | | | TACCCAGATTCAGTTAAAGGTC |
| | | | | | GGTTTACAATTTCCAGGGACAA |
| | | | | | TGCGCGGAATATCCTGTACCTG |
| | | | | | CAAATGAGCAGCCTGCGCAGC |
| | | | | | GAAGACACGGCAATGTACTACT |
| | | | | | GTGCTCGCGGTGGGACCACCGT |
| | | | | | TTTCGACTACTGGGGACAAGGG |
| | | | | | ACTACACTGACAGTTTCTAGT |
| 169 | AT- | DNA | Light | CDR1 | CGAGCCTCTAAAAGCGTGAGCA |
| | 19.80 | | | | CGAGCGGCTATAGCTACATGCA |
| | | | | | C |
| 170 | AT- | DNA | Light | CDR2 | CTGGTCTCCAATTTGGAGTCC |
| | 19.80 | | | | |
| 171 | AT- | DNA | Light | CDR3 | CAACAACACTTCAATACCCCGC |
| | 19.80 | | | | GCACC |
| 172 | AT- | DNA | Heavy | CDR1 | TCATATGCAATGTCA |
| | 19.80 | | | | |
| 173 | AT- | DNA | Heavy | CDR2 | AGTATCAATTCCGGCGGTTTCA |
| | 19.80 | | | | CTTATTACCCTGATTCTGTGAA |
| | | | | | GGGT |
| 174 | AT- | DNA | Heavy | CDR3 | GGTGGAACTACCGTCTTCGATT |
| | 19.80 | | | | AC |
| 175 | AT- | DNA | Light | Variable | GATATCGTGCTGACTCAATCTC |
| | 19.80 | | | | CTGCCTCTTTAGCGGTTAGCCT |
| | | | | | GGGCCAAAGGGCAACTATCTCT |
| | | | | | TACCGAGCCTCTAAAAGCGTGA |
| | | | | | GCACGAGCGGCTATAGCTACAT |
| | | | | | GCACTGGAATCAACAGAAACCT |
| | | | | | GGCCAACCGCCCAGACTCTTGA |
| | | | | | TTTATCTGGTCTCCAATTTGGA |
| | | | | | GTCCGGTGTCCCGGCCCGATTC |
| | | | | | TCAGGATCCGGTAGCGGGACTG |
| | | | | | ATTTTACGCTCAACATACATCC |
| | | | | | TGTTGAAGAGGAAGACGCTGCC |
| | | | | | ACTTATTACTGTCAACAACACT |
| | | | | | TCAATACCCCGCGCACCTTCGG |
| | | | | | CGGCGGCACTAAACTCGAGATC |
| | | | | | AAG |
| 176 | AT- | DNA | Heavy | Variable | GAGGTCCAATTACAAGAGAGT |
| | 19.80 | | | | GGTGGCGGATTGGTTAAACCAG |
| | | | | | GTGGATCTTTAAAGCTTAGCTG |
| | | | | | TGCCGCAAGCGGCTTTACCTTT |
| | | | | | TCATCATATGCAATGTCATGGG |
| | | | | | TGCGACAAACCCCCGAGAAAC |
| | | | | | GTTTGGAATGGGTTGCCAGTAT |
| | | | | | CAATTCCGGCGGTTTCACTTAT |
| | | | | | TACCCTGATTCTGTGAAGGGTC |
| | | | | | GCTTTACCATCTCTAGAGATAA |
| | | | | | CGCTAGAAACATCTTGTACCTG |
| | | | | | CAAATGAGTAGCCTGCGTAGTG |
| | | | | | AAGACACTGCAATGTACTATTG |
| | | | | | CGCCCGCGGTGGAACTACCGTC |
| | | | | | TTCGATTACTGGGGTCAAGGTA |
| | | | | | CCACTGTGACAGTATCATCA |
| 177 | AT- | DNA | Light | CDR1 | AAATCTAGCCAATCTCTTTTAA |
| | 19.84 | | | | ATTCTGGAAACCAGAAGAACTA |
| | | | | | TCTGACA |
| 178 | AT- | DNA | Light | CDR2 | TGGACAAGCACAAGG |
| | 19.84 | | | | |
| 179 | AT- | DNA | Light | CDR3 | CAAAACGATTACTCTTACCCTT |
| | 19.84 | | | | TCACG |
| 180 | AT- | DNA | Heavy | CDR1 | TCAAACTGGATTGAG |
| | 19.84 | | | | |
| 181 | AT- | DNA | Heavy | CDR2 | GAGATTCTTCCAGGAAGTGGGC |
| | 19.84 | | | | GGGCAAACAACAATGAGAAAT |
| | | | | | TTAAGGGC |
| 182 | AT- | DNA | Heavy | CDR3 | CCCAACCTCTTTGGTGGAGCTA |
| | 19.84 | | | | TGGATTAT |
| 183 | AT- | DNA | Light | Variable | GATATCGTGATGACACAAAGTC |
| | 19.84 | | | | CTTCATCTCTGACAGTTACAGC |
| | | | | | TGGGGAGAAAGTCACCATGTCC |
| | | | | | TGTAAATCTAGCCAATCTCTTTT |
| | | | | | AAATTCTGGAAACCAGAAGAA |
| | | | | | CTATCTGACATGGTACCAACAG |
| | | | | | AAACCGGGCCAACCCCCAAAG |
| | | | | | CTGCTCATTTATTGGACAAGCA |
| | | | | | CAAGGGAGAGCGGGGTTCCAG |
| | | | | | CGAGATTTACAGGGTCTGGCTC |
| | | | | | AGGAACGGATTTCACACTGACC |
| | | | | | ATCTCATCCGTGCAAGCTGAAG |
| | | | | | ACCTGGCAGTTTACTACTGTCA |
| | | | | | AAACGATTACTCTTACCCTTTC |
| | | | | | ACGTTCGGCAGCGGAACCAAGT |
| | | | | | TGGAAATCAAA |
| 184 | AT- | DNA | Heavy | Variable | CAAGTACAGCTGCAACAATCAG |
| | 19.84 | | | | GCGCGGAGCTGATGAAACCCG |
| | | | | | GTGCCAGCGTCAAGATTTCTTG |
| | | | | | CAAAGCCACCGGCTATACAATT |
| | | | | | AGCTCAAACTGGATTGAGTGGA |
| | | | | | TTAAACAACGGCCCGGGCACG |
| | | | | | GACTCGAATGGATAGGAGAGA |
| | | | | | TTCTTCCAGGAAGTGGGCGGGC |
| | | | | | AAACAACAATGAGAAATTTAA |
| | | | | | GGGCAAGGCATCCTTCACCGCA |
| | | | | | GATACGTCTTCCAACACGGCTT |
| | | | | | ACATTCAATTGAGCTCTCTCAC |
| | | | | | ATCTGAAGACTCCGCCGTATAT |
| | | | | | TATTGCGCCCGGCCCAACCTCT |
| | | | | | TTGGTGGAGCTATGGATTATTG |
| | | | | | GGGCCAGGGGACCAGCGTGAC |
| | | | | | CGTTAGCAGC |
| 185 | AT- | DNA | Light | CDR1 | CGGGCCTCTGAATCAGTAGACA |
| | 19.88 | | | | CTTACGGGAACAGTTTTATGCA |
| | | | | | C |
| 186 | AT- | DNA | Light | CDR2 | AGAGCTTCCAACCTCGAATCC |
| | 19.88 | | | | |
| 187 | AT- | DNA | Light | CDR3 | CAGCAAAGCAACAAAGACCCG |
| | 19.88 | | | | TGGACA |
| 188 | AT- | DNA | Heavy | CDR1 | TCAAACTGGATTGAG |
| | 19.88 | | | | |
| 189 | AT- | DNA | Heavy | CDR2 | GAGATTCTTCCAGGAAGTGGGC |
| | 19.88 | | | | GGGCAAACAACAATGAGAAAT |
| | | | | | TTAAGGGC |
| 190 | AT- | DNA | Heavy | CDR3 | CCCAACCTCTTTGGTGGAGCTA |
| | 19.88 | | | | TGGATTAT |
| 191 | AT- | DNA | Light | Variable | GATATCGTTTTGACACAAAGTC |
| | 19.88 | | | | CAGCCAGTCTCGCCGTTTCCTT |
| | | | | | GGGCCAACGGGCTACTATTAGT |
| | | | | | TGCCGGGCCTCTGAATCAGTAG |
| | | | | | ACACTTACGGGAACAGTTTTAT |
| | | | | | GCACTGGTATCAACAGAAACCC |
| | | | | | GGCCAGCCCCCAAAACTCTTGA |
| | | | | | TATACAGAGCTTCCAACCTCGA |
| | | | | | ATCCGGGATCCCGGCTCGGTTT |
| | | | | | AGTGGGTCCGGATCCAGAACA |
| | | | | | GATTTCACTCTTACCATCAATC |
| | | | | | CTGTTGAGGCCGATGATGTTGC |
| | | | | | CACCTACTACTGCCAGCAAAGC |
| | | | | | AACAAAGACCCGTGGACATTCG |
| | | | | | GCGGTGGGACCAAACTGGAAA |
| | | | | | TAAAG |
| 192 | AT- | DNA | Heavy | Variable | CAAGTACAGCTGCAACAATCAG |
| | 19.88 | | | | GCGCGGAGCTGATGAAACCCG |
| | | | | | GTGCCAGCGTCAAGATTTCTTG |
| | | | | | CAAAGCCACCGGCTATACAATT |
| | | | | | AGCTCAAACTGGATTGAGTGGA |
| | | | | | TTAAACAACGGCCCGGGCACG |
| | | | | | GACTCGAATGGATAGGAGAGA |
| | | | | | TTCTTCCAGGAAGTGGGCGGGC |
| | | | | | AAACAACAATGAGAAATTTAA |
| | | | | | GGGCAAGGCATCCTTCACCGCA |
| | | | | | GATACGTCTTCCAACACGGCTT |
| | | | | | ACATTCAATTGAGCTCTCTCAC |
| | | | | | ATCTGAAGACTCCGCCGTATAT |
| | | | | | TATTGCGCCCGGCCCAACCTCT |
| | | | | | TTGGTGGAGCTATGGATTATTG |
| | | | | | GGGCCAGGGGACCAGCGTGAC |
| | | | | | CGTTAGCAGC |
| 193 | ALT- | protein | Light | Variable | MGWSCIILFLVATATGVHSDVV |
| | anti- | | | | MTQSPLSLPVTPGEPASISCRSSQ |
| | FLT3 | | | | SLLHSNGNNYLDWYLQKPGQSP |
| | | | | | QLLIYLGSNRASGVPDRFSGSGS |
| | | | | | DTDFTLQISRVEAEDVGVYYCM |
| | | | | | QGTHPAISFGQGTRLEIKRTVAAP |
| | | | | | SVFIFPPSDEQLKSGTASVVCLLN |
| | | | | | NFYPREAKVQWKVDNALQSGNS |
| | | | | | QESVTEQDSKDSTYSLSSTLTLSK |
| | | | | | ADYEKHKVYACEVTHQGLSSPV |
| | | | | | TKSFNRGEC |
| 194 | ALT- | protein | Heavy | Variable | MGWSCIILFLVATATGVHSEVQL |
| | anti- | | | | VQSGAEVKKPGASVKVSCKASG |
| | FLT3 | | | | YTFTSYYMHWVRQAPGQGLEW |
| | | | | | MGIINPSGGSTSYAQKFQGRVTM |
| | | | | | TRDTSTSTVYMELSSLRSEDTAV |
| | | | | | YYCARGVGAHDAFDIWGQGTTV |
| | | | | | TVSSASTKGPSVFPLAPSSKSTSG |
| | | | | | GTAALGCLVKDYFPEPVTVSWN |
| | | | | | SGALTSGVHTFPAVLQSSGLYSL |
| | | | | | SSVVTVPSSSLGTQTYICNVNHK |
| | | | | | PSNTKVDKRVEPKSCDKTHTCPP |
| | | | | | CPAPELLGGPSVFLFPPKPKDTL |
| | | | | | MISRTPEVTCVVVDVSHEDPEVK |
| | | | | | FNWYVDGVEVHNAKTKPREEQ |
| | | | | | YNSTYRVVSVLTVLHQDWLNGK |
| | | | | | EYKCKVSNKALPAPIEKTISKAK |
| | | | | | GQPREPQVYTLPPSREEMTKNQV |
| | | | | | SLTCLVKGFYPSDIAVEWESNGQ |
| | | | | | PENNYKTTPPVLDSDGSFFLYSK |
| | | | | | LTVDKSRWQQGNVFSCSVMHEA |
| | | | | | LHNHYTQKSLSLSPGK |
| 195 | ALT- | DNA | Light | Variable | atgggatggtcatgtatcatcctttttctagtagcaac |
| | anti- | | | | tgcaactggagtacattcagatgttgtgatgactcag |
| | FLT3 | | | | tctccactctccctgcccgtcacccctggagagccg |
| | | | | | gcctccatctcctgcaggtctagtcagagcctcctg |
| | | | | | catagtaatggaaacaactatttggattggtacctgc |
| | | | | | agaagccagggcagtctccacagctcctgatctatt |
| | | | | | tgggttctaatcgggcctctggggtcccagacagat |
| | | | | | tcagcggcagtgggtcagacactgatttcacactgc |
| | | | | | aaatcagtagggtggaggctgaggatgttggggttt |
| | | | | | attactgcatgcaaggtacacaccccgccatctcctt |
| | | | | | cggccaagggacacgactggagattaaacgtacg |
| | | | | | gtggctgcaccatctgtcttcatcttcccgccatctg |
| | | | | | atgagcagttgaaatctggaactgcctctgttgtgtg |
| | | | | | cctgctgaataacttctatcccagagaggccaaagt |
| | | | | | acagtggaaggtggataacgccctccaatcgggta |
| | | | | | actcccaggagagtgtcacagagcaggacagcaa |
| | | | | | ggacagcacctacagcctcagcagcaccctgacg |
| | | | | | ctgagcaaagcagactacgagaaacacaaagtct |
| | | | | | acgcctgcgaagtcacccatcagggcctgagctc |
| | | | | | gcccgtcacaaagagcttcaacaggggagagtgtt |
| | | | | | ag |
| 196 | ALT- | DNA | Heavy | Variable | atgggatggtcatgtatcatcctttttctagtagcaac |
| | anti- | | | | tgcaactggagtacattcagaggtccagctggtgc |
| | FLT3 | | | | agtctggggctgaggtgaagaagcctggggcctc |
| | | | | | agtgaaggtttcctgcaaggcatctggatacacctt |
| | | | | | caccagctactatatgcactgggtgcgacaggccc |
| | | | | | ctggacaagggcttgagtggatgggaataatcaac |
| | | | | | cctagtggtggtagcacaagctacgcacagaagtt |
| | | | | | ccagggcagagtcaccatgaccagggacacgtcc |
| | | | | | acgagcacagtctacatggagctgagcagcctga |
| | | | | | gatctgaggacacggccgtgtattactgtgcgagg |
| | | | | | ggagtgggagcgcatgatgcttttgatatctggggc |
| | | | | | caagggaccacggtcaccgtctcaagcgctagca |
| | | | | | ccaagggcccatcggtcttccccctggcaccctcct |
| | | | | | ccaagagcacctctgggggcacagcggccctgg |
| | | | | | gctgcctggtcaaggactacttccccgaaccggtg |
| | | | | | acggtgtcgtggaactcaggcgccctgaccagcg |
| | | | | | gcgtgcacaccttcccggctgtcctacagtcctcag |
| | | | | | gactctactccctcagcagcgtggtgaccgtgccct |
| | | | | | ccagcagcttgggcacccagacctacatctgcaac |
| | | | | | gtgaatcacaagcccagcaacaccaaggtggaca |
| | | | | | agagagttgagcccaaatcttgtgacaaaactcaca |
| | | | | | catgcccaccgtgcccagcacctgaactcctgggg |
| | | | | | ggaccgtcagtcttcctcttccccccaaaacccaag |
| | | | | | gacaccctcatgatctcccggacccctgaggtcac |
| | | | | | atgcgtggtggtggacgtgagccacgaagaccct |
| | | | | | gaggtcaagttcaactggtatgtggacggcgtgga |
| | | | | | ggtgcataatgccaagacaaagccgcgggagga |
| | | | | | gcagtacaacagcacgtaccgtgtggtcagcgtcc |
| | | | | | tcaccgtcctgcaccaagactggctgaatggcaag |
| | | | | | gagtacaagtgcaaggtctccaacaaagccctccc |
| | | | | | agcccccatcgagaaaaccatctccaaagccaaa |
| | | | | | gggcagccccgagaaccacaggtgtacaccctgc |
| | | | | | ccccatcccgggaggagatgaccaagaaccaagt |
| | | | | | cagcctgacctgcctggtcaaaggcttctatcccag |
| | | | | | cgacatcgccgtggagtgggagagcaatgggca |
| | | | | | gccggagaacaactacaagaccacgcctcccgtg |
| | | | | | ctggactccgacggctccttcttcctctattccaagct |
| | | | | | caccgtggacaagagcaggtggcagcaggggaa |
| | | | | | cgtcttctcatgctccgtgatgcatgaggctctgcac |
| | | | | | aaccactacacgcagaagagcctctccctgtctcc |
| | | | | | gggcaaatga |

### NUMBERED EMBODIMENTS

Embodiment 1. An anti-fms-like tyrosine kinase 3 (FLT3) antibody comprising a light chain variable domain and a heavy chain variable domain, wherein the light chain variable domain comprises: a light chain CDR1 having at least 90% sequence identity to a light chain CDR1 (CDR L1) amino acid sequence selected from Table 2, a light chain CDR2 (CDR L2) having at least 90% sequence identity to a light chain CDR2 amino acid sequence selected from Table 2, and a light chain CDR3 (CDR L3) having at least 90% sequence identity to a light chain CDR3 amino acid sequence selected from Table 2; and wherein the heavy chain variable domain comprises: a heavy chain CDR1 having at least 90% sequence identity to a heavy chain CDR1 (CDR H1) amino acid sequence selected from Table 2, a heavy chain CDR2 (CDR H2) having at least 90% sequence identity to a heavy chain CDR2 amino acid sequence selected from Table 2, and a CDR3 (CDR H3) having at least 90% sequence identity to a heavy chain CDR3 amino acid sequence selected from Table 2.
Embodiment 2. An anti-fms-like tyrosine kinase 3 (FLT3) antibody comprising a light chain variable domain and a heavy chain variable domain, wherein the light chain variable domain comprises: a light chain CDR1 (CDR L1) as set forth in SEQ ID NO:9, a light chain CDR2 (CDR L2) as set forth in SEQ ID NO:10 and a light chain CDR3 (CDR L3) as set forth in SEQ ID NO:11; and wherein the heavy chain variable domain comprises: a heavy chain CDR1 (CDR H1) as set forth in SEQ ID NO:12, a heavy chain (CDR H2) as set forth in SEQ ID NO:13, and a heavy chain (CDR H3) as set forth in SEQ ID NO:14.
Embodiment 3. The antibody of embodiment 1 or embodiment 2, wherein the light chain variable domain comprises an amino acid sequence with at least 80%, 85%, 90%, 95%, 98%, 99%, or 100% sequence identity with a light chain variable domain sequence selected from Table 2.
Embodiment 4. The antibody of embodiment 2 or embodiment 3, wherein the heavy chain variable domain comprises an amino acid sequence with at least 80%, 85%, 90%, 95%, 98%, 99%, or 100% sequence identity with a heavy chain variable domain sequence selected from Table 2.
Embodiment 5. The antibody of embodiment 1 or embodiment 2, wherein the antibody is a humanized antibody.
Embodiment 6. The antibody of any one of embodiments 1-4, wherein the antibody is a chimeric antibody.
Embodiment 7. The antibody of any one of embodiments 1-6, wherein the antibody is an IgG.
Embodiment 8. The antibody of embodiment 7, wherein the antibody is an IgG1.
Embodiment 9. The antibody of any one of embodiments 1-5, wherein the antibody is a Fab′ fragment.
Embodiment 10. The antibody of any one of embodiments 1-4, wherein the antibody is a single chain antibody (scFv).
Embodiment 11. The antibody of any one of embodiments 1-4, wherein the light chain variable domain and the heavy chain variable domain form part of a scFv.
Embodiment 12. The antibody of any one of embodiments 1-11, wherein the antibody is capable of binding FLT3.
Embodiment 13. The antibody of any one of embodiments 1-12, wherein the antibody is bound to FLT3.
Embodiment 14. The antibody of embodiment 12 or embodiment 13, wherein the FLT3 forms part of a cell.
Embodiment 15. The antibody of embodiment 14, wherein the cell is selected from a cancer cell, a myeloid cell, or a stem cell.
Embodiment 16. The antibody of embodiment 15, wherein the cancer cell is an acute myeloid leukemia cell.
Embodiment 17. An anti-FLT3 antibody, wherein the anti-FLT3 antibody binds the same epitope as an antibody comprising: a heavy chain variable region domain comprising a CDR1 (CDR H1) as set forth in Table 2, a CDR2 (CDR H2) as set forth in Table 2, and a CDR3 (CDR H3) as set forth in Table 2, and a light chain variable domain comprising a CDR1 (CDR L1) as set forth in Table 2, a CDR2 (CDR L2) as set forth in Table 2, and a CDR3 (CDR L3) as set forth in Table 2.
Embodiment 18. The anti-FLT3 antibody of embodiment 17, wherein the light chain variable domain comprises an amino acid sequence with at least 80%, 85%, 90%, 95%, 98%, 99%, or 100% sequence identity with a light chain variable domain amino acid sequence as set forth in Table 2.
Embodiment 19. The anti-FLT3 antibody of embodiment 17 or embodiment 18, wherein the heavy chain variable domain comprises an amino acid sequence with at least 80%, 85%, 90%, 95%, 98%, 99%, or 100% sequence identity with a light chain variable domain amino acid sequence as set forth in Table 2.
Embodiment 20. The anti-FLT3 antibody of any one of the preceding embodiments, wherein the antibody is conjugated to a payload.
Embodiment 21. The anti-FLT3 antibody of embodiment 20, wherein the payload is an antineoplastic agent.
Embodiment 22. The anti-FLT3 antibody of embodiment 20 or embodiment 21, wherein the payload is selected from monomethyl auristatin E (MMAE), DXd, or DX8951.
Embodiment 23. The anti-FLT3 antibody of any one of the preceding embodiments, wherein the antibody is conjugated to a linker molecule.
Embodiment 24. The anti-FLT3 antibody of embodiment 23, wherein the linker is selected from MC-Val-Cit-PAB or a tetrapeptide.
Embodiment 25. The anti-FLT3 antibody of embodiment 24, wherein the tetrapeptide is GGFG.
Embodiment 26. An anti-FLT3 antibody, the antibody comprising: a heavy chain variable region according to SEQ ID NO: 194; a light chain variable region according to SEQ ID NO: 193; and a drug payload.
Embodiment 27. The anti-FLT3 antibody of embodiment 26, wherein the drug payload is selected from DXd or DX8951.
Embodiment 28. The anti-FLT3 antibody of embodiment 26 or embodiment 27, wherein the drug payload is directly conjugated to the anti-FLT3 antibody via a linker.
Embodiment 29. The anti-FLT3 antibody of embodiment 28, wherein the linker is GGFG.
Embodiment 30. A kit comprising: the anti-FLT3 antibody of any one of the preceding embodiments and instructions for use in reducing the viability of a cancer cell or a tumor cell.
Embodiment 31. An isolated nucleic acid encoding an antibody of any one of embodiments 1-29.
Embodiment 32. The isolated nucleic acid of embodiment 31, the nucleic acid encoding a light chain variable domain and a heavy chain variable domain, wherein the portion of the nucleic acid encoding the light chain variable domain comprises: a CDR L1 nucleotide sequence having at least 90% sequence identity to a light chain CDR L1 nucleotide sequence selected from Table 2, a CDR L2 nucleotide sequence having at least 90% sequence identity to a light chain CDR L2 nucleotide sequence selected from Table 2, and a light chain CDR L3 nucleotide sequence having at least 90% sequence identity to a light chain CDR L3 nucleotide sequence selected from Table 2; and wherein the portion of the nucleic acid encoding the heavy chain variable domain comprises: a CDR H1 nucleotide sequence having at least 90% sequence identity to a heavy chain CDR H1 nucleotide sequence selected from Table 2, a CDR H2 nucleotide sequence having at least 90% sequence identity to a heavy chain CDR H2 nucleotide sequence selected from Table 2, and a CDR H3 nucleotide sequence having at least 90% sequence identity to a heavy chain CDR H3 nucleotide sequence selected from Table 2.
Embodiment 33. The isolated nucleic acid of embodiment 31 comprising a light chain variable domain nucleotide sequence and a heavy chain variable domain nucleotide sequence, wherein the light chain variable domain nucleotide sequence comprises: a CDR1 (CDR L1) nucleotide sequence as set forth in SEQ ID NO:137, a CDR2 (CDR L2) nucleotide sequence as set forth in SEQ ID NO:138 and a CDR3 as set forth in SEQ ID NO:139; or wherein the heavy chain variable domain nucleotide sequence comprises: a CDR1 (CDR H1) nucleotide sequence as set forth in SEQ ID NO:140, a CDR2 (CDR H2) nucleotide sequence as set forth in SEQ ID NO:141, and a CDR3 (CDR H3) nucleotide sequence as set forth in SEQ ID NO:142.
Embodiment 34. The isolated nucleic acid of embodiment 33, wherein the light chain variable domain sequence comprises a nucleic acid sequence with at least 80%, 85%, 90%, 95%, 98%, 99%, or 100% sequence identity with a light chain variable domain nucleic acid sequence selected from Table 2.
Embodiment 35. The isolated nucleic acid of embodiment 33 or embodiment 34, wherein the heavy chain variable domain comprises a nucleic acid sequence with at least 80%, 85%, 90%, 95%, 98%, 99%, or 100% sequence identity with a heavy chain variable domain nucleic acid sequence selected from Table 2.
Embodiment 36. A cell comprising the isolated nucleic acid of any one of embodiments 32-35.
Embodiment 37. A pharmaceutical composition comprising a therapeutically effective amount of an antibody of any one of embodiments 1-29 and a pharmaceutically acceptable excipient.
Embodiment 38. The pharmaceutical composition of embodiment 37, wherein the pharmaceutical composition is formulated in an aqueous solution comprising the antibody at a concentration of 0.200 mg/mL to 2.00 mg/mL, 0.500 mg/mL to 1.50 mg/mL, 0.700 mg/mL to 0.875 mg/mL, or 0.875 mg/mL to 1.25 mg/mL.
Embodiment 39. The pharmaceutical composition of embodiment 37 or embodiment 38, wherein the pharmaceutical composition is formulated for a delivery method selected from intratumoral delivery, intravenous delivery, intramuscular delivery, peritoneal delivery, or subcutaneous delivery.
Embodiment 40. The pharmaceutical composition of any one of embodiments 37-39, further comprising a therapeutically effective amount of an antineoplastic agent.
Embodiment 41. A method of treating a subject in need thereof, the method comprising administering to a subject a therapeutically effective amount of an antibody of any one of embodiments 1-29 or a pharmaceutical composition of any one of embodiments 37-40.
Embodiment 42. The method of embodiment 41, further comprising administering a therapeutically effective amount of an antineoplastic agent.
Embodiment 43. The method of embodiment 42, wherein the effective amount of an antibody and the effective amount of the antineoplastic agent are a combined synergistic amount.
Embodiment 44. The method of any one of embodiments 41-43, wherein the subject has a tumor or a cancer, is at risk of developing the tumor or the cancer, or is suspected of having the tumor or the cancer.
Embodiment 45. The method of embodiment 44, wherein the cancer is selected from acute myeloid leukemia (AML) or acute lymphoblastic leukemia (ALL).
Embodiment 46. The method of any one of embodiments 41-45, wherein the antibody is administered to the subject at a concentration of 0.200 mg/mL to 2.00 mg/mL, 0.500 mg/mL to 1.50 mg/mL, 0.700 mg/mL to 0.875 mg/mL, or 0.875 mg/mL to 1.25 mg/mL.
Embodiment 47. Use of the antibody of any one of embodiments 1-29 in the manufacture of a medicament for the treatment of a tumor or cancer.
Embodiment 48. A method of selecting a subject for treatment, the method comprising: determining a risk for a neoplastic condition to progress in the subject; administering to the subject a dose of a medicament comprising the anti-FLT3 antibody of any one of embodiments 1-27 and a drug-linker conjugate conjugated to the antibody.
Embodiment 49. The method of embodiment 48, wherein the drug-linker conjugate is selected from GGFG-monomethyl auristatin E (MMAE), GGFG-DX8951, GGFG-DXd, MC-Val-Cit-PAB-MMAE MC-Val-Cit-PAB-DX8951, or MC-Val-Cit-PAB-DXd.

What is claimed is:

1. An anti-fms-like tyrosine kinase 3 (FLT3) antibody comprising a light chain variable domain and a heavy chain variable domain,
wherein the light chain variable domain comprises:
a light chain CDR1 having at least 90% sequence identity to a light chain CDR1 (CDR L1) amino acid sequence selected from Table 2, a light chain CDR2 (CDR L2) having at least 90% sequence identity to a light chain CDR2 amino acid sequence selected from Table 2, and a light chain CDR3 (CDR L3) having at least 90% sequence identity to a light chain CDR3 amino acid sequence selected from Table 2; and
wherein the heavy chain variable domain comprises:
a heavy chain CDR1 having at least 90% sequence identity to a heavy chain CDR1 (CDR H1) amino acid sequence selected from Table 2, a heavy chain CDR2 (CDR H2) having at least 90% sequence identity to a heavy chain CDR2 amino acid sequence selected from Table 2, and a CDR3 (CDR H3) having at least 90% sequence identity to a heavy chain CDR3 amino acid sequence selected from Table 2.

2. An anti-fms-like tyrosine kinase 3 (FLT3) antibody comprising a light chain variable domain and a heavy chain variable domain,
wherein the light chain variable domain comprises:
a light chain CDR1 (CDR L1) as set forth in SEQ ID NO:9, a light chain CDR2 (CDR L2) as set forth in SEQ ID NO:10 and a light chain CDR3 (CDR L3) as set forth in SEQ ID NO:11; and
wherein the heavy chain variable domain comprises:
a heavy chain CDR1 (CDR H1) as set forth in SEQ ID NO: 12, a heavy chain (CDR H2) as set forth in SEQ ID NO: 13, and a heavy chain (CDR H3) as set forth in SEQ ID NO:14.

3. The antibody of claim 1, wherein the light chain variable domain comprises an amino acid sequence with at least 80%, 85%, 90%, 95%, 98%, 99%, or 100% sequence identity with a light chain variable domain sequence selected from Table 2.

4. The antibody of claim 3, wherein the heavy chain variable domain comprises an amino acid sequence with at least 80%, 85%, 90%, 95%, 98%, 99%, or 100% sequence identity with a heavy chain variable domain sequence selected from Table 2.

5. The antibody of claim 1, wherein the antibody is a humanized antibody.

6. The antibody of claim 1, wherein the antibody is a chimeric antibody.

7. The antibody of claim 1, wherein the antibody is an IgG.

8. The antibody of claim 7, wherein the antibody is an IgG1.

9. The antibody of claim 1, wherein the antibody is a Fab′ fragment.

10. The antibody of claim 1, wherein the antibody is a single chain antibody (scFv).

11. The antibody of claim 1, wherein the light chain variable domain and the heavy chain variable domain form part of a scFv.

12. The antibody of claim 1, wherein the antibody is capable of binding FLT3.

13. The antibody of claim 1, wherein the antibody is bound to FLT3.

14. The antibody of claim 12, wherein the FLT3 forms part of a cell.

15. The antibody of claim 14, wherein the cell is selected from a cancer cell, a myeloid cell, or a stem cell.

16. The antibody of claim 15, wherein the cancer cell is an acute myeloid leukemia cell.

17. An anti-FLT3 antibody, wherein the anti-FLT3 antibody binds the same epitope as an antibody comprising: a heavy chain variable region domain comprising a CDR1 (CDR H1) as set forth in Table 2, a CDR2 (CDR H2) as set forth in Table 2, and a CDR3 (CDR H3) as set forth in Table 2, and a light chain variable domain comprising a CDR1 (CDR L1) as set forth in Table 2, a CDR2 (CDR L2) as set forth in Table 2, and a CDR3 (CDR L3) as set forth in Table 2.

18. The anti-FLT3 antibody of claim 17, wherein the light chain variable domain comprises an amino acid sequence with at least 80%, 85%, 90%, 95%, 98%, 99%, or 100% sequence identity with a light chain variable domain amino acid sequence as set forth in Table 2.

19. The anti-FLT3 antibody of claim 18, wherein the heavy chain variable domain comprises an amino acid sequence with at least 80%, 85%, 90%, 95%, 98%, 99%, or 100% sequence identity with a light chain variable domain amino acid sequence as set forth in Table 2.

20. The anti-FLT3 antibody of claim 1, wherein the antibody is conjugated to a payload.

21. The anti-FLT3 antibody of claim 20, wherein the payload is an antineoplastic agent.

22. The anti-FLT3 antibody of claim 20, wherein the payload is selected from monomethyl auristatin E (MMAE), DXd, or DX8951.

23. The anti-FLT3 antibody of claim 1, wherein the antibody is conjugated to a linker molecule.

24. The anti-FLT3 antibody of claim 23, wherein the linker is selected from MC-Val-Cit-PAB or a tetrapeptide.

25. The anti-FLT3 antibody of claim 24, wherein the tetrapeptide is GGFG.

26. An anti-FLT3 antibody, the antibody comprising:
a heavy chain variable region according to SEQ ID NO: 194;
a light chain variable region according to SEQ ID NO: 193; and
a drug payload.

27. The anti-FLT3 antibody of claim 26, wherein the drug payload is selected from DXd or DX8951.

28. The anti-FLT3 antibody of claim 26, wherein the drug payload is directly conjugated to the anti-FLT3 antibody via a linker.

29. The anti-FLT3 antibody of claim 28, wherein the linker is GGFG.

30. A kit comprising: the anti-FLT3 antibody of claim 1 and instructions for use in reducing the viability of a cancer cell or a tumor cell.

31. An isolated nucleic acid encoding an antibody of claim 1.

32. The isolated nucleic acid of claim 31, the nucleic acid encoding a light chain variable domain and a heavy chain variable domain,
wherein the portion of the nucleic acid encoding the light chain variable domain comprises:
a CDR L1 nucleotide sequence having at least 90% sequence identity to a light chain CDR L1 nucleotide sequence selected from Table 2, a CDR L2 nucleotide sequence having at least 90% sequence identity to a light chain CDR L2 nucleotide sequence selected from Table 2, and a light chain CDR L3 nucleotide sequence having at least 90% sequence identity to a light chain CDR L3 nucleotide sequence selected from Table 2; and
wherein the portion of the nucleic acid encoding the heavy chain variable domain comprises:
a CDR H1 nucleotide sequence having at least 90% sequence identity to a heavy chain CDR H1 nucleotide sequence selected from Table 2, a CDR H2 nucleotide sequence having at least 90% sequence identity to a heavy chain CDR H2 nucleotide sequence selected from Table 2, and a CDR H3 nucleotide sequence having at least 90% sequence identity to a heavy chain CDR H3 nucleotide sequence selected from Table 2.

33. The isolated nucleic acid of claim 31 comprising a light chain variable domain nucleotide sequence and a heavy chain variable domain nucleotide sequence,
wherein the light chain variable domain nucleotide sequence comprises:
a CDR1 (CDR L1) nucleotide sequence as set forth in SEQ ID NO:137, a CDR2 (CDR L2) nucleotide sequence as set forth in SEQ ID NO:138 and a CDR3 as set forth in SEQ ID NO:139; or
wherein the heavy chain variable domain nucleotide sequence comprises:
a CDR1 (CDR H1) nucleotide sequence as set forth in SEQ ID NO: 140, a CDR2 (CDR H2) nucleotide sequence as set forth in SEQ ID NO:141, and a CDR3 (CDR H3) nucleotide sequence as set forth in SEQ ID NO:142.

34. The isolated nucleic acid of claim 33, wherein the light chain variable domain sequence comprises a nucleic acid sequence with at least 80%, 85%, 90%, 95%, 98%, 99%, or 100% sequence identity with a light chain variable domain nucleic acid sequence selected from Table 2.

35. The isolated nucleic acid of claim 34, wherein the heavy chain variable domain comprises a nucleic acid sequence with at least 80%, 85%, 90%, 95%, 98%, 99%, or 100% sequence identity with a heavy chain variable domain nucleic acid sequence selected from Table 2.

36. A cell comprising the isolated nucleic acid of claim 32.

37. A pharmaceutical composition comprising a therapeutically effective amount of an antibody of claim 1 and a pharmaceutically acceptable excipient.

38. The pharmaceutical composition of claim 37, wherein the pharmaceutical composition is formulated in an aqueous solution comprising the antibody at a concentration of 0.200 mg/mL to 2.00 mg/mL, 0.500 mg/mL to 1.50 mg/mL, 0.700 mg/mL to 0.875 mg/mL, or 0.875 mg/mL to 1.25 mg/mL.

39. The pharmaceutical composition of claim 37, wherein the pharmaceutical composition is formulated for a delivery method selected from intratumoral delivery, intravenous delivery, intramuscular delivery, peritoneal delivery, or subcutaneous delivery.

40. The pharmaceutical composition of claim 37, further comprising a therapeutically effective amount of an antineoplastic agent.

41. A method of treating a subject in need thereof, the method comprising administering to a subject a therapeutically effective amount of an antibody of claim 1.

42. The method of claim 41, further comprising administering a therapeutically effective amount of an antineoplastic agent.

43. The method of claim 42, wherein the effective amount of an antibody and the effective amount of the antineoplastic agent are a combined synergistic amount.

44. The method of claim 41, wherein the subject has a tumor or a cancer, is at risk of developing the tumor or the cancer, or is suspected of having the tumor or the cancer.

45. The method of claim 44, wherein the cancer is selected from acute myeloid leukemia (AML) or acute lymphoblastic leukemia (ALL).

46. The method of claim 41, wherein the antibody is administered to the subject at a concentration of 0.200 mg/mL to 2.00 mg/mL, 0.500 mg/mL to 1.50 mg/mL, 0.700 mg/mL to 0.875 mg/mL, or 0.875 mg/mL to 1.25 mg/mL.

47. Use of the antibody of claim 1 in the manufacture of a medicament for the treatment of a tumor or cancer.

48. A method of selecting a subject for treatment, the method comprising:
determining a risk for a neoplastic condition to progress in the subject;
administering to the subject a dose of a medicament comprising the anti-FLT3 antibody of claim 1 and a drug-linker conjugate conjugated to the antibody.

49. The method of claim 48, wherein the drug-linker conjugate is selected from GGFG-monomethyl auristatin E (MMAE), GGFG-DX8951, GGFG-DXd, MC-Val-Cit-PAB-MMAE MC-Val-Cit-PAB-DX8951, or MC-Val-Cit-PAB-DXd.
